SEQUENCE LISTING (10 sequences)

SEQ ID NO: 1 - DNA, 19 bp, Artificial Sequence
  Description of Artificial Sequence Synthetic oligonucleotide
  siRNA of CNOT3
cactcggatactgtggaat

SEQ ID NO: 2 - DNA, 19 bp, Artificial Sequence
  Description of Artificial Sequence Synthetic oligonucleotide
  siRNA of CNOT3
gcaaaatcaaggacgacgt

SEQ ID NO: 3 - DNA, 19 bp, Artificial Sequence
  Description of Artificial Sequence Synthetic oligonucleotide
  siRNA of DDX46
gagatgaagcaagggaaaa

SEQ ID NO: 4 - DNA, 19 bp, Artificial Sequence
  Description of Artificial Sequence Synthetic oligonucleotide
  siRNA of DDX49
cgtggaacagtgtcggcag

SEQ ID NO: 5 - DNA, 19 bp, Artificial Sequence
  Description of Artificial Sequence Synthetic oligonucleotide
  siRNA of HNRNPK
cgggagcttcaatcaaaat

SEQ ID NO: 6 - DNA, 19 bp, Artificial Sequence
  Description of Artificial Sequence Synthetic oligonucleotide
  siRNA of NXF1
caatttatgactctggaga

SEQ ID NO: 7 - DNA, 19 bp, Artificial Sequence
  Description of Artificial Sequence Synthetic oligonucleotide
  siRNA of SNRNP200
ggacaggctaaacaagaag

SEQ ID NO: 8 - DNA, 19 bp, Artificial Sequence
  Description of Artificial Sequence Synthetic oligonucleotide
  siRNA of ZC3H13
cacagaacaagagtagaaa

SEQ ID NO: 9 - DNA, 19 bp, Artificial Sequence
  Description of Artificial Sequence Synthetic oligonucleotide
  siRNA of KARS
gctgtttgtgatgaagaaa

SEQ ID NO: 10 - DNA, 19 bp, Artificial Sequence
  Description of Artificial Sequence Synthetic oligonucleotide
  siRNA of EIF3D
gggaaagaaagatacaact

(19) United States
(12) Patent Application Publication
(10) Pub. No.: US 2021/0057040 A1
(43) Pub. Date: Feb. 25, 2021

(54) Gene Panel for Personalized Medicine, Method for Forming Same, and Personalized Treatment Method Using Same

(71) Applicant: Samsung Life Public Welfare Foundation, Seoul (KR)

(72) Inventors: Yeon Jeong Kim, Seoul (KR); Donghyun Park, Seoul (KR); Woong-Yang Park, me ,g meeg m (KR)

(73) Assignee: Samsung Life Public Welfare Foundation, Seoul (KR)

(21) Appl. No.: 16/966,567

(22) PCT Filed: Jan. 30, 2019
(86) PCT No.: PCT/KR2019/001247

§ 371 Date: Oct. 7, 2020

(30) Foreign Application Priority Data
Feb. 1, 2018 (KR) 10-2018-0013081

(51) Int. Cl. G16B 20/20; G16B 25/10; G16B 50/00; G16H 10/60; G16H 50/20; G16H 50/30

(52) U.S. Cl. CPC G16B 20/20; G16B 25/10; G16H 50/30; G16H 10/60; G16H 50/20; G16B 50/00

(57) ABSTRACT

Provided are a gene panel for personalized medicine, a method of constructing the same, and a personalized treatment method using the same. In one aspect, the method of constructing the gene panel, based on an individual's genomic sequence mutation information, may detect gene mutations related to intractable diseases including cancer, and therefore, personalized treatments considering cancer progression or change in patients and treatment models of the diseases may be constructed.

### TECHNICAL FIELD

The present disclosure relates to a gene panel for personalized medicine, a method of constructing the same, and a personalized treatment method using the same.

### BACKGROUND ART

Genomic data analysis has been developed based on the advancement of next-generation sequencing (NGS) equipment and IT technology that deals with large-scale information, and through human whole genome sequencing, has led to the prediction of individuals' diseases and the provision of personalized disease prevention and treatment. In addition, the development of genome analysis technology has made it possible to secure a genome analysis technology platform and sufficient data needed for clinical application. Even though patients have the same cancer, the molecular and biological characteristics of cancer vary depending on each individual, and patients also exhibit different cancer progression and response to treatment. Therefore, it is important to consider individuals' personal characteristics for prescription. For this reason, the need for personalized medicine has emerged, and there are various studies related to the development of technology for characterizing molecular and biological alterations in patients.

Since differences in disease susceptibility or treatment response between individuals are attributed to differences in genetic variations, it is possible to prevent diseases or to provide personalized medicine optimized for each individual by analyzing genomic data. Targeted therapies have enabled personalized treatment of protein and gene abnormalities of an individual patient, but there are limitations in that mutations of target proteins are limited and drugs corresponding thereto must be used in combination. Accordingly, there is a need for personalized therapies that target gene mutations in individual patients.

The present inventors found cancer cell-specific mutations in essential genes and constitutive genes which are needed for cell survival, and designed siRNAs targeting the mutations to remove only cancer cells, thereby completing the present disclosure.

### DESCRIPTION OF EMBODIMENTS

### Technical Problem

An aspect provides a gene panel for personalized medicine, the gene panel including 10 or more consecutive polynucleotides including mutations of polynucleotides of genes listed in Table 3 below, or complementary polynucleotides thereof:

TABLE 3

| | | | | | |
|---|---|---|---|---|---|
| ALYREF | DHX9 | KPNB1 | POLR2I | RPA2 | SNRPG |
| ARCN1 | DNM2 | LIAS | POMGNT2 | RPL10A | SNU13 |
| ATF7 | DYNLRB1 | LRRC59 | PPP2R1A | RPL11 | SON |
| ATP6V0D1 | EEF2 | LSM3 | PPP5C | RPL14 | SRSF1 |
| BCL2L1 | EFTUD2 | LSM4 | PRPF18 | RPL27 | SRSF3 |
| BRIX1 | EIF2B4 | LSM5 | PRPF19 | RPL30 | SUPT5H |
| C12orf66 | EIF2S2 | LSM5 | PRPF3 | RPL31 | SUPT6H |
| C14orf2 | EIF3A | MAPK6 | PRPF31 | RPL32 | SUPV3L1 |
| C16orf72 | EIF3B | MAPKAP1 | PRPF8 | RPL34 | TAF10 |
| CARS2 | EIF3D | MEN1 | PRUNE | RPL35 | TFCP2 |
| CCNL1 | EIF3E | MRPS31 | PSMA1 | RPL35A | TFIP11 |
| CCT3 | EIF3G | MSANTD3 | PSMA2 | RPL4 | THOP1 |
| CCT4 | EIF3I | NACA | PSMA3 | RPL6 | TIMM10 |
| CCT6A | EIF4A3 | NAPA | PSMA6 | RPK2 | TIMM50 |
| CCT7 | EIF5B | NDUFV2 | PSMB2 | RPS13 | TMEM5 |
| CCT8 | EIF6 | NEDD8 | PSMB3 | RPS24 | TSG101 |
| CDC40 | ELP4 | NUDT21 | PSMB4 | RPS5 | TSTA3 |
| CDC5L | ERH | NUP133 | PSMB7 | RPS6 | TUBA1B |
| CHD4 | ETF1 | NUP54 | PSMC2 | RRM1 | TUBA1C |
| CHMP2A | EXOSC10 | NXF1 | PSMC3 | RUVBL1 | TUBB |
| CLTC | FIP1L1 | OAZ2 | PSMC4 | SDAD1 | TUBGCP2 |
| CNOT3 | FLAD1 | PABPN1 | PSMD1 | SEC61A1 | U2AF1 |
| COPA | FTSJ3 | PAPOLA | PSMD11 | SF3A1 | U2AF2 |
| COPB1 | FUBP1 | PCBP2 | PSMD12 | SF3B1 | UBA1 |
| COPB2 | GNL3 | PELP1 | PSMD3 | SF3B2 | UBB |
| COPS2 | HAUS7 | PFDN2 | PSMD6 | SF3B3 | USP39 |
| COPS4 | HDAC3 | PHB | PSMD7 | SF3B4 | VCP |
| COPS6 | HNRNPC | PHB2 | PSMD8 | SF3B5 | VPS28 |
| COPS8 | HNRNPK | PHF5A | PUF60 | SKIV2L2 | WBP11 |
| COFZ1 | HNRNPL | PI4KA | QARS | SNAPC3 | WDR12 |
| DARS | HNRNPM | PLIN3 | RALY | SNRNP200 | WDR61 |
| DDB1 | HNRNPU | PLRG1 | RAN | SNRNP27 | WWP2 |
| DDX18 | HSPA9 | PNPT1 | RANBP2 | SNRNP70 | XPO1 |
| DDX41 | IARS2 | POLR2A | RBM14 | SNRPC | YY1 |
| DDX46 | ICK | POLR2D | RBM17 | SNRPD1 | ZC3H13 |
| DDX49 | KARS | POLR2F | RBM39 | SNRPD2 | ZNF207 |
| ZNF22 | ZNF24 | | | | |

Another aspect provides a method of constructing a gene panel for personalized medicine, the method including constructing a target gene set by screening the intersection of essential genes and constitutive genes.

Still another aspect provides a method of providing information for personalized treatment, the method including detecting mutations of the gene panel defined as above in a biological sample isolated from an individual; and from the detection results, determining, as a treatment target for the individual, genes in which mutations have occurred.

### Solution to Problem

An aspect provides a gene panel for personalized medicine, the gene panel including 10 or more consecutive polynucleotides including mutations of polynucleotides of genes listed in Table 3 below, or complementary polynucleotides thereof:

TABLE 3

| | | | | | |
|---|---|---|---|---|---|
| ALYREF | DHX9 | KPNBI | POLR2I | RPA2 | SNRPG |
| ARCN1 | DNM2 | LIAS | POMGNTS | RPL10A | SNU13 |
| ATF7 | DYNLRB1 | LRRC59 | PPP2R1A | RPL11 | SON |
| ATP6V0D1 | EEF2 | LSM3 | PPP5C | RPL14 | SRSF1 |
| BCL2L1 | EFTUD2 | LSM1 | PRPF18 | RPL27 | SRSF3 |
| BRIX1 | EIF2B4 | LSM5 | PRPF19 | RPL30 | SUPT5H |
| C12orf66 | EIF2S2 | LSM6 | PRPF3 | RPL31 | SUPT6H |
| C14orf2 | EIF3A | MAPK6 | PRPF31 | RPL32 | SUPV3L1 |
| C16orf72 | EIF3B | MAPKAP1 | PRPF8 | RPL34 | TAF10 |
| CARS2 | EIF3D | MEN1 | PRUNE | RPL35 | TFCP2 |
| CCNL1 | EIF3E | MRPS31 | PSMA1 | RPL35A | TFIPI1 |
| CCT3 | EIF3G | MSANTD3 | PSMA2 | RPL4 | THOP1 |
| CCT4 | EIF3I | NACA | PSMA3 | RPL6 | TIMM10 |
| CCT6A | EIF4A3 | NAPA | PSMA6 | RPN2 | TIMM50 |
| CCT7 | EIF5B | NDUFV2 | PSMB2 | RPS13 | TMEM5 |
| CCT8 | EIF6 | NEDD8 | PSMB3 | RPS24 | TSG101 |
| CDC40 | ELP4 | NUDT21 | PSMB4 | RPS5 | TSTA3 |
| CDC5L | ERH | NUP133 | PSMB7 | RPS6 | TUBA1B |
| CHD4 | ETF1 | NUP54 | PSMC2 | RRM1 | TUBA1C |
| CHMP2A | EX0SC10 | NXF1 | PSMC3 | RUVBL1 | TUBB |
| CLTC | FIP1L1 | OAZ2 | PSMC4 | SDAD1 | TUBGCP2 |
| CNOT3 | FLAD1 | PABPN1 | PSMD1 | SEC61A1 | U2AF1 |
| COPA | FTSJ3 | PAPOLA | PSMD11 | SF3A1 | U2AF2 |
| COPB1 | FUBP1 | PCBP2 | PSMD12 | SF3B1 | UBA1 |
| COPB2 | GNL3 | PELP1 | PSMD3 | SF3B2 | UBB |
| COPS2 | HAUS7 | PFDN2 | PSMD6 | SF3B3 | USP39 |
| COPS4 | HDAC3 | PHB | PSMD7 | SF3B4 | VCP |
| COPS6 | HNRNPC | PHB2 | PSMD8 | SF3B5 | VPS28 |
| COPS8 | HNRNPK | PHP5A | PUF60 | SKIV2L2 | WBP11 |
| COPZ1 | HNRNPL | PI4KA | QARS | SNAPC3 | WDR12 |
| DARS | HNRNPM | PL1N3 | RALY | SNRNP200 | WDR61 |
| DDB1 | HNRNPU | PLRC1 | RAN | SNRNP27 | WWP2 |
| DDX18 | HSPA9 | PNPT1 | RANBP2 | SNRNP70 | XPO1 |
| DDX41 | IARS2 | POLR2A | RBM14 | SNRPC | YY1 |
| DDX46 | ICK | POLR2D | RBM17 | SNRPD1 | ZC3H13 |
| DDX49 | KARS | POLR2F | RBM39 | SNRPD2 | ZNF207 |
| ZNF22 | ZNF24 | | | | |

The “polynucleotide” may be DNA or RNA. Further, the polynucleotide may be single-stranded or double-stranded. Further, the polynucleotide may include those consisting of natural nucleotides as well as a nucleotide selected from the group consisting of natural nucleotides, analogues of natural nucleotides, nucleotides having modifications in a sugar base, or phosphate moiety of natural nucleotides, and combinations thereof, as long as it is able to hybridize with a complementary nucleotide by hydrogen bonds (Scheit, Nucleotide Analogs, John Wiley, New York (1980); Uhlman and Peyman, Chemical Reviews, 90:543-584 (1990)).

The polynucleotide shows a single nucleotide polymorphism on a mutation site. Therefore, when one single-stranded polynucleotide is associated with the risk of developing intractable diseases including cancer, it may be determined that a polynucleotide complementary to the single-stranded polynucleotide is naturally associated with the risk of developing intractable diseases including cancer. For example, in a polynucleotide of SEQ ID NO: 1, a nucleotide at position of hg19. 54656673 is “C or T”. In this case, the gene panel includes the “C or T” nucleotide at position of hg19. 54656673, and 10 or more consecutive nucleotides selected from the polynucleotide of SEQ ID NO: 1, as well as a complementary single-stranded polynucleotide having a “C or T” nucleotide at position corresponding to the position of hg19. 54656673.

The polynucleotides may be primers, probes, or antisense nucleic acids. The “primer” refers to a single-stranded polynucleotide that may act as a starting point in nucleotide polymerization by a polymerase. For example, the primer may be a single-stranded polynucleotide capable of acting as a starting point of template-directed DNA synthesis under appropriate conditions of an appropriate temperature and an appropriate buffer (i.e., in the presence of four different nucleoside triphosphates and a polymerase). The appropriate length of the primer may vary depending on various factors, for example, temperature and use of the primer. The primer may have a length of 15 nt to 30 nt. Short primer molecules generally require lower temperatures to form a sufficiently stable hybrid complex with a template.

The sequence of the primer does not need to be completely complementary to some sequences of the template, but the primer should have sufficient complementarity within a range where it may perform its inherent actions through hybridization with the template. Accordingly, the primer includes not only the polynucleotide itself, but also a sequence that specifically hybridizes with the polynucleotide, which may serve as a starting point for polymerization. For example, the primer may be a sequence perfectly complementary to the polynucleotides of SEQ ID NOS: 1 to 10 as well as a sequence having complementarity within a range where it may act as a primer through hybridization with the sequences. The design of the primer may be easily carried out by a person skilled in the art by referring to a sequence of a given target nucleotide sequence to be amplified. For example, the primer may be designed using commercially available primer design programs. Examples of the commercially available primer design programs may include program PRIMER 3.

When the polynucleotide is used as a PCR primer, in addition to the polynucleotide, a primer specifically binding to a complementary strand thereof may be included.

The “probe” refers to a polynucleotide that specifically binds to a specific target sequence. The polynucleotide may be DNA or RNA. The polynucleotide may be single-stranded. Further, the polynucleotide may include those consisting of natural nucleotides as well as a nucleotide selected from the group consisting of natural nucleotides, analogues of natural nucleotides, nucleotides having modifications in a sugar base, or phosphate moiety of natural nucleotides, and combinations thereof, as long as it is able to hybridize with a complementary nucleotide by hydrogen bonds. The probe may have a length of 5 nt to 100 nt, 10 nt to 90 nt, 15 nt to 80 nt, 20 nt to 70 nt, or 30 nt to 50 nt. The polynucleotide includes PNA. In addition, the polynucleotide may have a detectable label (e.g., Cy3, Cy5 fluorescent substance) attached, for example, to the 3′-end or 5′-end, in order to facilitate detection of the polynucleotide or a complex, to which the polynucleotide is bound, in the analysis reaction.

The probe may be a nucleotide sequence perfectly complementary to a target sequence including a mutation site. In addition, the probe may have a substantially complementary nucleotide sequence within a range that does not interfere with specific hybridization to the target sequence including the mutation site. Further, the probe may have a modified nucleotide within a range that does not impair specific hybridization to the target sequence including the mutation site. Examples of the probe may be selected from the group consisting of a perfect match probe consisting of a sequence perfectly complementary to the polynucleotide including the mutation site and a probe having a sequence perfectly complementary to all sequence excluding the mutation site with regard to the polynucleotide including the mutation site.

The “antisense nucleic acid” refers to a nucleic acid-based molecule that has a complementary nucleotide sequence with respect to a target sequence and is able to form a dimer therewith. The antisense nucleic acid may be the polynucleotide or a fragment thereof, or those complementary thereto. The antisense nucleic acid may have a length of 10 nt or more, more specifically, 10 nt to 200 nt, 10 nt to 150 nt, or 10 nt to 100 nt, but an appropriate length may be selected to increase detection specificity.

By using the above primer, probe, or antisense nucleic acid, a nucleotide sequence having a specific allele at the mutation site may be amplified or the presence thereof may be identified.

The polynucleotides may be polynucleotides labeled with a detectable label. The detectable label, which is a labeling material capable of generating a detectable signal, may be a labeling material capable of generating a detectable signal, including a fluorescent material, for example, a material such as Cy3 and Cy5. The detectable label may identify results of nucleic acid hybridization.

In addition, the mutations may be silent mutations. Specifically, amino acid substitution means that an amino acid sequence is changed by change of one or more nucleotides. The silent mutation means that even though nucleotides are changed, an amino acid encoded thereby is not changed. The mutations of the polynucleotides may include T at position of hg19. 54656673 in SEQ ID NO: 1, A at position of hg19. 54649456 in SEQ ID NO: 2, G at position of hg19. 134109517 in SEQ ID NO: 3, G at position of hg19. 19030598 in SEQ ID NO: 4, A at position of hg19. 86585178 in SEQ ID NO: 5, T at position of hg19. 62564024 in SEQ ID NO: 6, A at position of hg19. 96950285 in SEQ ID NO: 7, A at position of hg19. 46543189 in SEQ ID NO: 8, C at position of hg19. 75663394 in SEQ ID NO: 9, and T at position of hg19. 36912832 in SEQ ID NO: 10. Further, the gene panel according to one embodiment may be for targeted treatment of a cancer or an intractable disease, and the cancer may be, for example, brain cancer, gastric cancer, lung cancer, breast cancer, ovarian cancer, liver cancer, bronchial cancer, nasopharyngeal cancer, laryngeal cancer, esophageal cancer, pancreatic cancer, bladder cancer, prostate cancer, colorectal cancer, colon cancer, bone cancer, skin cancer, thyroid cancer, parathyroid cancer, ureter cancer, or cervical cancer. The intractable disease may be, for example, albinism, alcaptonuria, lactose intolerance, hereditary hemorrhagic telangiectasia, thalassemia, congenital dyserythropoietic anaemia, Evans syndrome, pituitary hypofunction, Huntington's disease, hereditary motor sensory neuropathy, or other autonomic nervous system disorders.

As described above, the gene panel according to one specific embodiment is based on an individual patient's tumor genes, and thus tumor treatment effects may be improved through a personalized gene therapy. In addition, since the gene panel targets not only the existing missense mutations, but also silent mutations in which protein mutations do not occur, it is possible to effectively perform a targeted therapy for various diseases caused by gene mutations.

Another aspect provides a method of constructing a gene panel for personalized medicine, the method including constructing a target gene set by screening the intersection of essential genes and constitutive genes. Specifically, the essential genes, which are genes essential for cell survival, encode proteins for maintaining functions such as cell metabolism, DNA replication, protein translation, etc. The essential genes may be those shown in Table 1 below. In addition, the constitutive genes, which are genes originally possessed by living organisms and always fulfill their functions regardless of environmental conditions, may be selected from housekeeping genes which are always expressed in cells and indispensable for cell survival. The constitutive genes may be those shown in Table 2 below. Further, the genes forming the intersection of the essential genes and the constitutive genes are essential for cell survival, and may transcribe genes regardless of cellular environmental conditions. The genes may be those shown in Table 3 below.

Further, the method may include extracting mutant genes common to the target gene set from a plurality of public cell line databases, and collecting base sequence information of the mutant genes; and verifying each mutant gene from the base sequence information of the mutant genes. The method of constructing the gene panel for personalized medicine according to one specific embodiment includes extracting mutant genes common to the target gene set from a plurality of public cell line databases, and collecting base sequence information of the mutant genes. Specifically, the plurality of public cell line databases may include cancer databases of Cancer Cell Line Encyclopedia (CCLE), National Cancer Institute (NCI), or Catalogue of Somatic Mutations in Cancer (COSMIC). The extracting of the mutant genes refers to identifying and/or discriminating information about nucleotide substitution, addition, or deletion in a nucleotide sequence that constitutes an exon of a subject gene. Such nucleotide substitution, addition, or deletion may occur due to various causes, and may be caused by structural differences including, for example, mutations, cleavage, deletions, duplications, inversions and/or translocations of chromosomes. The extracting of the mutant gene may be performed by mapping the genome sequencing data to a standard base sequence. In addition, the collecting of the base sequence information of the mutant genes may be performed by obtaining genome sequencing data of a subject. In this regard, the genome sequencing data may be exome sequencing data, whole genome sequencing data, or sequence data of a gene known to be related to a disease.

Further, the method of constructing the gene panel for personalized medicine according to one specific embodiment includes verifying each mutant gene from the base sequence information of the mutant genes. Specifically, the verifying of the mutant gene may be performed by comparing expression levels between a normal gene and the gene in which the mutation has occurred.

Still another aspect provides a method of providing information for personalized treatment, the method including detecting mutations of the gene panel defined as above in a biological sample isolated from an individual; and from the detection results, determining, as a treatment target for the individual, genes in which mutations have occurred.

The method of providing information according to one specific embodiment includes detecting mutations of the gene panel defined as above in a biological sample isolated from an individual. Specifically, the individual refers to a subject for predicting the risk of developing a cancer or an intractable disease caused by gene mutations. The individual may include vertebrates, mammals, or humans (Homo sapiens). For example, the human may be Korean. Further, the biological sample may be a tissue, a cell, whole blood, serum, plasma, saliva, sputum, a cerebrospinal fluid, or urine. The detecting of mutations of the gene panel defined as above may be performed by isolating a nucleic acid from the biological sample and then determining the mutation site. The methods of isolating the nucleic acid and determining the mutation site are known in the art. The method of isolating the nucleic acid may be performed by, for example, directly isolating DNA from the biological sample or amplifying a specific region by a nucleic acid amplification method such as PCR. The isolated nucleic acid sample includes not only purely isolated nucleic acids, but also crudely isolated nucleic acids, for example, cell lysates containing nucleic acids. The nucleic acid amplification method includes PCR, ligase chain reaction (LCR), transcription amplification, self-sustained sequence replication, and nucleic acid-based sequence amplification (NASBA). The isolated nucleic acid may be DNA or RNA. The DNA may be genomic DNA, cDNA, or recombinant DNA. The RNA may be mRNA. Further, the method of determining the mutation site may directly determine nucleotides at the mutation site by, for example, a known nucleotide sequencing method. The method of determining nucleotide sequences may include the Sanger (or dideoxy) sequencing method or the Maxam-Gilbert (chemical cleavage) method. In addition, the nucleotide at the mutation site may be determined by hybridizing a probe including the sequence of the mutation site with a target polynucleotide and analyzing the hybridization result. The degree of hybridization may be confirmed by, for example, labeling a detectable label on a target nucleic acid and detecting the hybridized target nucleic acid, or may be confirmed by an electrical method. A single base primer extension (SBE) method may also be used.

The method of providing information of one specific embodiment includes determining a gene, in which mutations have occurred, as a treatment target of the individual, based on the detection result. Specifically, when the mutations of the gene panel include Tat position of hg19. 54656673 in SEQ ID NO: 1, A at position of hg19. 54649456 in SEQ ID NO: 2, G at position of hg19. 134109517 in SEQ ID NO: 3, G at position of hg19. 19030598 in SEQ ID NO: 4, A at position of hg19. 86585178 in SEQ ID NO: 5, T at position of hg19. 62564024 in SEQ ID NO: 6, A at position of hg19. 96950285 in SEQ ID NO: 7, A at position of hg19. 46543189 in SEQ ID NO: 8, C at position of hg19. 75663394 in SEQ ID NO: 9, and T at position of hg19. 36912832 in SEQ ID NO: 10, it is determined that the individual belongs to a group with a high risk of developing cancer caused by gene mutations. In addition, by identifying the gene in which the mutation has occurred, it is possible not only to predict the risk of developing a specific disease, but also to determine the gene as a treatment target for a specific disease of an individual, thereby performing an efficient targeted therapy.

### Advantageous Effects of Disclosure

A gene panel of one aspect, based on an individual's genomic sequence mutation information, may detect gene mutations related to intractable diseases including cancer, and therefore, personalized treatments considering cancer progression or change in patients and treatment models of the diseases may be constructed. In addition, by broadening understanding of the molecular mechanisms of tumor evolution and carcinogenesis through identification of an individual's genome mutations, it is possible to efficiently discover new drugs personalized for patients, and it may also be a starting point for the development of a personalized therapy.

### BRIEF DESCRIPTION OF DRAWINGS

FIG. 1 shows a schematic illustration of a personalized treatment process using genes essential for cell survival;

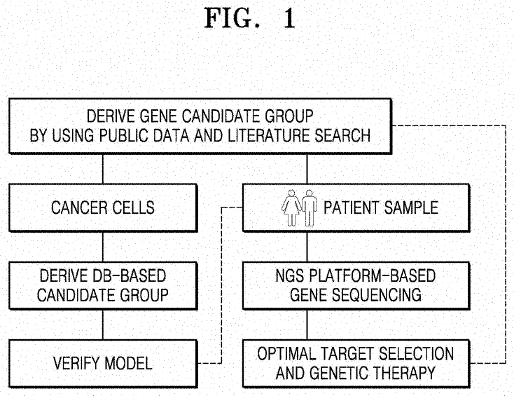

FIG. 2 shows a schematic illustration of a process of deriving essential genes and constitutive genes from housekeeping genes;

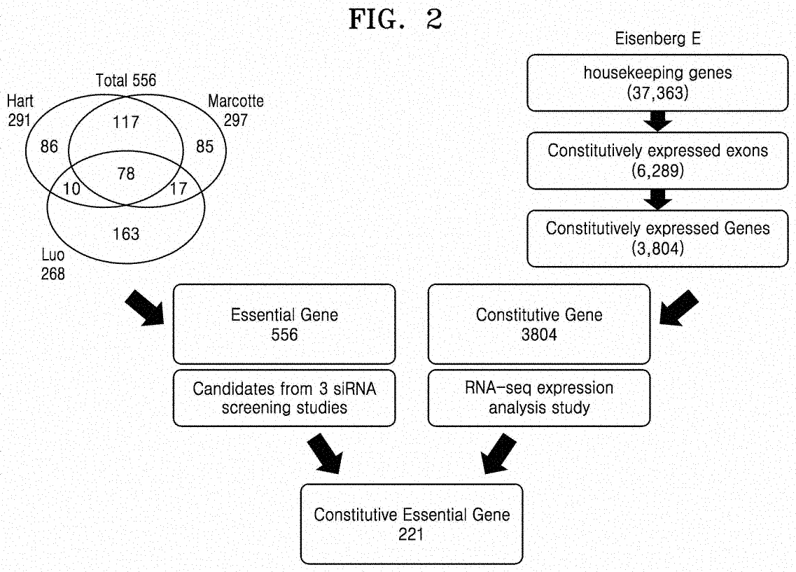

FIG. 3 shows results of analyzing WES/WGS public data of cancer cell lines;

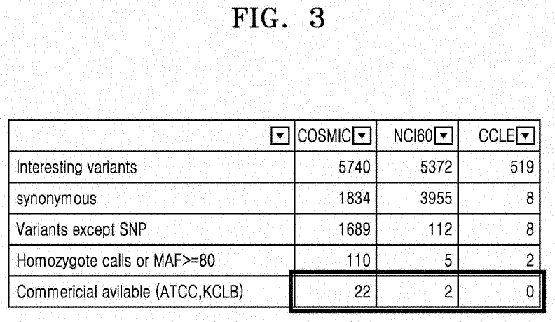

FIG. 4 shows results of examining inhibition of protein expression of mutant genes by siRNAs designed in Example 1-3; and

FIG. 5 shows results of examining cytotoxicity of siRNAs designed in Example 1-3 against cancer cells.

### MODE OF DISCLOSURE

Hereinafter, exemplary embodiments are provided for better understanding of the present disclosure. However, the following exemplary embodiments are provided for easier understanding of the present disclosure, and the contents of the present disclosure are not limited by the following exemplary embodiments.

### Example

### Example 1. Construction and Verification of Gene Panel

1-1. Selection of Constitutive Genes

Through literature search, 556 essential genes were selected based on siRNA screening, and 3804 genes (constitutive genes) present in all cells above a predetermined level were selected from housekeeping genes through RNA Seq expression. The related literatures are as follows.

Measuring error rates in genomic perturbation screens: gold standards for human functional genomics (Mol Syst Biol. 2014 July; 10(7): 733.)

Highly parallel identification of essential genes in cancer cells (Proc Natl Acad Sci USA. 2008 Dec. 23; 105(51): 20380-20385.)

Essential Gene Profiles in Breast, Pancreatic, and Ovarian Cancer Cells (Cancer Discov. 2012 February; 2(2):172-189. doi: 10.1158/2159-8290.)

Thereafter, the intersection (220 genes) of the essential genes and the housekeeping genes were selected as screening gene sets. The essential genes, constitutive genes, and gene sets are shown in Tables 1 to 3 below.

TABLE 1

ADCYAP1R1
ADRBK2
AKAP8L
ALF
ALG10B
ALYREF
ANAPC4
ANXA1
APOBEC3F
AQR
ARCN1
ARF4
ARHGEF4
ARS2
ASCC3L1
ATF7
ATP5I
ATP6V0D1
AURKB
AYTL1
B3GALNT2
BCL2L1
BDKRB1
BMPR1A
BOP1
BRIX1
BST2
BUB1B
C12orf66
C14ORF166
C14orf2
C16orf72
C1ORF141
C22orf27
C3orf39
C9orf30
CACNA1B
CAPN2
CARS2
CCNB2
CCNB3
CCNL1
CCT3
CCT4
CCT6A
CCT7
CCT8
CD70
CDC2L1
CDC40
CDC5L
CDK11B
CDK17
CEND1
CENPO
CENTD2
CHD4
CHEK1
CHMP2A
CHRM1
CLEC2D
CLTC
CMTM3
CNOT3
COPA
COPB1
COPB2
COPS2
COPS4
COPS6
COPS8
COPZ1
COPZ2
CPA4
CPSF1
CREBL2
CSE1L
CTDNEP1
CYorf15A
CYP1A2
DAPK1
DARS
DBF4
DCTN1
DDB1
DDR1
DDR2
DDX18
DDX41
DDX46
DDX49
DDX51
DHCR7
DHX9
DLC1
DLGAP1
DMKN
DMPK
DNAJB6
DNM2
DUOXA2
DUSP7
DYNC1H1
DYNC1I2
DYNLRB1
ECT2
EEF2
EFTUD2
EIF1AX
EIF2B4
EIF2S2
EIF3A
EIF3B
EIF3C
EIF3D
EIF3F
EIF3G
EIF3I
EIF3S4
EIF3S6
EIF4A3
EIF4E3
EIF5B
ELP4
EPHB4
EPS8
ERBB3
ERCC6L
ERH
ESPL1
ETF1
EVX2
EXOSC10
FAM126A
FAM164C
FIP1L1
FLAD1
FLJ16008
FLJ20105
FLT3LG
FNDC7
FOS
FOXM1
FTSJ3
FUBP1
FUT9
GAR1
GGPS1
GIMAP4
GLIPR1L2
GLT25D2
GNA14
GNG11
GNL3
GPC6
GPD2
GPR112
GREM1
GRIN1
GRIN2C
GRPEL2
GTDC2
GTF2E2
GTF3C4
GUSBP4
HAUS7
HDAC3
HEATR1
HINT3
HLA-DRB3
HNRNPC
HNRNPK
HNRNPM
HNRNPU
HNRPC
HNRPL
HNRPM
HNRPU
HOXA2
HOXC13
HSDL2
HSPA9
HSPE1
HSPG2
IARS2
ICK
IGFBP2
IL28A
INSR
INTS4L1
INTS4L2
INTS9
IRS4
ISG20
ITGA2
ITGB4BP
KARS
KCNC3
KCNJ5
KCTD15
KDELR3
KIAA0226
KLHL14
KPNB1
KRAS
KSR1
LCN10
LDB1
LDHC
LIAS
LILRA2
LOC283816
LOC392254
LOC401361
LOC402530
LOC402677
LRRC59
LSM3
LSM4
LSM5
LSM6
LY96
M6PRBP1
MAOA
MAPK6
MAPKAP1
MBL2
MCM2
MDN1
MED30
MEN1
MPL
MRPS31
MS4A13
MSANTD3
MYB
MYO3B
NACA
NACAP1
NAPA
NAPG
NCBP1
NCOR2
NDUFA4L2
NDUFAB1
NDUFV2
NEDD8
NHP2L1
NID2
NLN
NOC4L
NR1D1
NR2F1
NR3C1
NUDT21
NUDT4
NUF2
NUP133
NUP205
NUP54
NUP88
NUP93
NUP98
NUS1
NXF1
OAZ2
OR2J2
OR51B4
OR51E1
OR51E2
ORM2
PABPN1
PAPOLA
PAQR5
PAX5
PCBP2
PDE1B
PDPK1
PELP1
PFDN2
PGLYRP4
PHB
PHB2
PHF5A
PIK4CA
PISD
PJA2
PLD1
PLK1
PLK1S1
PLRG1
PNPT1
POLA1
POLR2A
POLR2D
POLR2F
POLR2H
POLR2T
PPP2R1A
PPP5C
PRDM16
PRIM2A
PRKAB2
PRPF18
PRPF19
PRPF3
PRPF31
PRPF38A
PRPF8
PRTN3
PRUNE
PSMA1
PSMA2
PSMA3
PSMA6
PSMB2
PSMB3
PSMB4
PSMB7
PSMC1
PSMC2
PSMC3
PSMC4
PSMD1
PSMD11
PSMD12
PSMD3
PSMD6
PSMD7
PSMD8
PTCH2
PTGDR2
PTPN9
PTPRC
QARS
RAD51
RALY
RAN
RANBP2
RBM14
RBM17
RBM39
RBM47
RBMXL1
RFC4
RFTN1
RGS13
RGS17
RGS18
RILPL2
RPA1
RPA2
RPL10
RPL10A
RPL11
RPL12
RPL13
RPL13A
RPL13AP6
RPL14
RPL17
RPL18
RPL18A
RPL19
RPL23
RPL23A
RPL24
RPL26
RPL27
RPL3
RPL30
RPL31
RPL32
RPL34
RPL35
RPL35A
RPL36
RPL37
RPL37A
RPL38
RPL39
RPL4
RPL5
RPL6
RPL7
RPL7A
RPL9
RPLP0
RPLP1
RPLP2
RPN2
RPRD1A
RPS11
RPS12
RPS13
RPS14
RPS15
RPS15A
RPS17
RPS18
RPS19
RPS20
RPS24
RPS25
RPS26
RPS27
RPS27A
RPS28
RPS29
RPS3
RPS3A
RPS4X
RPS5
RPS6
RPS7
RPS8
RPS9
RPSA
RPSAP58
RPSAP9
RPTOR
RRM1
RTF1
RUVBL1
RUVBL2
RXRG
RY1
SCRG1
SDAD1
SEC14L3
SEC61A1
SF3A1
SF3A2
SF3B1
SF3B2
SF3B3
SF3B4
SF3B5
SFPQ
SFRS1
SHFM1
SIAHBP1
SIP1
SKAP1
SKIV2L2
SLC22A3
SLC26A6
SLC2A2
SMARCC1
SMARCC2
SMC2
SMC3
SMG1
SNAPC3
SNRNP200
SNRNP27
SNRNP70
SNRPC
SNRPD1
SNRPD2
SNRPE
SNRPG
SON
SP140
SPINT2
SPRED1
SRCAP
SRFBP1
SRRM2
SRRT
SRSF1
SRSF3
ST18
STAG2
STK3
STRN
SUPT5H
SUPT6H
SUPV3L1
SV2A
TAF10
TAF2
TAF4B
TBC1D25
TEKT2
TEX11
TFCP2
TFIP11
TGIF2LX
THOC4
THOP1
THRAP6
TIE1
TIGD2
TIMM10
TIMM50
TIPARP
TLE2
TLR9
TLX3
TM7SF2
TMEM5
TMOD3
TNFRSF1A
TPR
TRIM13
TRIP13
TRPV6
TSG101
TSTA3
TTC23
TUBA1B
TUBA1C
TUBB
TUBGCP2
TXK
TXLNG2P
TXNDC5
U2AF1
U2AF2
UBA1
UBB
UCP2
UNC13B
USP17L4
USP39
VCP
VLDLR
VPS28
VWCE
WBP11
WDR12
WDR60
WDR61
WDR86
WWP2
XAB2
XBP1
XCL1
XCL2
XPO1
YY1
ZAN
ZBTB48
ZC3H13
ZFP37
ZFX
ZNF141
ZNF155
ZNF160
ZNF207
ZNF22
ZNF24
ZNF266
ZNF471
ZNRF2

TABLE 2

| [symbol] | [symbol] |
|---|---|
| AAAS | NM_015665 |
| AAGAB | NM_024666 |
| AAMP | NM_001087 |
| AAR2 | NM_015511 |
| AARS | NM_001605 |
| AARS2 | NM_020745 |
| AARSD1 | NM_001261434 |
| AASDHPPT | NM_015423 |
| AATF | NM_012138 |
| ABCB10 | NM_012089 |
| ABCB7 | NM_004299 |
| ABCD3 | NM_002858 |
| ABCE1 | NM_002940 |
| ABCF1 | NM_001090 |
| ABCF2 | NM_005692 |
| ABCF3 | NM_018358 |
| ABHD10 | NM_018394 |
| ABHD12 | NM_015600 |
| ABHD13 | NM_032859 |
| ABHD14A | NM_015407 |
| ABHD16A | NM_021160 |
| ABHD4 | NM_022060 |
| ABHD8 | NM_024527 |
| ABI1 | NM_005470 |
| ABT1 | NM_013375 |
| ACAD9 | NM_014040 |
| ACADVL | NM_000018 |
| ACAP3 | NM_030649 |
| ACBD3 | NM_022735 |
| ACBD5 | NM_145698 |
| ACBD6 | NM_032360 |
| ACIN1 | NM_014977 |
| ACLY | NM_001096 |
| ACOT13 | NM_018473 |
| ACOT8 | NM_005469 |
| ACOT9 | NM_001033583 |
| ACOX1 | NM_004035 |
| ACOX3 | NM_003501 |
| ACP1 | NM_004300 |
| ACSF3 | NM_174917 |
| ACSL3 | NM_004457 |
| ACSS2 | NM_001242393 |
| ACTR10 | NM_018477 |
| ACTR1A | NM_005736 |
| ACTR1B | NM_005735 |
| ACTR5 | NM_024855 |
| ACTR8 | NM_022899 |
| ACVR1 | NM_001105 |
| ACVR1B | NM_004302 |
| ADCK2 | NM_052853 |
| ADCK4 | NM_024876 |
| ADH5 | NM_000671 |
| ADI1 | NM_018269 |
| ADIPOR1 | NM_015999 |
| ADIPOR2 | NM_024551 |
| ADK | NM_001123 |
| ADNP | NM_015339 |
| ADO | NM_032804 |
| ADPRH | NM_001125 |
| ADPRHL2 | NM_017825 |
| ADPRM | NM_020233 |
| ADSL | NM_000026 |
| AES | NM_001130 |
| AFF4 | NM_014423 |
| AFTPH | NM_017657 |
| AGFG1 | NM_004504 |
| AGGP1 | NM_018046 |
| AGPAT1 | NM_006411 |
| AGPAT3 | NM_020132 |
| AGPAT6 | NM_178819 |
| AGPS | NM_003659 |
| AHCY | NM_000687 |
| AHSA1 | NM_012111 |
| AIMP1 | NM_004757 |
| AIP | NM_003977 |
| AK2 | NM_001625 |
| AK3 | NM_016282 |
| AKAP8 | NM_005858 |
| AKAP9 | NM_005751 |
| AKIP1 | NM_020642 |
| AKIRIN1 | NM_024595 |
| AKIRIN2 | NM_018064 |
| AKR1A1 | NM_006066 |
| AKR7A2 | NM_003689 |
| AKT1 | NM_005163 |
| AKT1S1 | NM_032375 |
| AKTIP | NM_022476 |
| ALAD | NM_000031 |
| ALDH3A2 | NM_000382 |
| ALDH9A1 | NM_000696 |
| ALG11 | NM_001004127 |
| ALG5 | NM_013338 |
| ALG8 | NM_024079 |
| ALG9 | NM_024740 |
| ALKBH1 | NM_006020 |
| ALKBH2 | NM_001001655 |
| ALKBH3 | NM_139178 |
| ALKBH5 | NM_017758 |
| ALS2 | NM_020919 |
| ALYREF | NM_005782 |
| AMBRA1 | NM_017749 |
| AMD1 | NM_001634 |
| ANAPC10 | NM_014885 |
| ANAPC11 | NM_016476 |
| ANAPC13 | NM_015391 |
| ANAPC15 | NM_014042 |
| ANAPC16 | NM_173473 |
| ANAPC2 | NM_013366 |
| ANAPC5 | NM_016237 |
| ANAPC7 | NM_016238 |
| ANKFY1 | NM_016376 |
| ANKH | NM_054027 |
| ANKHD1 | NM_017747 |
| ANKHD1-E | NM_020690 |
| IF4EBP3 | |
| ANKRD10 | NM_017664 |
| ANKRD17 | NM_032217 |
| ANKRD28 | NM_001195098 |
| ANKRD39 | NM_016465 |
| ANKRD46 | NM_198401 |
| ANO6 | NM_001025356 |
| ANP32A | NM_006305 |
| ANP32B | NM_006401 |
| ANP32C | NM_012403 |
| ANP32E | NM_030920 |
| ANXA6 | NM_001155 |
| ANXA7 | NM_001156 |
| AP1B1 | NM_001127 |
| AP1G1 | NM_001128 |
| AP1M1 | NM_032493 |
| AP2A1 | NM_014203 |
| AP2A2 | NM_012305 |
| AP2M1 | NM_004068 |
| AP2S1 | NM_004069 |
| AP3B1 | NM_003664 |
| AP3D1 | NM_003938 |
| AP3M1 | NM_012095 |
| AP3S1 | NM_001284 |
| AP3S2 | NM_005829 |
| AP4B1 | NM_006594 |
| AP5M1 | NM_018229 |
| APEH | NM_001640 |
| APEX1 | NM_001641 |
| APEX2 | NM_014481 |
| APH1A | NM_016022 |
| API5 | NM_006595 |
| APIP | NM_015957 |
| APOA1BP | NM_144772 |
| APOL2 | NM_030882 |
| APOOL | NM_198450 |
| APOPT1 | NM_032374 |
| APPL2 | NM_018171 |
| APTX | NM_175069 |
| ARAF | NM_001256197 |
| ARCN1 | NM_001655 |
| ARF1 | NM_001658 |
| ARF5 | NM_001662 |
| ARF6 | NM_001663 |
| ARFGAP2 | NM_032389 |
| ARFGAP3 | NM_014570 |
| ARFGEF2 | NM_006420 |
| ARFIP1 | NM_014447 |
| ARFIP2 | NM_012402 |
| ARFRP1 | NM_003224 |
| ARHGAP35 | NM_004491 |
| ARHGAP5 | NM_001173 |
| ARHGDIA | NM_004309 |
| ARHGEF10L | NM_018125 |
| ARUGEF11 | NM_014784 |
| ARHGEF40 | NM_018071 |
| ARIH1 | NM_005744 |
| ARIH2 | NM_006321 |
| ARIH2OS | NM_001123040 |
| ARL1 | NM_001177 |
| ARL14EP | NM_152315 |
| ARL5A | NM_012097 |
| ARL6IP4 | NM_016638 |
| ARL8A | NM_138795 |
| ARL8B | NM_018184 |
| ARMC1 | NM_018120 |
| ARMC1O | NM_031905 |
| ARMC5 | NM_024742 |
| ARMC6 | NM_033415 |
| ARMC7 | NM_024585 |
| ARMC8 | NM_014154 |
| ARMCX3 | NM_016607 |
| ARMCX5 | NM_022838 |
| ARNT | NM_001668 |
| ARPC1A | NM_006409 |
| ARPC2 | NM_005731 |
| ARPC5L | NM_030978 |
| ARV1 | NM_022786 |
| ASB1 | NM_001040445 |
| ASB6 | NM_001202403 |
| ASB7 | NM_198243 |
| ASB8 | NM_024095 |
| ASCC1 | NM_001198798 |
| ASCC3 | NM_006828 |
| ASF1A | NM_014034 |
| ASH2L | NM_004674 |
| ASNA1 | NM_004317 |
| ASNSD1 | NM_019043 |
| ASPSCR1 | NM_024083 |
| ASUN | NM_018164 |
| ASXL1 | NM_001164603 |
| ATAD1 | NM_032810 |
| ATAD3A | NM_018188 |
| ATE1 | NM_007041 |
| ATF1 | NM_005171 |
| ATF2 | NM_001880 |
| ATF4 | NM_001675 |
| ATF6 | NM_007348 |
| ATF7 | NM_001206682 |
| ATF7IP | NM_018179 |
| ATG12 | NM_004707 |
| ATG13 | NM_014741 |
| ATG16L1 | NM_017974 |
| ATG2A | NM_015104 |
| ATG2B | NM_018036 |
| ATG3 | NM_022488 |
| ATG4B | NM_013325 |
| ATG4D | NM_032885 |
| ATG5 | NM_004849 |
| ATG7 | NM_006395 |
| ATIC | NM_004044 |
| ATL2 | NM_022374 |
| ATMIM | NM_015251 |
| ATOX1 | NM_004045 |
| ATP2C1 | NM_014382 |
| ATP5A1 | NM_004045 |
| ATP5B | NM_001685 |
| ATP5C1 | NM_005174 |
| ATP5D | NM_001687 |
| ATP5F1 | NM_001688 |
| ATP5G2 | NM_005176 |
| ATP5G3 | NM_001689 |
| ATP5H | NM_006356 |
| ATP5J | NM_001685 |
| ATP5J2 | NM_004889 |
| ATP5J2-PTCD1 | NM_001198879 |
| ATP5L | NM_006476 |
| ATP5O | NM_001697 |
| ATP5S | NM_015684 |
| ATP5SL | NM_018035 |
| ATP6AP1 | NM_001183 |
| ATP6V0A2 | NM_012463 |
| ATP6V0B | NM_004047 |
| ATP6V0C | NM_001694 |
| ATP6V0D1 | NM_004691 |
| ATP6V0E1 | NM_003945 |
| ATP6V1C1 | NM_001695 |
| ATP6V1D | NM_015994 |
| ATP6V1E1 | NM_001696 |
| ATP6V1F | NM_004231 |
| ATP6V1G1 | NM_004888 |
| ATP6V1H | NM_015941 |
| ATPAF2 | NM_145691 |
| ATPIF1 | NM_016311 |
| ATRAID | NM_016085 |
| ATRN | NM_139321 |
| ATXN1O | NM_013236 |
| ATXN1L | NM_001137675 |
| ATXN2 | NM_002973 |
| ATXN2L | NM_007245 |
| ATXN7L3 | NM_020218 |
| ATXN7L3B | NM_001136262 |
| AUH | NM_001698 |
| AUP1 | NM_181575 |
| AURKAIP1 | NM_017900 |
| AXIN1 | NM_003502 |
| AZI2 | NM_022461 |
| AZIN1 | NM_015878 |
| B3GALT6 | NM_080605 |
| B4GALT3 | NM_003779 |
| B4GALT5 | NM_004776 |
| B4GALT7 | NM_007255 |
| BABAM1 | NM_014173 |
| BAD | NM_004322 |
| BAG1 | NM_004323 |
| BAG4 | NM_004874 |
| BAG6 | NM_004639 |
| BAHD1 | NM_014952 |
| BANF1 | NM_003860 |
| BAP1 | NM_004656 |
| BAZ1B | NM_032408 |
| BBS4 | NM_001252678 |
| BCAP29 | NM_018844 |
| BCAP31 | NM_005745 |
| BCAS2 | NM_005872 |
| BCAT2 | NM_001190 |
| BCCIP | NM_016567 |
| BCKDHA | NM_000709 |
| BCKDK | NM_005881 |
| BCL2L1 | NM_001191 |
| BCL2L13 | NM_015367 |
| BCL2L2-P | ABPN1 |
| | NM_001199864 |
| BCL7B | NM_001707 |
| BCLAF1 | NM_014739 |
| BCS1L | NM_004328 |
| BECN1 | NM_003766 |
| BFAR | NM_016561 |
| BIRC2 | NM_001166 |
| BIVM-ERCC5 | NM_001204425 |
| BLMH | NM_000386 |
| BLOC1S1 | NM_001487 |
| BLOC1S2 | NM_173809 |
| BLOC1S3 | NM_212550 |
| BLOC1S4 | NM_018366 |
| BLOC1S6 | NM_012388 |
| BLZP1 | NM_003666 |
| BMI1 | NM_005180 |
| BMS1 | NM_014753 |
| BNIP1 | NM_001205 |
| BNIP2 | NM_004330 |
| BOD1 | NM_138369 |
| BOLA1 | NM_016074 |
| BOLA3 | NM_001035505 |
| BPGM | NM_001724 |
| BPNT1 | NM_006085 |
| BPTF | NM_004459 |
| BRAT1 | NM_152743 |
| BRD2 | NM_005104 |
| BRD4 | NM_014299 |
| BRD7 | NM_013263 |
| BRD9 | NM_023924 |
| BRE | NM_004899 |
| BRF1 | NM_001519 |
| BRF2 | NM_018310 |
| BRIX1 | NM_018321 |
| BRK1 | NM_018462 |
| BRMS1 | NM_015399 |
| BRPF1 | NM_004634 |
| BRPF3 | NM_015695 |
| BSDC1 | NM_018045 |
| BSG | NM_001728 |
| BTBD2 | NM_017797 |
| BTD | NM_000060 |
| BTF3 | NM_001207 |
| BUB3 | NM_004725 |
| BZW1 | NM_014670 |
| C10orf12 | NM_015652 |
| C10orf2 | NM_021830 |
| C10orf76 | NM_024541 |
| C10orf88 | NM_024942 |
| C11orf1 | NM_022761 |
| C11orf24 | NM_022338 |
| C11orf31 | NM_170746 |
| C11orf57 | NM_018195 |
| C11orf58 | NM_014267 |
| C11orf73 | NM_016401 |
| C11orf83 | NM_001085372 |
| C12orf10 | NM_021640 |
| C12orf23 | NM_152261 |
| C12orf29 | NM_001009894 |
| C12orf44 | NM_021934 |
| C12orf45 | NM_152318 |
| C12orf5 | NM_020375 |
| C12orf52 | NM_032848 |
| C12orf57 | NM_138425 |
| C12orf65 | NM_152269 |
| C12orf66 | NM_152440 |
| C14orf1 | NM_007176 |
| C14orf119 | NM_017924 |
| C14orf142 | NM_032490 |
| C14orf166 | NM_016039 |
| C14orf2 | NM_004894 |
| C14orf28 | NM_001017923 |
| C15orf38- | NM_001199058 |
| AP3S2 | |
| C15orf57 | NM_052819 |
| C16orf13 | NM_032366 |
| C16orf62 | NM_020314 |
| C16orf72 | NM_014117 |
| C16orf91 | NM_001272051 |
| C17orf49 | NM_174893 |
| C17orf51 | NM_001113434 |
| C17orf58 | NM_181656 |
| C17orf59 | NM_017622 |
| C17orf70 | NM_025161 |
| C17orf85 | NM_001114118 |
| C18orf21 | NM_031446 |
| C18orf25 | NM_145055 |
| C18orf32 | NM_001035005 |
| C18orf8 | NM_013326 |
| C19orf43 | NM_024038 |
| C19orf53 | NM_014047 |
| C19orf60 | NM_001100418 |
| C19orf70 | NM_205767 |
| C1GALT1 | NM_020156 |
| C1QBP | NM_001212 |
| C1orf109 | NM_017850 |
| C1orf122 | NM_198446 |
| C1orf123 | NM_017887 |
| C1orf174 | NM_207356 |
| C1orf43 | NM_015449 |
| C1orf50 | NM_024097 |
| C1orf52 | NM_198077 |
| C20orf111 | NM_016470 |
| C20orf24 | NM_018840 |
| C21orf2 | NM_004928 |
| C21orf33 | NM_004649 |
| C21orf59 | NM_021254 |
| C22orf28 | NM_014306 |
| C22orf29 | NM_024627 |
| C22orf32 | NM_033318 |
| C2orf47 | NM_024520 |
| C2orf49 | NM_024093 |
| C2orf69 | NM_153689 |
| C2orf74 | NM_001143960 |
| C2orf76 | NM_001017927 |
| C3orf17 | NM_015412 |
| C3orf37 | NM_020187 |
| C3orf38 | NM_173824 |
| C3orf58 | NM_173552 |
| C4orf27 | NM_017867 |
| C4orf3 | NM_001001701 |
| C4orf52 | NM_001145432 |
| C5orf15 | NM_020199 |
| C5orf24 | NM_152409 |
| C6orf1 | NM_178508 |
| C6orf106 | NM_022758 |
| C6orf120 | NM_001029863 |
| C6orf136 | NM_145029 |
| C6orf226 | NM_001008739 |
| C6orf47 | NM_021184 |
| C6orf57 | NM_145267 |
| C6orf62 | NM_030939 |
| C6orf89 | NM_152734 |
| C7orf25 | NM_024054 |
| C7orf26 | NM_024067 |
| C7orf49 | NM_024033 |
| C7orf50 | NM_032350 |
| C7orf55 | NM_197964 |
| C7orf55- | LUC7L2 |
| | NM_001244584 |
| C7orf73 | NM_001130929 |
| C8orf33 | NM_023080 |
| C8orf40 | NM_138436 |
| C8orf59 | NM_001099670 |
| C8orf76 | NM_032847 |
| C8orf82 | NM_001001795 |
| C9orf123 | NM_033428 |
| C9orf16 | NM_024112 |
| C9orf37 | NM_032937 |
| C9orf64 | NM_032307 |
| C9orf69 | NM_152833 |
| C9orf78 | NM_016520 |
| C9orf89 | NM_032310 |
| CAB39 | NM_016289 |
| CALCOCO2 | NM_005831 |
| CALM1 | NM_006888 |
| CALR | NM_004343 |
| CALU | NM_001219 |
| CAMTA1 | NM_001195563 |
| CAMTA2 | NM_015099 |
| CANT1 | NM_138793 |
| CANX | NM_001746 |
| CAPN1 | NM_005186 |
| CAPN7 | NM_014296 |
| CAPNS1 | NM_001749 |
| CAPRIN1 | NM_005898 |
| CAPZA2 | NM_006135 |
| CAPZB | NM_004930 |
| CARKD | NM_018210 |
| CARS | NM_001751 |
| CARS2 | NM_024537 |
| CASC3 | NM_007359 |
| CASC4 | NM_138423 |
| CASP3 | NM_004345 |
| CASP7 | NM_001227 |
| CASP9 | NM_001229 |
| CBR4 | NM_032783 |
| CBX3 | NM_007276 |
| CBX5 | NM_012117 |
| CC2D1A | NM_017721 |
| CC2D1B | NM_032449 |
| CCAR1 | NM_018237 |
| CCBL1 | NM_001122672 |
| CCDC12 | NM_144715 |
| CCDC124 | NM_138442 |
| CCDC127 | NM_145265 |
| CCDC130 | NM_030818 |
| CCDC137 | NM_199287 |
| CCDC148 | NM_173463 |
| CCDC174 | NM_016474 |
| CCDC22 | NM_014008 |
| CCDC23 | NM_199342 |
| CCDC25 | NM_018245 |
| CCDC47 | NM_020198 |
| CCDC50 | NM_174908 |
| CCDC51 | NM_024661 |
| CCDC59 | NM_014167 |
| CCDC71 | NM_022903 |
| CCDC86 | NM_024098 |
| CCDC90A | NM_001031713 |
| CCDC92 | NM_025140 |
| CCDC94 | NM_018074 |
| CCM2 | NM_031443 |
| CCNB1IP1 | NM_021178 |
| CCNDBP1 | NM_012142 |
| CCNG1 | NM_004060 |
| CCNH | NM_001239 |
| CCNK | NM_001099402 |
| CCNL1 | NM_020307 |
| CCNL2 | NM_030937 |
| CCNY | NM_145012 |
| CCPG1 | NM_004748 |
| CCT3 | NM_005998 |
| CCT4 | NM_006430 |
| CCT5 | NM_012073 |
| CCT6A | NM_001762 |
| CCT7 | NM_006429 |
| CCT8 | NM_006585 |
| CD164 | NM_006016 |
| CD320 | NM_016579 |
| CD46 | NM_172350 |
| CD63 | NM_001780 |
| CD81 | NM_004355 |
| CCD82 | NM_002231 |
| CD99L2 | NM_031462 |
| CDC123 | NM_006023 |
| CDC16 | NM_003903 |
| CDC23 | NM_004661 |
| CDC27 | NM_001256 |
| CDC37 | NM_007065 |
| CDC37L1 | NM_017913 |
| CDC40 | NM_015891 |
| CDC42 | NM_001791 |
| CDC5L | NM_001253 |
| CDIP1 | NM_013399 |
| CDIPT | NM_006319 |
| CDK12 | NM_015083 |
| CDK13 | NM_003718 |
| CDK16 | NM_006201 |
| CDK2AP1 | NM_004642 |
| CDK4 | NM_000075 |
| CDK5RAP1 | NM_016082 |
| CDK8 | NM_001260 |
| CDK9 | NM_001261 |
| CDS2 | NM_003818 |
| CDV3 | NM_017548 |
| CDYL | NM_004824 |
| CEBPG | NM_001806 |
| CEBPZ | NM_005760 |
| CECR5 | NM_017829 |
| CELF1 | NM_006560 |
| CENPB | NM_001810 |
| CENPT | NM_025082 |
| CEP104 | NM_014704 |
| CEP57 | NM_001243777 |
| CEP63 | NM_025180 |
| CERK | NM_022766 |
| CERS2 | NM_022075 |
| CGGBP1 | NM_003663 |
| CHAMP1 | NM_032436 |
| CHCHD1 | NM_203298 |
| CHCHD2 | NM_016139 |
| CHCHD3 | NM_017812 |
| CHCHD4 | NM_144636 |
| CHCHD5 | NM_032309 |
| CHCHD7 | NM_024300 |
| CHD1L | NM_004284 |
| CHD4 | NM_001273 |
| CHD8 | NM_020920 |
| CHERP | NM_006387 |
| CHID1 | NM_023947 |
| CHKB | NM_005198 |
| CHMP1A | NM_002768 |
| CHMP2A | NM_014458 |
| CHMP2B | NM_014043 |
| CHMP4A | NM_014169 |
| CHMP4B | NM_176812 |
| CHMP5 | NM_016410 |
| CHMP6 | NM_024591 |
| CHP1 | NM_007236 |
| CHPT1 | NM_020244 |
| CHRAC1 | NM_017444 |
| CHST12 | NM_018641 |
| CHST7 | NM_019886 |
| CHTOP | NM_015607 |
| CHUK | NM_001278 |
| CHURC1 | NM_145165 |
| CHURC1-F | NTB |
| | NM_001202558 |
| CIAO1 | NM_004804 |
| CIB1 | NM_006384 |
| CIC | NM_015125 |
| CINP | NM_032630 |
| CIR1 | NM_004882 |
| CIRH1A | NM_032830 |
| CISD1 | NM_018464 |
| CISD2 | NM_001008388 |
| CISD3 | NM_001136498 |
| CKAP4 | NM_006825 |
| CLCC1 | NM_015127 |
| CLCN3 | NM_001829 |
| CLCN7 | NM_001287 |
| CLINT1 | NM_014666 |
| CLK3 | NM_003992 |
| CLNS1A | NM_001293 |
| CLOCK | NM_004898 |
| CLP1 | NM_006831 |
| CLPP | NM_006012 |
| CLPTM1 | NM_001294 |
| CLPTM1L | NM_030782 |
| CLPX | NM_006660 |
| CLTA | NM_001833 |
| CLTB | NM_001834 |
| CLTC | NM_004859 |
| CMAS | NM_018686 |
| CMC1 | NM_182523 |
| CMC2 | NM_020188 |
| CMC4 | NM_001018024 |
| CMPK1 | NM_016308 |
| CNBP | NM_003418 |
| CNIH | NM_005776 |
| CNIH4 | NM_014184 |
| CNNM2 | NM_199077 |
| CNNM3 | NM_017623 |
| CNOT1 | NM_016284 |
| CNOT11 | NM_017546 |
| CNOT2 | NM_014515 |
| CNOT3 | NM_014516 |
| CNOT4 | NM_013316 |
| CNOT7 | NM_013354 |
| CNST | NM_152609 |
| COA1 | NM_018224 |
| COA3 | NM_001040431 |
| COA4 | NM_016565 |
| COA5 | NM_001008215 |
| COA6 | NM_001012985 |
| COASY | NM_025233 |
| COG1 | NM_018714 |
| COG2 | NM_007357 |
| COG3 | NM_031431 |
| COG4 | NM_015386 |
| COG7 | NM_153603 |
| COG8 | NM_032382 |
| COMMD1 | NM_152516 |
| COMMD10 | NM_016144 |
| COMMD3 | NM_012071 |
| COMMD3-B | MI1 |
| | NM_001204062 |
| COMMD5 | NM_014066 |
| COMMD6 | NM_203495 |
| COMMD7 | NM_053041 |
| COMMD9 | NM_014186 |
| COMT | NM_000754 |
| COPA | NM_004371 |
| COPB1 | NM_016451 |
| COPB2 | NM_004766 |
| COPE | NM_007263 |
| COPG1 | NM_016128 |
| COPS2 | NM_004236 |
| COPS3 | NM_003653 |
| COPS4 | NM_016129 |
| COPS5 | NM_006837 |
| COPS6 | NM_006833 |
| COPS7A | NM_016319 |
| COPS7B | NM_022730 |
| COPS8 | NM_006710 |
| COPZ1 | NM_016057 |
| COQ10B | NM_025147 |
| COQ2 | NM_015697 |
| COQ4 | NM_016035 |
| COQ5 | NM_032314 |
| COQ6 | NM_182476 |
| CORO1C | NM_014325 |
| COX11 | NM_004375 |
| COX14 | NM_032901 |
| COX15 | NM_004376 |
| COX16 | NM_016468 |
| COX19 | NM_001031617 |
| COX20 | NM_198076 |
| COX411 | NM_001861 |
| COX5B | NM_001862 |
| COX6B1 | NM_001863 |
| COX6C | NM_004374 |
| COX7A2 | NM_001865 |
| COX7A2L | NM_004718 |
| COX7C | NM_001867 |
| COX8A | NM_004074 |
| CPD | NM_001304 |
| CPNE1 | NM_003915 |
| CPNE2 | NM_152727 |
| CPNE3 | NM_003909 |
| CPOX | NM_000097 |
| CPSF2 | NM_017437 |
| CPSF3L | NM_017871 |
| CPSF4 | NM_006693 |
| CPSF6 | NM_007007 |
| CPSF7 | NM_024811 |
| CRADD | NM_003805 |
| CRBM | NM_016302 |
| CRCP | NM_014478 |
| CREB3 | NM_006368 |
| CREBZF | NM_001039618 |
| CREG1 | NM_003851 |
| CRELD1 | NM_015513 |
| CRIPAK | NM_175918 |
| CRIPT | NM_014171 |
| CRK | NM_005206 |
| CRKL | NM_005207 |
| CRLS1 | NM_019095 |
| CRNKL1 | NM_016652 |
| CRTC2 | NM_181715 |
| CRY2 | NM_021117 |
| CSGALNACT2 | NM_018590 |
| CSNK1A1 | NM_001892 |
| CSNK1A1L | NM_145203 |
| CSNK1D | NM_001893 |
| CSNK1G3 | NM_004384 |
| CSNK2A3 | NM_001256686 |
| CSNK2B | NM_001320 |
| CSRP2BP | NM_020536 |
| CST3 | NM_000099 |
| CSTB | NM_000100 |
| CSTF1 | NM_001324 |
| CSTF2T | NM_015235 |
| CTAGE5 | NM_005930 |
| CTBP1 | NM_001328 |
| CTCF | NM_006565 |
| CTDSP2 | NM_005730 |
| CTNNA1 | NM_001903 |
| CTNNB1 | NM_001904 |
| CTNNBIP1 | NM_020248 |
| CTNNBL1 | NM_030877 |
| CTNND1 | NM_001085458 |
| CTSA | NM_000308 |
| CTSD | NM_001909 |
| CTTN | NM_005231 |
| CTU2 | NM_001012759 |
| CUEDC2 | NM_024040 |
| CUL1 | NM_003592 |
| CUL2 | NM_003591 |
| CUL4A | NM_003589 |
| CUL4B | NM_003588 |
| CUL5 | NM_003478 |
| CUTA | NM_015921 |
| CUX1 | NM_001913 |
| CWC15 | NM_016403 |
| CWC22 | NM_020943 |
| CWC25 | NM_017748 |
| CXXC1 | NM_014593 |
| CXXC5 | NM_016463 |
| CXorf40A | NM_178124 |
| CXorf40B | NM_001013845 |
| CXorf56 | NM_001170569 |
| CYB5B | NM_030579 |
| CYB5D2 | NM_001254755 |
| CYB5R3 | NM_000398 |
| CYC1 | NM_001916 |
| CYF1P1 | NM_014608 |
| CYHR1 | NM_138496 |
| CYP2U1 | NM_183075 |
| D2HGDH | NM_152783 |
| DAD1 | NM_001344 |
| DAG1 | NM_004393 |
| DAGLB | NM_139179 |
| DALRD3 | NM_018114 |
| DAP3 | NM_004632 |
| DARS | NM_001349 |
| DARS2 | NM_018122 |
| DAXX | NM_001350 |
| DAZAP1 | NM_018959 |
| DBT | NM_001918 |
| DCAF10 | NM_024345 |
| DCAF11 | NM_025230 |
| DCAF12 | NM_015397 |
| DCAF13 | NM_015420 |
| DCAF5 | NM_003861 |
| DCAF7 | NM_005828 |
| DCAF8 | NM_015726 |
| DCAKD | NM_024819 |
| DCTD | NM_001921 |
| DCTN2 | NM_006400 |
| DCTN3 | NM_007234 |
| DCTN4 | NM_016221 |
| DCTN5 | NM_032486 |
| DCTN6 | NM_006571 |
| DCTPP1 | NM_024096 |
| DCUN1D3 | NM_173475 |
| DCUN1D4 | NM_015115 |
| DCUN1D5 | NM_032299 |
| DDA1 | NM_024050 |
| DDB1 | NM_001923 |
| DDB2 | NM_000107 |
| DDOST | NM_005216 |
| DDRGK1 | NM_023935 |
| DDX1 | NM_004939 |
| DDX10 | NM_004398 |
| DDX17 | NM_006386 |
| DDX18 | NM_006773 |
| DDX19A | NM_018332 |
| DDX19B | NM_007242 |
| DDX21 | NM_004728 |
| DDX23 | NM_004818 |
| DDX24 | NM_020414 |
| DDX27 | NM_017895 |
| DDX39B | NM_004640 |
| DDX3X | NM_001356 |
| DDX41 | NM_016222 |
| DDX42 | NM_007372 |
| DDX46 | NM_014829 |
| DDX47 | NM_016355 |
| DDX49 | NM_019070 |
| DDX54 | NM_024072 |
| DDX56 | NM_019082 |
| DDX59 | NM_001031725 |
| DEDD | NM_032998 |
| DEF8 | NM_017702 |
| DEGS1 | NM_003676 |
| DEK | NM_003472 |
| DENND1A | NM_020946 |
| DENND4A | NM_005848 |
| DENR | NM_003677 |
| DERA | NM_015954 |
| DERL1 | NM_024295 |
| DERL2 | NM_016041 |
| DESI1 | NM_015704 |
| DEXI | NM_014015 |
| DFFA | NM_004401 |
| DGCR14 | NM_022719 |
| DGCR2 | NM_001173533 |
| DGCR6L | NM_033257 |
| DHPS | NM_001930 |
| DHRS12 | NM_024705 |
| DHRS7B | NM_015510 |
| DHX15 | NM_001358 |
| DHX16 | NM_003587 |
| DHX29 | NM_019030 |
| DHX30 | NM_014966 |
| DHX32 | NM_018180 |
| DHX33 | NM_020162 |
| DHX36 | NM_020865 |
| DHX38 | NM_014003 |
| DHX8 | NM_004941 |
| DHX9 | NM_001357 |
| DIABLO | NM_019887 |
| DIDO1 | NM_022105 |
| DIEXF | NM_014388 |
| DIMT1 | NM_014473 |
| DIRC2 | NM_032839 |
| DIS3 | NM_014953 |
| DIS3L2 | NM_152383 |
| DKC1 | NM_001363 |
| DLD | NM_000108 |
| DLG1 | NM_004087 |
| DLGAP4 | NM_183006 |
| DLST | NM_001933 |
| DMAP1 | NM_019100 |
| DNAAF2 | NM_018139 |
| DNAJA2 | NM_005880 |
| DNAJA3 | NM_005147 |
| DNAJB11 | NM_016306 |
| DNAJB12 | NM_017626 |
| DNAJB9 | NM_012328 |
| DNAJC10 | NM_018981 |
| DNAJC11 | NM_018198 |
| DNAJC14 | NM_032364 |
| DNAJC17 | NM_018163 |
| DNAJC19 | NM_145261 |
| DNAJC2 | NM_014377 |
| DNAJC21 | NM_194283 |
| DNAJC3 | NM_006260 |
| DNAJC4 | NM_005528 |
| DNAJC5 | NM_025219 |
| DNAJC7 | NM_003315 |
| DNAJC8 | NM_014280 |
| DNAJC9 | NM_015190 |
| DNASE2 | NM_001375 |
| DNLZ | NM_00108049 |
| DNM1L | NM_005690 |
| DNM2 | NM_004945 |
| DNTTIP1 | NM_052951 |
| DNTTIP2 | NM_014597 |
| DOHH | NM_031304 |
| DOLK | NM_014908 |
| DPAGT1 | NM_001382 |
| DPH1 | NM_001383 |
| DPH2 | NM_001384 |
| DPH3 | NM_206831 |
| DPH5 | NM_015958 |
| DPM1 | NM_003859 |
| DPP7 | NM_013379 |
| DPY30 | NM_032574 |
| DR1 | NM_001938 |
| DRAM2 | NM_178454 |
| DRAP1 | NM_006442 |
| DRG2 | NM_001388 |
| DROSHA | NM_013235 |
| DSCR3 | NM_006052 |
| DTWD1 | NM_020234 |
| DUSP11 | NM_003584 |
| DUSP14 | NM_007026 |
| DUSP16 | NM_030640 |
| DUSP22 | NM_020185 |
| DUT | NM_001948 |
| DVL3 | NM_004423 |
| DYM | NM_017653 |
| DYNC1LI1 | NM_016141 |
| DYNLL2 | NM_080677 |
| DYNLRB1 | NM_014183 |
| DYNLT1 | NM_006519 |
| E2F4 | NM_001950 |
| E4F1 | NM_004424 |
| EAF1 | NM_033083 |
| EAPP | NM_018453 |
| EARS2 | NM_001083614 |
| EBAG9 | NM_004215 |
| EBNA1BP2 | NM_006824 |
| ECD | NM_007265 |
| ECH1 | NM_001398 |
| ECHDC1 | NM_001002030 |
| ECHS1 | NM_004092 |
| ECI1 | NM_001919 |
| ECI2 | NM_006117 |
| ECSIT | NM_016581 |
| EDC3 | NM_025083 |
| EDC4 | NM_014329 |
| EDEM3 | NM_025191 |
| EDF1 | NM_003792 |
| EED | NM_003797 |
| EEF1B2 | NM_001959 |
| EEF1E1 | NM_004280 |
| EEF2 | NM_001961 |
| EEFSEC | NM_021937 |
| EFCAB14 | NM_014774 |
| EFHA1 | NM_152726 |
| EFR3A | NM_015137 |
| EFTUD1 | NM_024580 |
| EFTUD2 | NM_004247 |
| EGLN2 | NM_053046 |
| EHMT1 | NM_024757 |
| EI24 | NM_004879 |
| EID2 | NM_153232 |
| EIF1 | NM_005801 |
| EIF1AD | NM_032325 |
| EIF1B | NM_005875 |
| EIF2A | NM_032025 |
| EIF2AK1 | NM_014413 |
| EIF2AK3 | NM_004836 |
| EIF2AK4 | NM_001013702 |
| EIF2B2 | NM_014239 |
| EIF2B3 | NM_020365 |
| EIF2B4 | NM_015636 |
| EIF2B5 | NM_003907 |
| EIF2D | NM_006893 |
| EIF2S1 | NM_004094 |
| EIF2S2 | NM_003908 |
| EIF3A | NM_003750 |
| EIF3B | NM_003751 |
| EIF3D | NM_003753 |
| EIF3E | NM_001568 |
| EIF3G | NM_003755 |
| EIF3H | NM_003756 |
| EIF3I | NM_003757 |
| EIF3J | NM_003758 |
| EIF3K | NM_013234 |
| EIF3L | NM_016091 |
| EIF3M | NM_006360 |
| EIF4A1 | NM_001416 |
| EIF4A3 | NM_014740 |
| EIF4E2 | NM_001846 |
| EIF4G1 | NM_004953 |
| EIF4G2 | NM_001418 |
| EIF4G3 | NM_003760 |
| EIF4H | NM_022170 |
| EIF5 | NM_001969 |
| EIF5A | NM_001970 |
| EIF5AL1 | NM_001099692 |
| EIF5B | NM_015904 |
| EIF6 | NM_002212 |
| ELAC2 | NM_018127 |
| ELAVL1 | NM_001419 |
| ELF2 | NM_006874 |
| ELK1 | NM_005229 |
| ELK4 | NM_001973 |
| ELL2 | NM_012081 |
| ELMOD3 | NM_032213 |
| ELOVL1 | NM_022821 |
| ELP2 | NM_018255 |
| ELP3 | NM_018091 |
| ELP4 | NM_019040 |
| ELP6 | NM_001031703 |
| EMC1 | NM_015047 |
| EMC10 | NM_175063 |
| EMC2 | NM_014673 |
| EMC3 | NM_018447 |
| EMC4 | NM_016454 |
| EMC6 | NM_031298 |
| EMC7 | NM_020154 |
| EMC8 | NM_006067 |
| EMC9 | NM_016049 |
| EMD | NM_000117 |
| EMG1 | NM_006331 |
| ENDOG | NM_004435 |
| ENOPH1 | NM_021204 |
| ENSA | NM_004436 |
| ENTPD4 | NM_004901 |
| ENTPD6 | NM_001247 |
| ENY2 | NM_020189 |
| EPC1 | NM_025209 |
| EPM2AIP1 | NM_014805 |
| EPN1 | NM_013333 |
| EPRS | NM_004446 |
| ERAL1 | NM_005702 |
| ERAP1 | NM_016442 |
| ERCC1 | NM_001983 |
| ERCC2 | NM_000400 |
| ERCC3 | NM_000122 |
| ERCC5 | NM_000123 |
| ERGIC2 | NM_016570 |
| ERGIC3 | NM_015966 |
| ERH | NM_004450 |
| ERI3 | NM_024066 |
| ERICH1 | NM_207332 |
| ERLEC1 | NM_015701 |
| ERO1L | NM_014584 |
| ERP44 | NM_015051 |
| ESD | NM_001984 |
| ESF1 | NM_016649 |
| ETF1 | NM_004730 |
| ETFA | NM_000126 |
| ETFB | NM_001985 |
| ETV6 | NM_001987 |
| EWSR1 | NM_005243 |
| EXD2 | NM_018199 |
| EXOC1 | NM_018261 |
| EXOC2 | NM_018303 |
| EXOC3 | NM_007277 |
| EXOC4 | NM_021807 |
| EXOC7 | NM_015219 |
| EXOC8 | NM_175876 |
| EXOSC1 | NM_016046 |
| EXOSC10 | NM_002685 |
| EXOSC2 | NM_014285 |
| EXOSC4 | NM_019037 |
| EXOSC7 | NM_015004 |
| EXOSC8 | NM_181503 |
| EXT2 | NM_207122 |
| EXTL3 | NM_0011440 |
| FADD | NM_003824 |
| FAF1 | NM_007051 |
| FAF2 | NM_014613 |
| FAHD1 | NM_031208 |
| FAM104B | NM_138362 |
| FAM108A1 | NM_031213 |
| FAM108B1 | NM_016014 |
| FAM114A2 | NM_018691 |
| FAM118B | NM_024556 |
| FAM120A | NM_014612 |
| FAM120AOS | NM_198841 |
| FAM120B | NM_032448 |
| FAM122A | NM_138333 |
| FAM127B | NM_001078172 |
| FAM134A | NM_024293 |
| FAM134C | NM_178126 |
| FAM136A | NM_032822 |
| FAM149B1 | NM_173348 |
| FAM160A2 | NM_032127 |
| FAM160B1 | NM_020940 |
| FAM160B2 | NM_022749 |
| FAM162A | NM_014367 |
| FAM168B | NM_001009993 |
| FAM173A | NM_023933 |
| FAM173B | NM_001258388 |
| FAM174A | NM_198507 |
| FAM175B | NM_032182 |
| FAM177A1 | NM_173607 |
| FAM178A | NM_001243770 |
| FAM192A | NM_024946 |
| FAM199X | NM_207318 |
| FAM200A | NM_145111 |
| FAM204A | NM_022063 |
| FAM206A | NM_017832 |
| FAM208B | NM_017782 |
| FAM20B | NM_014864 |
| FAM210B | NM_080821 |
| FAM32A | NM_014077 |
| FAM35A | NM_019054 |
| FAM3A | NM_021806 |
| FAM50A | NM_004699 |
| FAM50B | NM_012135 |
| FAM58A | NM_152274 |
| FAM63A | NM_018379 |
| FAM73B | NM_032809 |
| FAM8A1 | NM_016255 |
| FAM96A | NM_032231 |
| FAM96B | NM_016062 |
| FAM98A | NM_015475 |
| FARS2 | NM_006567 |
| FARSA | NM_004461 |
| FARSB | NM_005687 |
| FASTK | NM_006712 |
| FASTKD2 | NM_014929 |
| FASTKD5 | NM_021826 |
| FBRSL1 | NM_001142641 |
| FBXL15 | NM_024326 |
| FBXL17 | NM_001163315 |
| FBXL3 | NM_012158 |
| FBXL4 | NM_012160 |
| FBXL5 | NM_012161 |
| FBXL6 | NM_012162 |
| FBXO11 | NM_025133 |
| FBXO18 | NM_032807 |
| FBXO22 | NM_012170 |
| FBXO28 | NM_015176 |
| FBXO3 | NM_012175 |
| FBXO38 | NM_030793 |
| FBXO42 | NM_018994 |
| FBXO45 | NM_001105573 |
| FBXO6 | NM_018438 |
| FBXO7 | NM_012179 |
| FBXW11 | NM_012300 |
| FBXW2 | NM_012164 |
| FBXW4 | NM_022039 |
| FBXW5 | NM_018998 |
| FBXW7 | NM_001257069 |
| FCF1 | NM_015962 |
| FDFT1 | NM_004462 |
| FDPS | NM_002004 |
| FDX1 | NM_004109 |
| FECH | NM_000140 |
| FEM1C | NM_020177 |
| FEN1 | NM_004111 |
| FEZ2 | NM_005102 |
| FGFR1OP2 | NM_015633 |
| FH | NM_000143 |
| FIBP | NM_004214 |
| FICD | NM_007076 |
| FIP1L1 | NM_030917 |
| FIS1 | NM_016068 |
| FIZ1 | NM_032836 |
| FKBP3 | NM_002013 |
| FKBP8 | NM_012181 |
| FKBPL | NM_022110 |
| FKRP | NM_024301 |
| FLAD1 | NM_025207 |
| FLCN | NM_144606 |
| FLOT1 | NM_005803 |
| FLOT2 | NM_004475 |
| FNDC3A | NM_014923 |
| FNTA | NM_002027 |
| FNTB | NM_002028 |
| FOPNL | NM_144600 |
| FOXK2 | NM_004514 |
| FOXP4 | NM_138457 |
| FOXRED1 | NM_017547 |
| FPGS | NM_004957 |
| FPGT | NM_003833 |
| FRA10AC1 | NM_145246 |
| FTO | NM_001080432 |
| FTSJ1 | NM_012280 |
| FTSJ2 | NM_013393 |
| FTSJ3 | NM_017647 |
| FTSJD1 | NM_018348 |
| FTSJD2 | NM_015050 |
| FUBP1 | NM_003902 |
| FUK | NM_145059 |
| FUNDC2 | NM_023934 |
| FXN | NM_000144 |
| FYTTD1 | NM_032288 |
| FZR1 | NM_016263 |
| G3BP1 | NM_005754 |
| GAA | NM_000152 |
| GABARAP | NM_007278 |
| GABARAPL2 | NM_007285 |
| GABPB1 | NM_002041 |
| GADD45GIP1 | NM_052850 |
| GALK2 | NM_002044 |
| GALNS | NM_000512 |
| GALNT1 | NM_020474 |
| GALNT2 | NM_004481 |
| GALT | NM_000155 |
| GANAB | NM_198334 |
| GAPVD1 | NM_015635 |
| GARS | NM_002047 |
| GART | NM_000819 |
| GATAD2A | NM_017660 |
| GATAD2B | NM_020699 |
| GATC | NM_176818 |
| GBA | NM_000157 |
| GBA2 | NM_020944 |
| GBF1 | NM_004193 |
| GCC1 | NM_024523 |
| GCDH | NM_000159 |
| GCLC | NM_001498 |
| GCLM | NM_002061 |
| GDE1 | NM_016641 |
| GDI2 | NM_001494 |
| GDPGP1 | NM_001013657 |
| GEMIN7 | NM_024707 |
| GEMIN8 | NM_017856 |
| GET4 | NM_015949 |
| GFER | NM_005262 |
| GFM1 | NM_024996 |
| GFOD2 | NM_001243650 |
| GGCT | NM_001199816 |
| GGNBP2 | NM_024835 |
| GGI7 | NM_178025 |
| GHDC | NM_001142623 |
| GHITM | NM_014394 |
| GID8 | NM_017896 |
| GINM1 | NM_138785 |
| GIPC1 | NM_005716 |
| GLCE | NM_015554 |
| GLE1 | NM_001499 |
| GLG1 | NM_012201 |
| GL14 | NM_138465 |
| GLO1 | NM_006708 |
| GLRX2 | NM_001243399 |
| GLRX3 | NM_006541 |
| GLRX5 | NM_016417 |
| GLT8D1 | NM_018446 |
| GLTP | NM_016433 |
| GLTPD1 | NM_001029885 |
| GLYR1 | NM_032569 |
| GMPPA | NM_013335 |
| GMPR2 | NM_016576 |
| GNB1 | NM_002074 |
| BNB2 | NM_005273 |
| GNE | NM_005476 |
| GNL2 | NM_013285 |
| GNL3 | NM_014366 |
| GNPAT | NM_014236 |
| GNPDA1 | NM_005471 |
| GNPNAT1 | NM_198066 |
| GNPTG | NM_032520 |
| GNS | NM_002076 |
| GOLGA1 | NM_002077 |
| GOLGA2 | NM_004486 |
| GOLGA3 | NM_005895 |
| GOLGA5 | NM_005113 |
| GOLGA7 | NM_016099 |
| GOLGB1 | NM_004487 |
| GOLPH3 | NM_022130 |
| GOLT1B | NM_016072 |
| GOPC | NM_020399 |
| GORASP1 | NM_031899 |
| GORASP2 | NM_015530 |
| GOSR1 | NM_004871 |
| GOSR2 | NM_004287 |
| GPAA1 | NM_003801 |
| GPANK1 | NM_033177 |
| GPATCH4 | NM_015590 |
| GPBP1 | NM_022913 |
| GPBP1L1 | NM_021639 |
| GPHN | NM_020806 |
| GPT | NM_000175 |
| GPKOW | NM_015698 |
| GPN1 | NM_007266 |
| GPN2 | NM_018066 |
| GPN3 | NM_016301 |
| GPR107 | NM_020960 |
| GPR108 | NM_001080452 |
| GPS1 | NM_004127 |
| GPS2 | NM_004489 |
| GPX4 | NM_002085 |
| GRAMD4 | NM_015124 |
| GRHPR | NM_012203 |
| GRINA | NM_000837 |
| GRIPAP1 | NM_020137 |
| GRPEL1 | NM_025196 |
| GRSF1 | NM_002092 |
| GRWD1 | NM_031485 |
| GSK3A | NM_019884 |
| GSK3B | NM_002093 |
| GSPT1 | NM_002094 |
| GSPT2 | NM_018094 |
| GSR | NM_000637 |
| GSS | NM_000178 |
| GSTK1 | NM_001143681 |
| GSTM4 | NM_000850 |
| GSTO1 | NM_004832 |
| GTDC2 | NM_032806 |
| GTF2A1 | NM_015859 |
| GTF2B | NM_001514 |
| GTF2F1 | NM_002096 |
| GTF2F2 | NM_004128 |
| GTF2H1 | NM_005316 |
| GTF2H4 | NM_001517 |
| GTF2H5 | NM_207118 |
| GTF2I | NM_001518 |
| GTF3A | NM_002097 |
| GTF3C1 | NM_001520 |
| GTF3C2 | NM_001521 |
| GTF3C3 | NM_012086 |
| GTF3C5 | NM_012087 |
| GTF3C6 | NM_138408 |
| GTPBP10 | NM_033107 |
| GTPBP4 | NM_012341 |
| GTPBP5 | NM_015666 |
| GTPBP8 | NM_014170 |
| GUK1 | NM_000858 |
| GZF1 | NM_011482 |
| H1FX | NM_006026 |
| H2AFV | NM_012412 |
| H2AFX | NM_002105 |
| H2AFY | NM_004893 |
| H2AFZ | NM_002106 |
| HADH | NM_005327 |
| HADHA | NM_000182 |
| HAGH | NM_005326 |
| HARS | NM_002109 |
| HARS2 | NM_012208 |
| HAT1 | NM_003642 |
| HAUS3 | NM_024511 |
| HAUS4 | NM_017815 |
| HAUS7 | NM_017518 |
| HAX1 | NM_006118 |
| HBP1 | NM_012257 |
| HBS1L | NM_006620 |
| HCCS | NM_005333 |
| HCFC1 | NM_005334 |
| HDAC2 | NM_001527 |
| HDAC3 | NM_003883 |
| HDAC6 | NM_006044 |
| HDAC8 | NM_018486 |
| HDDC3 | NM_198527 |
| HDGF | NM_004494 |
| HDHD3 | NM_031219 |
| HDLBP | NM_005336 |
| HEATR2 | NM_017802 |
| HEATR5A | NM_015473 |
| HEBP1 | NM_015987 |
| HECTD3 | NM_024602 |
| HELZ | NM_014877 |
| HEMK1 | NM_016173 |
| HERC4 | NM_015601 |
| HERPUD1 | NM_014685 |
| HERPUD2 | NM_022373 |
| HEXA | NM_000520 |
| HEXDC | NM_173620 |
| HEXIM1 | NM_006460 |
| HGS | NM_004712 |
| HIAT1 | NM_033055 |
| HIATL1 | NM_032558 |
| HIBADH | NM_152740 |
| HIGD1A | NM_014056 |
| HIGD2A | NM_138820 |
| HINFP | NM_015517 |
| HINT1 | NM_005340 |
| HINT2 | NM_032593 |
| HIST1H2BC | NM_003526 |
| HIVEP1 | NM_002114 |
| HMBS | NM_000190 |
| HMG20A | NM_018200 |
| HMG20B | NM_006339 |
| HMGB1 | NM_002128 |
| HMGN3 | NM_004242 |
| HMGXB3 | NM_014983 |
| HMGXB4 | NM_001003681 |
| HMOX2 | NM_002134 |
| HNIL | NM_144570 |
| HNRNPA0 | NM_006805 |
| HNRNPA2B1 | NM_002137 |
| HNRNPAB | NM_004499 |
| HNRNPC | NM_004500 |
| HNRNPD | NM_002138 |
| HNRNPF | NM_004966 |
| HNRNPH1 | NM_005520 |
| HNRNPH2 | NM_019597 |
| HNRNPK | NM_002140 |
| HNRNPL | NM_001533 |
| HNRNPM | NM_005968 |
| HNRNPR | NM_005826 |
| HNRNPU | NM_004501 |
| HNRNPUL1 | NM_007040 |
| HNRNPUL2 | NM_001079559 |
| HNRPDL | NM_031372 |
| HNRPLL | NM_138394 |
| HP1BP3 | NM_016287 |
| HPS1 | NM_000195 |
| HPS6 | NM_024747 |
| HS1BP3 | NM_022460 |
| HS2ST1 | NM_012262 |
| HS6ST1 | NM_004807 |
| HSBP1 | NM_001537 |
| HSCB | NM_172002 |
| HSD17B10 | NM_004493 |
| HSD17B12 | NM_016142 |
| HSD17B4 | NM_000414 |
| HSPA14 | NM_016299 |
| HSPA4 | NM_002154 |
| HSPA5 | NM_005347 |
| HSPA8 | NM_006597 |
| HSPA9 | NM_004134 |
| HSPBP1 | NM_012267 |
| HSPE1-MOB4 | NM_001202485 |
| HTATIP2 | NM_006410 |
| HTRA2 | NM_013247 |
| HTT | NM_002111 |
| HUS1 | NM_004507 |
| HUWE1 | NM_031407 |
| HYOU1 | NM_006389 |
| HYPK | NM_016400 |
| IAH1 | NM_001039613 |
| IARS | NM_002161 |
| IARS2 | NM_018060 |
| IBA57 | NM_001010867 |
| IBTK | NM_015525 |
| ICK | NM_014920 |
| ICMT | NM_012405 |
| ICT1 | NM_001545 |
| IDE | NM_001165946 |
| IDH3A | NM_005530 |
| IDH3B | NM_006899 |
| IDH3G | NM_004135 |
| IDI1 | NM_004508 |
| IER31P1 | NM_016097 |
| IFNAR1 | NM_000629 |
| IFNGR1 | NM_000416 |
| IFRD1 | NM_001550 |
| IFT27 | NM_006860 |
| IKZF5 | NM_001271840 |
| IL13RA1 | NM_001560 |
| IL6ST | NM_002184 |
| ILF2 | NM_004515 |
| ILKAP | NM_030768 |
| ILVBL | NM_006844 |
| IMMT | NM_006839 |
| IMP3 | NM_018285 |
| IMP4 | NM_033416 |
| IMPAD1 | NM_017813 |
| INF2 | NM_022489 |
| ING1 | NM_198217 |
| INO80B | NM_031288 |
| INO80E | NM_173618 |
| INPP5A | NM_005539 |
| INPP5K | NM_016532 |
| INSIG2 | NM_016133 |
| INTS1 | NM_001080453 |
| INTS10 | NM_018142 |
| INTS12 | NM_020395 |
| INTS3 | NM_023015 |
| INTS4 | NM_033547 |
| INVS | NM_014425 |
| IP6K1 | NM_153273 |
| IP6K2 | NM_001190316 |
| IP07 | NM_006391 |
| IP08 | NM_006390 |
| IP09 | NM_018085 |
| IRAK1 | NM_001569 |
| IREB2 | NM_004136 |
| IRF2BP1 | NM_015649 |
| IRF2BP2 | NM_182972 |
| IRF2BPL | NM_024496 |
| IRGQ | NM_001007561 |
| ISCU | NM_014301 |
| ISOC2 | NM_024710 |
| IST1 | NM_001270977 |
| ISY1 | NM_020701 |
| ISY1-RAB43 | NM_001204890 |
| ITCH | NM_031483 |
| ITFG1 | NM_030790 |
| ITFG3 | NM_032039 |
| ITGB1 | NM_002211 |
| ITGB1BP1 | NM_022334 |
| ITM2B | NM_021999 |
| ITPA | NM_033453 |
| ITPK1 | NM_014216 |
| ITPKC | NM_025194 |
| ITPRIPL2 | NM_001034841 |
| IVNS1ABP | NM_006469 |
| IWS1 | NM_017969 |
| JAGN1 | NM_032492 |
| JAK1 | NM_002227 |
| JKAMP | NM_016475 |
| JMJD4 | NM_023007 |
| JMJD6 | NM_015167 |
| JMJD7 | NM_001114632 |
| JMJD8 | NM_001005920 |
| JOSD2 | NM_138334 |
| JTB | NM_006694 |
| JUND | NM_005354 |
| KANSL2 | NM_017822 |
| KANSL3 | NM_001115016 |
| KARS | NM_005548 |
| KAT2B | NM_003884 |
| KAT5 | NM_006388 |
| KAT8 | NM_032188 |
| KBTBD2 | NM_015483 |
| KBTBD4 | NM_016506 |
| KBTBD7 | NM_032138 |
| KCMF1 | NM_020122 |
| KCTD20 | NM_173562 |
| KCTD21 | NM_001029859 |
| KCTD6 | NM_153331 |
| KDM2A | NM_012308 |
| KDM4A | NM_014663 |
| KDM5C | NM_004187 |
| KDSR | NM_002035 |
| KHDRBS1 | NM_006559 |
| KHNYN | NM_015299 |
| KHSRP | NM_003685 |
| KIAA0100 | NM_014680 |
| KIAA0141 | NM_014773 |
| KIAA0195 | NM_014738 |
| KIAA0196 | NM_014846 |
| KIAA0232 | NM_014743 |
| KIAA0319L | NM_024874 |
| KIAA0391 | NM_014672 |
| KIAA0754 | NM_015038 |
| KIAA0947 | NM_015325 |
| KIAA1143 | NM_020696 |
| KIAA1191 | NM_020444 |
| KIAA1429 | NM_015496 |
| KIAA1430 | NM_020827 |
| KIAA1586 | NM_020931 |
| KIAA1704 | NM_018559 |
| KIAA1715 | NM_030650 |
| KIAA1919 | NM_153369 |
| KIAA1967 | NM_021174 |
| KIAA2013 | NM_138346 |
| KLC4 | NM_138343 |
| HLP3 | NM_016531 |
| FLP9 | NM_001206 |
| KLHDC2 | NM_014315 |
| KLHDC3 | NM_057161 |
| KLHL20 | NM_014458 |
| KLHL25 | NM_022480 |
| KLHL36 | NM_024731 |
| KLHL5 | NM_015990 |
| KLHL8 | NM_020803 |
| KPNA1 | NM_002264 |
| KPNB1 | NM_002265 |
| KRCC1 | NM_016618 |
| KRR1 | NM_007043 |
| KTI12 | NM_138417 |
| KTN1 | NM_004986 |
| KXD1 | NM_024069 |
| L3MBTL2 | NM_031488 |
| LACTB | NM_032857 |
| LAGE3 | NM_006014 |
| LAMP1 | NM_005561 |
| LAMP2 | NM_002294 |
| LAMTOR1 | NM_017907 |
| LAMTOR2 | NM_014017 |
| LAMTOR3 | NM_021970 |
| LAMTOR4 | NM_001008395 |
| LAMTOR5 | NM_006402 |
| LAP3 | NM_015907 |
| LAPTM4A | NM_014713 |
| LARP1 | NM_015315 |
| LARP4 | NM_052879 |
| LARP7 | NM_015454 |
| LARS2 | NM_015340 |
| LCOR | NM_032440 |
| LDHA | NM_005566 |
| LEND2 | NM_181336 |
| LENG1 | NM_024316 |
| LEPROT | NM_017526 |
| LETM1 | NM_012318 |
| LETMD1 | NM_015416 |
| LGALSL | NM_014181 |
| LHPP | NM_022126 |
| LIAS | NM_006859 |
| LIG3 | NM_002311 |
| LIG4 | NM_002312 |
| LIN37 | NM_019104 |
| LIN54 | NM_001115007 |
| LIN7C | NM_018362 |
| LINS | NM_001040616 |
| LIPT1 | NM_015929 |
| LMAN1 | NM_005570 |
| LMBRD1 | NM_018368 |
| LMF2 | NM_033200 |
| LMO4 | NM_006769 |
| LNX2 | NM_153371 |
| LOC100129361 | NM_001271592 |
| LOC100289561 | NM_001242713 |
| LOC441155 | NM_001271675 |
| LOC729020 | NM_001143909 |
| LONP1 | NM_004793 |
| LONP2 | NM_031490 |
| LPCAT3 | NM_005768 |
| LPIN1 | NM_145693 |
| LPPR2 | NM_022737 |
| LRFN3 | NM_024509 |
| LRPAP1 | NM_002337 |
| LRPPRC | NM_133259 |
| LRRC14 | NM_014665 |
| LRRC24 | NM_001024678 |
| LRRC28 | NM_144598 |
| LRRC40 | NM_017768 |
| LRRC41 | NM_006369 |
| LRRC42 | NM_052940 |
| LRRC47 | NM_020710 |
| LRRC57 | NM_153260 |
| LRRC59 | NM_018509 |
| LRRC8A | NM_019594 |
| LRRFIP2 | NM_017724 |
| LRSAM1 | NM_138361 |
| LSG1 | NM_018385 |
| LSM1 | NM_014462 |
| LSM10 | NM_032881 |
| LSM14A | NM_015578 |
| LSM14B | NM_144703 |
| LSM2 | NM_021177 |
| LSM3 | NM_014463 |
| LSM4 | NM_012321 |
| LSM5 | NM_012322 |
| LSM6 | NM_007080 |
| LSM7 | NM_016199 |
| LSMD1 | NM_032356 |
| LSS | NM_002340 |
| LTV1 | NM_032860 |
| LUC7L2 | NM_016019 |
| LUC7L3 | NM_006107 |
| LUZP6 | NM_001128619 |
| LYRM1 | NM_020424 |
| LYRM4 | NM_020408 |
| LYRM5 | NM_001001660 |
| LYSMD1 | NM_212551 |
| LYSMD3 | NM_198273 |
| LYSMD4 | NM_152449 |
| LZTR1 | NM_006767 |
| M6PR | NM_002355 |
| MAD2L1BP | NM_014628 |
| MAD2L2 | NM_006341 |
| MAEA | NM_005882 |
| MAGED1 | NM_006986 |
| MAGEF1 | NM_022149 |
| MAGOH | NM_002370 |
| MAGT1 | NM_032121 |
| MAK16 | NM_032509 |
| MALSU1 | NM_138446 |
| MAN1A2 | NM_006699 |
| MAN1B1 | NM_016219 |
| MAN2A2 | NM_006122 |
| MAN2B2 | NM_015274 |
| MAN2C1 | NM_006715 |
| MAP1LC3B2 | NM_001085481 |
| MAP2K1 | NM_002755 |
| MAP2K2 | NM_030662 |
| MAP2K5 | NM_002757 |
| MAP3K7 | NM_003188 |
| MAP4K4 | NM_004834 |
| MAPK1 | NM_002745 |
| MAPK1IP1L | NM_144578 |
| MAPK6 | NM_002748 |
| MAPK8 | NM_002750 |
| MAPK9 | NM_002752 |
| MAPKAP1 | NM_024117 |
| MAPKAPK2 | NM_004759 |
| MAPKAPK5 | NM_003668 |
| MAPRE2 | NM_014268 |
| 02-Mar | NM_016496 |
| 05-Mar | NM_017824 |
| 06-Mar | NM_005885 |
| 07-Mar | NM_022826 |
| MARK3 | NM_002376 |
| MARK1 | NM_031417 |
| MARS | NM_004990 |
| MARS2 | NM_138395 |
| MAT2B | NM_013283 |
| MAVS | NM_001206491 |
| MAX | NM_002382 |
| MAZ | NM_002383 |
| MBD1 | NM_002384 |
| MBD2 | NM_003927 |
| MBD3 | NM_003926 |
| MBD4 | NM_003925 |
| MBLAC1 | NM_203397 |
| MBNL2 | NM_144778 |
| MBTPS1 | NM_003791 |
| MBTPS2 | NM_015884 |
| MCAT | NM_014507 |
| MCCC1 | NM_020166 |
| MCEE | NM_032601 |
| MCFD2 | NM_139279 |
| MCM3AP | NM_003906 |
| MCM7 | NM_005916 |
| MCMBP | NM_024834 |
| MCOLN1 | NM_020533 |
| MCPH1 | NM_001172574 |
| MCRS1 | NM_006337 |
| MCTS1 | NM_014060 |
| MCU | NM_001270680 |
| MDC1 | NM_014641 |
| MDP1 | NM_138476 |
| ME2 | NM_002396 |
| MEAF6 | NM_022756 |
| MECP2 | NM_004992 |
| MED10 | NM_032286 |
| MED11 | NM_001001683 |
| MED13 | NM_005121 |
| MED14 | NM_004229 |
| MED16 | NM_005481 |
| MED19 | NM_153450 |
| MED20 | NM_004275 |
| MED21 | NM_004264 |
| MED24 | NM_014815 |
| MED29 | NM_017592 |
| MED31 | NM_016060 |
| MED4 | NM_014166 |
| MED6 | NM_005466 |
| MED7 | NM_004270 |
| MED8 | NM_052877 |
| MEF2A | NM_005587 |
| MEF2BNB | NM_001145783 |
| MEMO1 | NM_015955 |
| MEN1 | NM_000244 |
| MEPCE | NM_019606 |
| METAP1 | NM_015143 |
| METAP2 | NM_006838 |
| METRN | NM_024042 |
| METTL13 | NM_014955 |
| METTL14 | NM_020961 |
| METTL16 | NM_024086 |
| METTL17 | NM_022734 |
| METTL18 | NM_033418 |
| METTL20 | NM_173802 |
| METTL21A | NM_145280 |
| METTL23 | NM_001080510 |
| METTL2A | NM_181725 |
| METTL2B | NM_018396 |
| METTL3 | NM_019852 |
| METTL5 | NM_014168 |
| MFAP1 | NM_005926 |
| MFAP3 | NM_005927 |
| MFF | NM_020194 |
| MFN1 | NM_033540 |
| MFSD11 | NM_024311 |
| MFSD12 | NM_174983 |
| MFSD3 | NM_138431 |
| MFSD5 | NM_032889 |
| MGAT2 | NM_002408 |
| MGAT4B | NM_014275 |
| MGNE1 | NM_052865 |
| MGNT | NM_002412 |
| MGRN1 | NM_015246 |
| MGST3 | NM_004528 |
| MIA3 | NM_198551 |
| MIB1 | NM_020774 |
| MICALL1 | NM_033386 |
| MICU1 | NM_006077 |
| MID1IP1 | NM_021242 |
| MIDN | NM_177401 |
| MIEN1 | NM_032339 |
| MIER1 | NM_001077701 |
| MIF | NM_002415 |
| MIF4GD | NM_020679 |
| MI1P | NM_021933 |
| MINOS1 | NM_001032363 |
| MIS12 | NM_024039 |
| MITD1 | NM_138798 |
| MKI67IP | NM_032390 |
| MKKS | NM_018848 |
| MKLN1 | NM_013255 |
| MKNK1 | NM_003684 |
| MKRN2 | NM_014160 |
| MLEC | NM_014730 |
| MLF2 | NM_005439 |
| MLH1 | NM_000249 |
| MLLT1 | NM_005934 |
| MLLT10 | NM_001195628 |
| MLST8 | NM_022372 |
| MLX | NM_170607 |
| MMAA | NM_172250 |
| MMADHC | NM_015702 |
| MMS19 | NM_022362 |
| MNAT1 | NM_002431 |
| MNF1 | NM_032340 |
| MOB4 | NM_015387 |
| MOGS | NM_006302 |
| MON1A | NM_032355 |
| MON2 | NM_015026 |
| MORC2 | NM_014941 |
| MORF4L2 | NM_012286 |
| MOSPD1 | NM_019556 |
| MPC2 | NM_015415 |
| MPDU1 | NM_004870 |
| MPG | NM_002434 |
| MPHOSPH10 | NM_005791 |
| MPI | NM_002435 |
| MPLKTP | NM_138701 |
| MPND | NM_032868 |
| MPPE1 | NM_023075 |
| MPV17L2 | NM_032683 |
| MRFAP1 | NM_033296 |
| MRFAP1L1 | NM_203462 |
| MRI1 | NM_032285 |
| MRN1 | NM_024864 |
| MRP63 | NM_024026 |
| MRPL1 | NM_020236 |
| MRPL10 | NM_145255 |
| MRPL11 | NM_016050 |
| MRPL12 | NM_002949 |
| MRPL13 | NM_014078 |
| MRPL14 | NM_032111 |
| MRPL15 | NM_014175 |
| MRPL16 | NM_017840 |
| MRPL17 | NM_022061 |
| MRPL18 | NM_014161 |
| MRPL19 | NM_014763 |
| MRPL2 | NM_015950 |
| MRPL20 | NM_017971 |
| MRPL21 | NM_181514 |
| MRPL22 | NM_014180 |
| MRPL23 | NM_021134 |
| MRPL24 | NM_024540 |
| MRPL27 | NM_016504 |
| MRPL28 | NM_006428 |
| MRPL3 | NM_007208 |
| MRPL30 | NM_145212 |
| MRPL32 | NM_031903 |
| MRPL33 | NM_004891 |
| MRPL35 | NM_016622 |
| MRPL36 | NM_032479 |
| MRPL37 | NM_016491 |
| MRPL38 | NM_032478 |
| MRPL4 | NM_015956 |
| MRPL40 | NM_003776 |
| MRPL41 | NM_032477 |
| MRPL42 | NM_014050 |
| MRPL43 | NM_032112 |
| MRPL44 | NM_022915 |
| MRPL45 | NM_032351 |
| MRPL46 | NM_022163 |
| MRPL47 | NM_020409 |
| MRPL48 | NM_016055 |
| MRPL49 | NM_004927 |
| MRPL50 | NM_019051 |
| MRPL51 | NM_016497 |
| MRPL52 | NM_178336 |
| MRPL53 | NM_053050 |
| MRPL54 | NM_172251 |
| MRPL55 | NM_181441 |
| MRPL9 | NM_031420 |
| MRPS10 | NM_018141 |
| MRPS11 | NM_022839 |
| MRPS12 | NM_021107 |
| MRPS14 | NM_022100 |
| MRPS15 | NM_031280 |
| MRPS16 | NM_016065 |
| MRPS17 | NM_015969 |
| MRPS18A | NM_018135 |
| MRPS18B | NM_014046 |
| MRPS18C | NM_016067 |
| MRPS2 | NM_016034 |
| MRPS21 | NM_018997 |
| MRPS22 | NM_020191 |
| MRPS23 | NM_016070 |
| MRPS24 | NM_032014 |
| MRPS25 | NM_022497 |
| MRPS26 | NM_030811 |
| MRPS27 | NM_015084 |
| MRPS28 | NM_014018 |
| MRPS30 | NM_016640 |
| MRPS31 | NM_005830 |
| MRPS33 | NM_016071 |
| MRPS34 | NM_023936 |
| MRPS35 | NM_021821 |
| MRPS5 | NM_031902 |
| MRPS6 | NM_032476 |
| MRPS7 | NM_015971 |
| MRPS9 | NM_182640 |
| MRRF | NM_138777 |
| MRS2 | NM_020662 |
| MRTO4 | NM_016183 |
| MSANTD3 | NM_080655 |
| MSH3 | NM_002439 |
| MSH6 | NM_000179 |
| MSL3 | NM_006800 |
| MSMP | NM_001044264 |
| MSRA | NM_012331 |
| MSRB2 | NM_012228 |
| MTA2 | NM_004739 |
| MTCH1 | NM_014341 |
| MTCH2 | NM_014342 |
| MTDH | NM_178812 |
| MTERFD1 | NM_015942 |
| MTERFD2 | NM_182501 |
| MTERFD3 | NM_025198 |
| MTEMT | NM_139242 |
| MTFR1 | NM_014637 |
| MTFR1L | NM_019557 |
| MTIF3 | NM_152912 |
| MTM1 | NM_000252 |
| MTMR1 | NM_003828 |
| MTMR3 | NM_021090 |
| MTMR6 | NM_004685 |
| MTO1 | NM_012123 |
| MTPAP | NM_018109 |
| MTRR | NM_002454 |
| MTSS1 | NM_014751 |
| MTX2 | NM_006554 |
| MUL1 | NM_024544 |
| MUS81 | NM_025128 |
| MUT | NM_000255 |
| MVD | NM_002461 |
| MXD4 | NM_006454 |
| MXI1 | NM_005962 |
| MYBBP1A | NM_014520 |
| MYEOV2 | NM_001163424 |
| MYL12B | NM_033546 |
| MYNN | NM_018657 |
| MYO1E | NM_004998 |
| MYPOP | NM_001012643 |
| MZF1 | NM_003422 |
| MZT2A | NM_001085365 |
| MZT2B | NM_025029 |
| N4BP1 | NM_153029 |
| N4BP2L2 | NM_014887 |
| NAA10 | NM_003491 |
| NAA15 | NM_057175 |
| NAA20 | NM_016100 |
| NAA38 | NM_016200 |
| NAA50 | NM_025146 |
| NAA60 | NM_024845 |
| NABP2 | NM_024068 |
| NACA | NM_001113202 |
| NACA2 | NM_199290 |
| NACC1 | NM_052876 |
| NACC2 | NM_144653 |
| NAE1 | NM_003905 |
| NAMPT | NM_005745 |
| NANS | NM_018946 |
| NAP1L4 | NM_005969 |
| NAPA | NM_003827 |
| NARF | NM_012336 |
| NARFL | NM_022493 |
| NARG2 | NM_024611 |
| NARS | NM_004539 |
| NARS2 | NM_024678 |
| NAT10 | NM_024662 |
| NBN | NM_002485 |
| NBR1 | NM_005899 |
| NCAPII2 | NM_014551 |
| NCBP2 | NM_007362 |
| NCK1 | NM_006153 |
| NCKIPSD | NM_016453 |
| NCL | NM_005381 |
| NCLN | NM_020170 |
| NCOA1 | NM_003743 |
| NCOA6 | NM_014071 |
| NCOR1 | NM_006311 |
| NCSTN | NM_015331 |
| NDEL1 | NM_030808 |
| NDFIP1 | NM_030571 |
| NDNL2 | NM_138704 |
| NDST1 | NM_001543 |
| NDUFA10 | NM_004544 |
| NDUFA11 | NM_175614 |
| NDUFA12 | NM_018838 |
| NDUFA13 | NM_015965 |
| NDUFA2 | NM_002488 |
| NDUFA3 | NM_004542 |
| NDUFA4 | NM_002489 |
| NDUFA5 | NM_005000 |
| NDUFA6 | NM_002490 |
| NDUFA7 | NM_005001 |
| NDUFA8 | NM_014222 |
| NDUFA9 | NM_005002 |
| NDUFAF2 | NM_174889 |
| NDUFAF3 | NM_199069 |
| NDUFAF4 | NM_014165 |
| NDUFB10 | NM_004548 |
| NDUFB11 | NM_019056 |
| NDUFB2 | NM_004546 |
| NDUFB3 | NM_002491 |
| NDUFB4 | NM_004547 |
| NDUFB5 | NM_002492 |
| NDUFB6 | NM_002493 |
| NDUFB7 | NM_004146 |
| NDUFB8 | NM_005004 |
| NDUFB9 | NM_005005 |
| NDUFC1 | NM_002494 |
| NDUFC2 | NM_004549 |
| NDUFC2-KCTD14 | NM_001203260 |
| NDUFS2 | NM_004550 |
| NDUFS3 | NM_004551 |
| NDUFS4 | NM_002495 |
| NDUFS5 | NM_004552 |
| NDUFS6 | NM_004553 |
| NDUFS7 | NM_024407 |
| NDUFS8 | NM_002496 |
| NDUFV1 | NM_007103 |
| NDUFV2 | NM_021074 |
| NECAP1 | NM_015509 |
| NEDD8 | NM_006156 |
| NEDD8-MDP1 | NM_001199823 |
| NEIL2 | NM_145043 |
| NEK4 | NM_003157 |
| NEK9 | NM_033116 |
| NELFB | NM_015456 |
| NELFCD | NM_198976 |
| NELFE | NM_002904 |
| NENF | NM_013349 |
| NEU1 | NM_000434 |
| NF2 | NM_000268 |
| NFATC2IP | NM_032815 |
| NFE2L2 | NM_006164 |
| NFIL3 | NM_005384 |
| NFKBIB | NM_002503 |
| NFKBIL1 | NM_005007 |
| NFU1 | NM_015700 |
| NFX1 | NM_002504 |
| NFYB | NM_006166 |
| NFYC | NM_014223 |
| NGDN | NM_015514 |
| NGLY1 | NM_018297 |
| NGRN | NM_001033088 |
| NHP2 | NM_017838 |
| NHP2L1 | NM_005008 |
| NIF3L1 | NM_021824 |
| NINJ1 | NM_004148 |
| NIP7 | NM_016101 |
| NIPA2 | NM_030922 |
| NIPBL | NM_015384 |
| NISCH | NM_001276293 |
| NIT1 | NM_005600 |
| NIT2 | NM_020202 |
| NKAP | NM_024528 |
| NKTRAS2 | NM_017595 |
| NMD3 | NM_015938 |
| NME1-NME2 | NM_001018136 |
| NME2 | NM_002512 |
| NME3 | NM_002513 |
| NME6 | NM_005793 |
| NMRK1 | NM_017881 |
| NMT1 | NM_021079 |
| NOA1 | NM_032313 |
| NOB1 | NM_014062 |
| NOC2L | NM_015658 |
| NOL10 | NM_024894 |
| NOL11 | NM_015462 |
| NOL12 | NM_024313 |
| NOL6 | NM_022917 |
| NOL7 | NM_019167 |
| NOL8 | NM_017948 |
| NNOLC1 | NM_004741 |
| NOM1 | NM_138400 |
| NON0 | NM_007363 |
| NOP10 | NM_018648 |
| NOP14 | NM_003703 |
| NOP16 | NM_016391 |
| NOP2 | NM_006170 |
| NOP56 | NM_006392 |
| NOP58 | NM_015934 |
| NOP9 | NM_174913 |
| NPC1 | NM_000271 |
| NPC2 | NM_006432 |
| NPLOC4 | NM_017921 |
| NPRL2 | NM_006545 |
| NPRL3 | NM_001039476 |
| NQO2 | NM_000904 |
| NR1H2 | NM_007121 |
| NR2C1 | NM_003297 |
| NR2C2AP | NM_176880 |
| NR3C2 | NM_001166104 |
| NRBP1 | NM_013392 |
| NRDE2 | NM_017970 |
| NRIP1 | NM_003489 |
| NSA2 | NM_014886 |
| NSD1 | NM_022455 |
| NSDHL | NM_015922 |
| NSFL10 | NM_016143 |
| NSMCE1 | NM_145080 |
| NSMCE2 | NM_173685 |
| NSMCE4A | NM_017615 |
| NSRP1 | NM_032141 |
| NSUN2 | NM_017755 |
| NSUN5 | NM_018044 |
| NSUN6 | NM_182543 |
| NT5C | NM_014595 |
| NT5C3 | NM_016489 |
| NT5DC1 | NM_152729 |
| NTAN1 | NM_173474 |
| NTMT1 | NM_014064 |
| NTPCR | NM_032324 |
| NUB1 | NM_016118 |
| NUBP1 | NM_002484 |
| NUBP2 | NM_012225 |
| NUCB1 | NM_006184 |
| NUCKS1 | NM_022731 |
| NUDC | NM_006600 |
| NUDCD1 | NM_032869 |
| NUDCD2 | NM_145266 |
| NUDT14 | NM_177533 |
| NUDT15 | NM_018283 |
| NUDT2 | NM_001161 |
| NUDT21 | NM_007006 |
| NUDT28 | NM_032344 |
| NUDT3 | NM_006703 |
| NUDT9 | NM_024047 |
| NUFIP2 | NM_020772 |
| NUP107 | NM_020401 |
| NUP133 | NM_018230 |
| NUP153 | NM_005124 |
| NUP54 | NM_017426 |
| NUP62 | NM_012346 |
| NUP85 | NM_024844 |
| NUPL2 | NM_007342 |
| NUTF2 | NM_005796 |
| NXF1 | NM_006362 |
| NXT1 | NM_013248 |
| OAT | NM_000274 |
| OAZ1 | NM_004152 |
| OAZ2 | NM_002537 |
| OBFC1 | NM_024928 |
| OCEL1 | NM_024578 |
| OCIAD1 | NM_017830 |
| ODC1 | NM_002539 |
| OGFOD1 | NM_018233 |
| OGFOD3 | NM_024648 |
| OGFR | NM_007346 |
| OGG1 | NM_016827 |
| OGT | NM_181672 |
| OLA1 | NM_013341 |
| OPA1 | NM_015560 |
| OPA3 | NM_025136 |
| ORC4 | NM_002552 |
| ORMDL1 | NM_016467 |
| ORMDL2 | NM_014182 |
| ORMDL3 | NM_139280 |
| OS9 | NM_006812 |
| OSBP | NM_002556 |
| OSBPL2 | NM_014835 |
| OSBPL9 | NM_024586 |
| OSGEP | NM_017807 |
| OSGIN2 | NM_004337 |
| OSTM1 | NM_014028 |
| OTUB1 | NM_017670 |
| OTUD5 | NM_017602 |
| OVCA2 | NM_080822 |
| OXA1L | NM_005015 |
| OXNAD1 | NM_138381 |
| P4HTM | NM_177938 |
| PA2G4 | NM_006191 |
| PABPN1 | NM_004543 |
| PACSIN2 | NM_007229 |
| PAF1 | NM_019088 |
| PAFAH1B1 | NM_000430 |
| PAGR1 | NM_024516 |
| PAICS | NM_006452 |
| PAIP1 | NM_006451 |
| PAIP2 | NM_016480 |
| PAK1IP1 | NM_017906 |
| PAK2 | NM_002577 |
| PAM16 | NM_016069 |
| PANK2 | NM_024960 |
| PANK3 | NM_024594 |
| PANK4 | NM_018216 |
| PANX1 | NM_015368 |
| PAPD4 | NM_173797 |
| PAPD7 | NM_006999 |
| PAPOLA | NM_032632 |
| PARK7 | NM_007262 |
| PARL | NM_018622 |
| PARN | NM_002582 |
| PARP1 | NM_001618 |
| PARP3 | NM_005485 |
| PARP9 | NM_001146106 |
| PATL1 | NM_152716 |
| PATZ1 | NM_014323 |
| PAXBP1 | NM_013329 |
| PBDC1 | NM_016500 |
| PBX2 | NM_002586 |
| PCBP1 | NM_006196 |
| PCBP2 | NM_005016 |
| PCDHGB5 | NM_032099 |
| PCF11 | NM_015885 |
| PCGF1 | NM_032673 |
| PCGF5 | NM_032373 |
| PC1D2 | NM_018386 |
| PC1F1 | NM_022104 |
| PCM1 | NM_006197 |
| PCMT1 | NM_005389 |
| PCNA | NM_002592 |
| PCNX | NM_014982 |
| PCNXL4 | NM_022495 |
| PCSK7 | NM_004716 |
| PCYOX1 | NM_016297 |
| PCYT1A | NM_005017 |
| PDAP1 | NM_014891 |
| PDCD2 | NM_002598 |
| PDCD5 | NM_004708 |
| PDCD6 | NM_013232 |
| PDCD6IP | NM_013374 |
| PDE12 | NM_177966 |
| PDE6D | NM_002601 |
| PDGFC | NM_016205 |
| PDHB | NM_000925 |
| PDHX | NM_003477 |
| PDK2 | NM_001199900 |
| PDLIM5 | NM_001256425 |
| PDP2 | NM_020786 |
| PDS5A | NM_001100399 |
| PDZD11 | NM_016484 |
| PDZDS | NM_173791 |
| PEBP1 | NM_002567 |
| PEF1 | NM_012392 |
| PELO | NM_015946 |
| PELP1 | NM_014389 |
| PEPD | NM_000285 |
| PES1 | NM_014303 |
| PET100 | NM_001171155 |
| PET117 | NM_001164811 |
| PEX1 | NM_000466 |
| PEX11A | NM_001271573 |
| PEX11B | NM_003846 |
| PEX12 | NM_000286 |
| PEX13 | NM_002618 |
| PEX14 | NM_004565 |
| PEX15 | NM_004813 |
| PEX19 | NM_002857 |
| PEX2 | NM_000318 |
| PEX25 | NM_017929 |
| PEX5 | NM_000319 |
| PEX6 | NM_000287 |
| PFDN2 | NM_012394 |
| PFDN4 | NM_002623 |
| PFDN5 | NM_002624 |
| PFDN6 | NM_014260 |
| PFN1 | NM_005022 |
| PGAM5 | NM_138575 |
| PGBD3 | NM_170753 |
| PCK1 | NM_000291 |
| PGLS | NM_012088 |
| PGP | NM_001042371 |
| PGPEP1 | NM_017712 |
| PGRMC2 | NM_006320 |
| PHACTR4 | NM_023923 |
| PHAX | NM_032177 |
| PHB | NM_002634 |
| PHB2 | NM_001144831 |
| PHC2 | NM_004427 |
| PHF10 | NM_018288 |
| PHF12 | NM_020889 |
| PHF20L1 | NM_016018 |
| PHF23 | NM_024297 |
| PHF5A | NM_032758 |
| PHKB | NM_000293 |
| PHPT1 | NM_014172 |
| PHRF1 | NM_020901 |
| PI4K2A | NM_018425 |
| PI4KA | NM_002650 |
| PI4KB | NM_002651 |
| PIAS1 | NM_016166 |
| PICALM | NM_007166 |
| PICK1 | NM_012407 |
| PIGC | NM_002642 |
| PIGF | NM_002643 |
| PIGG | NM_017733 |
| PIGH | NM_004569 |
| PIGK | NM_005482 |
| PIGP | NM_153681 |
| PIGS | NM_033198 |
| PIGT | NM_015937 |
| PIGU | NM_080476 |
| PIGW | NM_178517 |
| PIGX | NM_017861 |
| PIGY | NM_001042616 |
| PIH1D1 | NM_017916 |
| PIK3C3 | NM_002647 |
| PIK3CB | NM_006219 |
| PIK3R1 | NM_181504 |
| PIK3R4 | NM_014602 |
| PIN1 | NM_006221 |
| PINK1 | NM_032409 |
| PINX1 | NM_017884 |
| PIP5K1A | NM_003557 |
| PITHD1 | NM_020362 |
| PITPNA | NM_006224 |
| PITPNB | NM_012399 |
| PITRM1 | NM_014889 |
| PLA2G12A | NM_030821 |
| PLAA | NM_001031689 |
| PLBD2 | NM_173542 |
| PLD3 | NM_012268 |
| PLEKHA1 | NM_021622 |
| PLEKHJ1 | NM_018049 |
| PLEKHM1 | NM_014798 |
| PLGRKT | NM_018465 |
| PLIN3 | NM_005817 |
| PLOD1 | NM_000302 |
| PLOD3 | NM_001084 |
| PLRG1 | NM_002669 |
| PMF1 | NM_007221 |
| PMF1-BGLAP | NM_001199661 |
| PMPCA | NM_015160 |
| PMPCB | NM_004279 |
| PMS1 | NM_001128144 |
| PMVK | NM_006556 |
| PNISR | NM_015491 |
| PNKD | NM_001077399 |
| PNKP | NM_007254 |
| PNN | NM_002687 |
| PNO1 | NM_020143 |
| PNPLA6 | NM_006702 |
| PNPLA8 | NM_015723 |
| PNPQ | NM_018129 |
| PNPT1 | NM_033109 |
| PNRC2 | NM_017761 |
| POFUT1 | NM_015352 |
| POLD2 | NM_006230 |
| POLDIP2 | NM_015584 |
| POLDIP3 | NM_032311 |
| POLE3 | NM_017443 |
| POLE4 | NM_019896 |
| POLG | NM_002693 |
| POLH | NM_006502 |
| POLK | NM_016218 |
| POLL | NM_013274 |
| POLM | NM_013284 |
| POLR1C | NM_203290 |
| POLR1D | NM_015972 |
| POLR1E | NM_022490 |
| POLR2A | NM_000937 |
| POLR2B | NM_000938 |
| POLR2C | NM_032940 |
| POLR2D | NM_004805 |
| POLR2E | NM_002695 |
| POLR2F | NM_021974 |
| POLR2G | NM_002696 |
| POLR2H | NM_006232 |
| POLR2I | NM_006233 |
| POLR2J | NM_006234 |
| POLR2K | NM_005034 |
| POLR2L | NM_021128 |
| POLR3C | NM_006468 |
| POLR3E | NM_018119 |
| POLR3GL | NM_032305 |
| POLR3K | NM_016310 |
| POM121 | NM_172020 |
| POM121C | NM_001099413 |
| POMGNT1 | NM_017739 |
| POMP | NM_015932 |
| POMT1 | NM_007171 |
| POP4 | NM_006627 |
| POP5 | NM_015918 |
| POP7 | NM_005837 |
| PPA1 | NM_021129 |
| PPA2 | NM_176867 |
| PPAN | NM_020230 |
| PPAN-P2RY11 | NM_001040664 |
| PPARA | NM_005036 |
| PPARD | NM_006238 |
| PPCS | NM_024664 |
| PPFIA1 | NM_003626 |
| PPHLN1 | NM_016488 |
| PPID | NM_005038 |
| PPIE | NM_006112 |
| PPIF | NM_005729 |
| PPIG | NM_004792 |
| PPIH | NM_006347 |
| PPIIA | NM_139126 |
| PPM1A | NM_021003 |
| PPM1B | NM_002706 |
| PPP1CA | NM_002708 |
| PPP1CC | NM_002710 |
| PPP1R10 | NM_002714 |
| PPP1R11 | NM_021959 |
| PPP1R15B | NM_032833 |
| PPP1R37 | NM_019121 |
| PPP1R7 | NM_002712 |
| PPP1R8 | NM_002713 |
| PPP2CA | NM_002715 |
| PPP2CB | NM_001009552 |
| PPP2R1A | NM_014225 |
| PPP2R2A | NM_002717 |
| PPP2R2D | NM_018461 |
| PPP2R3C | NM_017917 |
| PPP2R4 | NM_021131 |
| PPP2R5A | NM_006243 |
| PPP2R5B | NM_006244 |
| PPP2R5C | NM_002719 |
| PPP2R5D | NM_006245 |
| PPP2R5E | NM_006246 |
| PPP4C | NM_002720 |
| PPP4R1 | NM_005134 |
| PPP4R2 | NM_174907 |
| PPP5C | NM_006247 |
| PPP6C | NM_002721 |
| PPP6R2 | NM_014678 |
| PPP6R3 | NM_018312 |
| PPWD1 | NM_015342 |
| PQBP1 | NM_005710 |
| PQLC1 | NM_025078 |
| PQLC2 | NM_017765 |
| PRADC1 | NM_032319 |
| PRCC | NM_005973 |
| PRDM4 | NM_012406 |
| PRDX1 | NM_002574 |
| PRDX2 | NM_005809 |
| PRDX3 | NM_006793 |
| PRDX5 | NM_012094 |
| PRDX6 | NM_004905 |
| PREB | NM_013388 |
| PREP | NM_002725 |
| PRKAA1 | NM_006251 |
| PRKAB1 | NM_006253 |
| PRKACA | NM_002730 |
| PRKAG1 | NM_002733 |
| PRKAR1A | NM_002734 |
| PRKRIP1 | NM_024653 |
| PRMT1 | NM_198318 |
| PRMT5 | NM_006109 |
| PRMT7 | NM_019023 |
| PROSC | NM_007198 |
| PRPF18 | NM_003675 |
| PRPF19 | NM_014502 |
| PRPF3 | NM_004698 |
| PRPF31 | NM_015629 |
| PRPF4 | NM_004697 |
| PRPF40A | NM_017892 |
| PRPF4B | NM_003913 |
| PRPF6 | NM_012469 |
| PRPF8 | NM_006445 |
| PRPS1 | NM_002764 |
| PRPSAP1 | NM_002766 |
| PRR14 | NM_024031 |
| PRRC1 | NM_130809 |
| PRRC2A | NM_004638 |
| PRRC2B | NM_013318 |
| PRUNE | NM_021222 |
| PSEN1 | NM_000021 |
| PSEN2 | NM_000447 |
| PSENEN | NM_172341 |
| PSKH1 | NM_006742 |
| PSMA1 | NM_002786 |
| PSMA2 | NM_002787 |
| PSMA3 | NM_002788 |
| PSMA4 | NM_002789 |
| PSMA5 | NM_002790 |
| PSMA6 | NM_002791 |
| PSMA7 | NM_002792 |
| PSMB1 | NM_002793 |
| PSMB2 | NM_002794 |
| PSMB3 | NM_002795 |
| PSMB4 | NM_002796 |
| PSMB5 | NM_002797 |
| PSMB6 | NM_002798 |
| PSMB7 | NM_002799 |
| PSMC2 | NM_002803 |
| PSMC3 | NM_002804 |
| PSMC4 | NM_006503 |
| PSMC5 | NM_002805 |
| PSMC6 | NM_002806 |
| PSMD1 | NM_002807 |
| PSMD10 | NM_002814 |
| PSMD11 | NM_002815 |
| PSMD12 | NM_002816 |
| PSMD13 | NM_002817 |
| PSMD14 | NM_005805 |
| PSMD2 | NM_002808 |
| PSMD3 | NM_002809 |
| PSMD4 | NM_002810 |
| PSMD5 | NM_005047 |
| PSMD6 | NM_014814 |
| PSMD7 | NM_002811 |
| PSMD8 | NM_002814 |
| PSMD9 | NM_002813 |
| PSME1 | NM_006263 |
| PSME3 | NM_005789 |
| PSMF1 | NM_006814 |
| PSMG2 | NM_020232 |
| PSMG3 | NM_032302 |
| PSMG4 | NM_001128591 |
| PSPC1 | NM_001042414 |
| PTCD1 | NM_015545 |
| PTCD3 | NM_017952 |
| PTDSS1 | NM_014754 |
| PTEN | NM_000314 |
| PTGES2 | NM_025072 |
| PTGES3 | NM_006601 |
| PTOV1 | NM_017432 |
| PTP4A2 | NM_080391 |
| PTPMT1 | NM_175732 |
| PTPN1 | NM_002827 |
| PTPN11 | NM_002834 |
| PTPN23 | NM_015466 |
| PTRH1 | NM_001002913 |
| PTRH2 | NM_016077 |
| PTRHD1 | NM_001013663 |
| PUF60 | NM_014281 |
| PUM1 | NM_014676 |
| PUM2 | NM_015317 |
| PURA | NM_005859 |
| PURB | NM_033224 |
| PUS3 | NM_031307 |
| PUS7 | NM_019042 |
| PUSL1 | NM_153339 |
| PWP1 | NM_007062 |
| PWP2 | NM_005049 |
| PWWP2A | NM_052927 |
| PXMP4 | NM_007238 |
| PYCR2 | NM_013328 |
| PYGO2 | NM_138300 |
| PYURF | NM_032906 |
| QARS | NM_005051 |
| QRICH1 | NM_017730 |
| QRSL1 | NM_018292 |
| QSOX1 | NM_002826 |
| QTRT1 | NM_031209 |
| R3HCC1 | NM_001136108 |
| R3HDM2 | NM_014925 |
| RAB10 | NM_016131 |
| RAB11A | NM_004663 |
| RAB11B | NM_004218 |
| RAB14 | NM_016322 |
| RAB18 | NM_021252 |
| RAB1A | NM_004161 |
| RAB1B | NM_030981 |
| RAB21 | NM_014999 |
| RAB22A | NM_020673 |
| RAB2A | NM_002865 |
| RAB2B | NM_001163380 |
| RAB3GAP1 | NM_012233 |
| RAB3GAP2 | NM_012414 |
| RAB40C | NM_021168 |
| RAB4A | NM_004578 |
| RAB5A | NM_004162 |
| RAB5B | NM_002868 |
| RAB5C | NM_004583 |
| RAB6A | NM_002869 |
| RAB7A | NM_004637 |
| RAB9A | NM_004251 |
| RABEP1 | NM_004703 |
| RABEPK | NM_005833 |
| RABGEF1 | NM_014504 |
| RABGGTA | NM_004581 |
| RABGGTB | NM_004582 |
| RAD1 | NM_002853 |
| RAD17 | NM_002873 |
| RAD23B | NM_002874 |
| RAD50 | NM_005732 |
| RAD51C | NM_002876 |
| RAF1 | NM_002880 |
| RALA | NM_005402 |
| RALBP1 | NM_006788 |
| RALY | NM_007367 |
| RAN | NM_006325 |
| RANBP1 | NM_002882 |
| RANBP2 | NM_006267 |
| RANBP3 | NM_003624 |
| RANBP6 | NM_012416 |
| RANGAP1 | NM_002883 |
| RANGRF | NM_016492 |
| RAP1A | NM_002884 |
| RAPGEF1 | NM_005312 |
| RAPGEF2 | NM_014247 |
| RARS | NM_002887 |
| RARS2 | NM_020320 |
| RB1CC1 | NM_014781 |
| RBAK | NM_021163 |
| RBBP4 | NM_005610 |
| RBBP7 | NM_002893 |
| RBCK1 | NM_006462 |
| RBFA | NM_024805 |
| RBM10 | NM_005676 |
| RBM12 | NM_006047 |
| RBM12B | NM_203390 |
| RBM14 | NM_006328 |
| RBM14-RBM4 | NM_001198845 |
| RBM15 | NM_022768 |
| RBM15B | NM_013286 |
| RBM17 | NM_032905 |
| RBM18 | NM_033117 |
| RBM19 | NM_016196 |
| RBM23 | NM_018107 |
| RBM27 | NM_018989 |
| RBM28 | NM_018077 |
| RBM33 | NM_053043 |
| RBM34 | NM_015014 |
| RBM39 | NM_004902 |
| RBM4 | NM_002806 |
| RBM41 | NM_018301 |
| RBM42 | NM_024321 |
| RBM5 | NM_005778 |
| RBM6 | NM_005777 |
| RBM7 | NM_016090 |
| RBM8A | NM_005105 |
| RBMX | NM_002139 |
| RBMXL1 | NM_019610 |
| RBX1 | NM_014248 |
| RC3H2 | NM_018835 |
| RCAN1 | NM_004414 |
| RCHY1 | NM_015436 |
| RCN2 | NM_002902 |
| RDH14 | NM_020905 |
| RDX | NM_002906 |
| REEP3 | NM_001001330 |
| REEP5 | NM_005669 |
| RELA | NM_021975 |
| REPIN1 | NM_013400 |
| REPS1 | NM_031922 |
| RER1 | NM_007033 |
| REST | NM_005612 |
| REXO1 | NM_020695 |
| RFC1 | NM_002913 |
| RFC2 | NM_002914 |
| RFC5 | NM_007370 |
| RFK | NM_018339 |
| RFNG | NM_002917 |
| RFT1 | NM_052859 |
| RFWD2 | NM_022457 |
| RFXANK | NM_003721 |
| RGP1 | NM_001080496 |
| RHBDD1 | NM_032276 |
| RHBDD3 | NM_012265 |
| RHOA | NM_001664 |
| RHOB | NM_004040 |
| RHOT1 | NM_018307 |
| RHOT2 | NM_138769 |
| RIC8A | NM_021932 |
| RIN2 | NM_018993 |
| RING1 | NM_002931 |
| RINT1 | NM_021930 |
| RIOK1 | NM_031480 |
| RIOK2 | NM_018343 |
| RIOK3 | NM_003831 |
| RIPK1 | NM_003804 |
| RMDN1 | NM_016033 |
| RMDN3 | NM_018145 |
| RMI1 | NM_024945 |
| RMND1 | NM_017909 |
| RMND5A | NM_022780 |
| RMND5B | NM_022762 |
| RNASEH1 | NM_002986 |
| RNASEH2C | NM_032193 |
| RNASEK | NM_001004333 |
| RNF10 | NM_014868 |
| RNF103 | NM_005667 |
| RNF11 | NM_014372 |
| RNF111 | NM_017610 |
| RNF113A | NM_006978 |
| RNF115 | NM_014455 |
| RNF121 | NM_018320 |
| RNF126 | NM_194460 |
| RNF13 | NM_007282 |
| RNF14 | NM_004290 |
| RNF141 | NM_016422 |
| RNF146 | NM_030963 |
| RNF167 | NM_015528 |
| RNF181 | NM_016494 |
| RNF185 | NM_152267 |
| RNF187 | NM_001010858 |
| RNF216 | NM_207111 |
| RNF220 | NM_018150 |
| RNF25 | NM_022453 |
| RNF26 | NM_032015 |
| RNF31 | NM_017999 |
| RNF34 | NM_025126 |
| RNF4 | NM_002938 |
| RNF40 | NM_014771 |
| RNF5 | NM_006913 |
| RNF6 | NM_005977 |
| RNF7 | NM_014245 |
| RNH1 | NM_002939 |
| RNMTL1 | NM_018146 |
| RNPEP | NM_020216 |
| ROMO1 | NM_080748 |
| RP9 | NM_203288 |
| RPA2 | NM_002946 |
| RPA3 | NM_002947 |
| RPAIN | NM_001033002 |
| RPAP3 | NM_024604 |
| RPF1 | NM_025065 |
| RPF2 | NM_032194 |
| RPL10A | NM_007104 |
| RPL11 | NM_000975 |
| RPL14 | NM_003973 |
| RPL26L1 | NM_016093 |
| RPL27 | NM_000988 |
| RPL30 | NM_000989 |
| RPL31 | NM_000993 |
| RPL32 | NM_000994 |
| RPL34 | NM_000995 |
| RPL35 | NM_007209 |
| RPL35A | NM_000996 |
| RPL36AL | NM_001001 |
| RPL4 | NM_000968 |
| RPL6 | NM_000970 |
| RPL7L1 | NM_198486 |
| RPL8 | NM_000973 |
| RPN1 | NM_002950 |
| RPN2 | NM_002951 |
| RPP14 | NM_007042 |
| RPP25L | NM_148178 |
| RPP30 | NM_006413 |
| RPP38 | NM_006414 |
| RPRD1B | NM_021215 |
| RPS13 | NM_001017 |
| RPS19BP1 | NM_194326 |
| RPS23 | NM_001025 |
| RPS24 | NM_001026 |
| RPS27L | NM_015920 |
| RPS5 | NM_001009 |
| RPS6 | NM_001010 |
| RPS6KA3 | NM_004586 |
| RPS6KB1 | NM_003161 |
| RPS6KB2 | NM_003952 |
| RPUSD3 | NM_173659 |
| RQCD1 | NM_005444 |
| RRAGA | NM_006570 |
| RRM1 | NM_001033 |
| RRN3 | NM_018427 |
| RRNAD1 | NM_015997 |
| RRP1 | NM_003683 |
| RRP36 | NM_033112 |
| RRP7A | NM_015703 |
| RRP8 | NM_015324 |
| RRS1 | NM_015169 |
| RSAD1 | NM_018346 |
| RSBN1L | NM_198467 |
| RSC1A1 | NM_006511 |
| RSL1D1 | NM_015659 |
| RSPRY1 | NM_133368 |
| RSRC1 | NM_016625 |
| RSRC2 | NM_023012 |
| RTCA | NM_003729 |
| RTFDC1 | NM_016407 |
| RTN4 | NM_007008 |
| RUFY1 | NM_025158 |
| RUVBL1 | NM_003707 |
| RWDD1 | NM_015952 |
| RWDD3 | NM_015485 |
| RXRA | NM_002957 |
| RXRB | NM_021976 |
| SAE1 | NM_005500 |
| SAMD1 | NM_138352 |
| SAMD4B | NM_018028 |
| SAMD8 | NM_144660 |
| SAMM50 | NM_015380 |
| SAPI8 | NM_005870 |
| SAP30 | NM_003864 |
| SAP30BP | NM_013260 |
| SAP30L | NM_024632 |
| SAR1A | NM_020150 |
| SARNP | NM_033082 |
| SARS | NM_006513 |
| SART1 | NM_005146 |
| SART3 | NM_014706 |
| SAT2 | NM_133491 |
| SAV1 | NM_021818 |
| SBDS | NM_016038 |
| SCAF1 | NM_021228 |
| SCAF11 | NM_004719 |
| SCAF4 | NM_020706 |
| SCAF8 | NM_014892 |
| SCAMP2 | NM_005697 |
| SCAMP3 | NM_005698 |
| SCAND1 | NM_016558 |
| SCAP | NM_012235 |
| SCARB2 | NM_005506 |
| SCFD1 | NM_016106 |
| SCFD2 | NM_152540 |
| SCNM1 | NM_024041 |
| SCO1 | NM_004589 |
| SCO2 | NM_005138 |
| SCOC | NM_032547 |
| SCP2 | NM_001007250 |
| SCRIB | NM_015356 |
| SCRN3 | NM_024583 |
| SCYL1 | NM_020680 |
| SCYL2 | NM_017988 |
| SCYL3 | NM_020423 |
| SDAD1 | NM_018115 |
| SDCBP | NM_001007068 |
| SDCCAG3 | NM_006643 |
| SDCCAG8 | NM_006642 |
| SDE2 | NM_152608 |
| SDF2 | NM_006923 |
| SDF4 | NM_016176 |
| SDHA | NM_004168 |
| SDHAF2 | NM_017841 |
| SDHB | NM_003000 |
| SDHC | NM_003001 |
| SDHD | NM_003002 |
| SDR39U1 | NM_020195 |
| SEC11A | NM_014300 |
| SEC13 | NM_183352 |
| SEC16A | NM_014866 |
| SEC22B | NM_004892 |
| SEC22C | NM_004206 |
| SEC23A | NM_006364 |
| SEC23IP | NM_007190 |
| SEC24A | NM_021982 |
| SEC24B | NM_006323 |
| SEC24C | NM_004922 |
| SEC31A | NM_014933 |
| SEC61A1 | NM_013336 |
| SEC61B | NM_006808 |
| SEC61G | NM_014302 |
| SEC62 | NM_003262 |
| SEC63 | NM_007214 |
| SECISBP2 | NM_024077 |
| SEH1L | NM_031216 |
| SEL1L | NM_005065 |
| SELK | NM_021237 |
| SELO | NM_031454 |
| SELRC1 | NM_023077 |
| SELT | NM_016275 |
| SENP2 | NM_021627 |
| SENP3 | NM_015670 |
| SENP5 | NM_152699 |
| SENP6 | NM_015571 |
| SEPHS1 | NM_012247 |
| SERBP1 | NM_015640 |
| SERF2 | NM_001018108 |
| SERGEF | NM_012139 |
| SERINC1 | NM_020755 |
| SERINC3 | NM_006811 |
| SERPINB6 | NM_004568 |
| SERTAD2 | NM_014755 |
| SET | NM_003011 |
| SETD2 | NM_014159 |
| SETD3 | NM_032233 |
| SETD5 | NM_001080517 |
| SETD6 | NM_001160305 |
| SETD7 | NM_030648 |
| SETD8 | NM_020382 |
| SETDB1 | NM_012432 |
| SF1 | NM_004630 |
| SF3A1 | NM_005877 |
| SF3A3 | NM_006802 |
| SF3B1 | NM_012433 |
| SF3B14 | NM_016047 |
| SF3B2 | NM_006842 |
| SF3B3 | NM_012426 |
| SF3B4 | NM_005850 |
| SF3B5 | NM_031287 |
| SFSWAP | NM_004592 |
| SGK196 | NM_032237 |
| SGMS1 | NM_147156 |
| SGPL1 | NM_003901 |
| SGSM3 | NM_015705 |
| SGTA | NM_003021 |
| SH3BP5L | NM_030645 |
| SH3GLB1 | NM_016009 |
| SHARPIN | NM_030974 |
| SHOC2 | NM_007373 |
| SIAH1 | NM_003031 |
| SIAH2 | NM_005067 |
| SIGMAR1 | NM_005866 |
| SIKE1 | NM_025073 |
| SIL1 | NM_022464 |
| SIRT2 | NM_012237 |
| SIRT3 | NM_012239 |
| SIRT5 | NM_012241 |
| SIRT6 | NM_001193285 |
| SIVA1 | NM_006427 |
| SKIL | NM_005414 |
| SKIV2L | NM_006929 |
| SKIV2L2 | NM_015360 |
| SKP1 | NM_006930 |
| SLC15A4 | NM_145648 |
| SLC20A1 | NM_005415 |
| SLC25A11 | NM_003562 |
| SLC25A26 | NM_173471 |
| SLC25A28 | NM_031212 |
| SLC25A3 | NM_002635 |
| SLC25A32 | NM_030780 |
| SLC25A38 | NM_017875 |
| SLC25A39 | NM_016016 |
| SLC25A44 | NM_014655 |
| SLC25A46 | NM_138773 |
| SLC25A5 | NM_001152 |
| SLC27A4 | NM_005094 |
| SLC30A1 | NM_021194 |
| SLC30A5 | NM_022902 |
| SLC30A9 | NM_006345 |
| SLC35A2 | NM_005660 |
| SLC35A4 | NM_080670 |
| SLC35B1 | NM_005827 |
| SLC35B2 | NM_178148 |
| SLC35C2 | NM_015945 |
| SLC35E1 | NM_024881 |
| SLC35E3 | NM_018656 |
| SLC35F5 | NM_025181 |
| SLC38A2 | NM_018976 |
| SLC39A1 | NM_014437 |
| SLC39A3 | NM_144564 |
| SLC39A7 | NM_006979 |
| SLC41A3 | NM_017836 |
| SLC46A3 | NM_181785 |
| SLC48A1 | NM_017842 |
| SLIRP | NM_031210 |
| SLMO2 | NM_016045 |
| SLTM | NM_024755 |
| SMAD2 | NM_005901 |
| SMAD4 | NM_005359 |
| SMAD5 | NM_005903 |
| SMAP1 | NM_021940 |
| SMARCA2 | NM_003070 |
| SMARCA4 | NM_003072 |
| SMARCAL1 | NM_014140 |
| SMARCB1 | NM_003073 |
| SMARCE1 | NM_003079 |
| SMC1A | NM_006306 |
| SMC5 | NM_015110 |
| SMCR7L | NM_019008 |
| SMEK1 | NM_032560 |
| SMEK2 | NM_020463 |
| SMG5 | NM_015327 |
| SMG7 | NM_173156 |
| SMG8 | NM_018149 |
| SMIM11 | NM_058182 |
| SMIM12 | NM_138428 |
| SMIMS | NM_020425 |
| SMNDC1 | NM_005871 |
| SMPD1 | NM_000543 |
| SMPD4 | NM_017751 |
| SMU1 | NM_018225 |
| SMUG1 | NM_014311 |
| SNAP23 | NM_003825 |
| SNAP29 | NM_004782 |
| SNAP47 | NM_053052 |
| SNAPC3 | NM_001039697 |
| SNAPC5 | NM_006049 |
| SNAPIN | NM_012437 |
| SND1 | NM_014390 |
| SNF8 | NM_007241 |
| SNRNP200 | NM_014014 |
| SNRNP25 | NM_024571 |
| SNRNP27 | NM_006857 |
| SNRNP35 | NM_022717 |
| SNRNP40 | NM_004814 |
| SNRNP48 | NM_152551 |
| SNRNP70 | NM_003089 |
| SNRPA | NM_004596 |
| SNRPB | NM_003091 |
| SNRPB2 | NM_003092 |
| SNRPC | NM_003093 |
| SNRPD1 | NM_006938 |
| SNRPD2 | NM_004597 |
| SNRPD3 | NM_004175 |
| SNRPG | NM_003096 |
| SNUPN | NM_005701 |
| SNW1 | NM_012245 |
| SNX12 | NM_013346 |
| SNX13 | NM_015132 |
| SNX17 | NM_014748 |
| SNX18 | NM_052870 |
| SNX19 | NM_014758 |
| SNX2 | NM_003100 |
| SNX25 | NM_031953 |
| SNX3 | NM_003795 |
| SNX4 | NM_003794 |
| SNX5 | NM_014426 |
| SNX6 | NM_021249 |
| SNX9 | NM_016224 |
| SOCS4 | NM_080867 |
| SOCS6 | NM_004232 |
| SOD1 | NM_000454 |
| SON | NM_032195 |
| SPAG7 | NM_004890 |
| SPAG9 | NM_003971 |
| SPATA2 | NM_006038 |
| SPATA5L1 | NM_024063 |
| SPCS1 | NM_014041 |
| SPCS3 | NM_021928 |
| SPECC1L | NM_015330 |
| SPEN | NM_015001 |
| SPG11 | NM_025137 |
| SPG21 | NM_016630 |
| SPG7 | NM_003119 |
| SPHAR | NM_006542 |
| SPNS1 | NM_032038 |
| SPOP | NM_003563 |
| SPPL2B | NM_152988 |
| SPPL3 | NM_139015 |
| SPRYD3 | NM_032840 |
| SPRYD7 | NM_020456 |
| SPSB3 | NM_080861 |
| SPTSSA | NM_138288 |
| SPTY2D1 | NM_194285 |
| SRA1 | NM_001035235 |
| SRD5A3 | NM_024592 |
| SREBF2 | NM_004599 |
| SREK1IP1 | NM_173829 |
| SRM | NM_003132 |
| SRP14 | NM_003134 |
| SRP19 | NM_003135 |
| SRP34 | NM_003136 |
| SRP68 | NM_014230 |
| SRP72 | NM_006947 |
| SRP9 | NM_003133 |
| SRPR | NM_003139 |
| SRPRB | NM_021203 |
| SRR | NM_021947 |
| SRRD | NM_001013694 |
| SRRM1 | NM_005839 |
| SRSF1 | NM_006924 |
| SRSF10 | NM_006625 |
| SRSF11 | NM_004768 |
| SRSF2 | NM_003016 |
| SRSF3 | NM_003017 |
| SRSF4 | NM_005626 |
| SRSF7 | NM_001031684 |
| SRSF8 | NM_032102 |
| SS18L2 | NM_016305 |
| SSB | NM_003142 |
| SSBP1 | NM_003143 |
| SSNA1 | NM_003731 |
| SSR1 | NM_003144 |
| SSR2 | NM_003145 |
| SSR3 | NM_007107 |
| SSRP1 | NM_003146 |
| SSSCA1 | NM_006396 |
| SSU72 | NM_014188 |
| ST3GAL2 | NM_006927 |
| ST6GALNAC6 | NM_013443 |
| ST7 | NM_018412 |
| STAM | NM_003473 |
| STAM2 | NM_005843 |
| STAMBP | NM_006463 |
| STARD3 | NM_006804 |
| STARD7 | NM_020151 |
| STAT3 | NM_003150 |
| STAU1 | NM_004602 |
| STAU2 | NM_014393 |
| STIM1 | NM_003156 |
| STIP1 | NM_006819 |
| STK11 | NM_000455 |
| STK16 | NM_001008910 |
| STOM | NM_004099 |
| STOML1 | NM_004809 |
| STOML2 | NM_013442 |
| STRAP | NM_007178 |
| STRIP1 | NM_001270768 |
| STRN3 | NM_014574 |
| STT3A | NM_152713 |
| STT3B | NM_178862 |
| STUB1 | NM_005861 |
| STX10 | NM_003765 |
| STX17 | NM_017919 |
| STX4 | NM_004604 |
| STX3 | NM_003164 |
| STX8 | NM_004853 |
| STXBP3 | NM_007269 |
| STYXL1 | NM_016086 |
| SUB1 | NM_006713 |
| SUCLA2 | NM_003850 |
| SUCLG1 | NM_003849 |
| SUCLG2 | NM_003848 |
| SUGP1 | NM_172231 |
| SUGT1 | NM_006704 |
| SUMO1 | NM_003352 |
| SUMO3 | NM_006936 |
| SUN2 | NM_015374 |
| SUPT4H1 | NM_003168 |
| SUPT5H | NM_003169 |
| SUPT6H | NM_003170 |
| SUPT7L | NM_014860 |
| SUPV3L1 | NM_003171 |
| SURF1 | NM_003172 |
| SURF4 | NM_033161 |
| SURF6 | NM_006753 |
| SUV420H1 | NM_016028 |
| SUZ12 | NM_015355 |
| SYAP1 | NM_032796 |
| SYF2 | NM_015484 |
| SYMPK | NM_004819 |
| SYNCRIP | NM_006372 |
| SYNJ2BP | NM_018373 |
| SYNJ2BP- | NM_001202547 |
| COX16 | |
| SYPL1 | NM_006754 |
| SYS1 | NM_033542 |
| SYVN1 | NM_032431 |
| SZRD1 | NM_001114600 |
| TAB1 | NM_006116 |
| TAB2 | NM_015093 |
| TACO1 | NM_016360 |
| TADA1 | NM_053053 |
| TADA3 | NM_006354 |
| TAF10 | NM_006284 |
| TAF11 | NM_005643 |
| TAF12 | NM_005644 |
| TAF13 | NM_005645 |
| TAF15 | NM_003487 |
| TAF1D | NM_024116 |
| TAF4 | NM_003185 |
| TAF5L | NM_014409 |
| TAF8 | NM_138572 |
| TAF9 | NM_003187 |
| TALDO1 | NM_006755 |
| TAMM41 | NM_138807 |
| TANGO2 | NM_152906 |
| TANGO6 | NM_024562 |
| TANK | NM_004180 |
| TAOK2 | NM_004783 |
| TAPBP | NM_003190 |
| TAPT1 | NM_153365 |
| TARDBP | NM_007375 |
| TARS | NM_152295 |
| TATDN1 | NM_001146160 |
| TATDN2 | NM_014760 |
| TAX1BP1 | NM_006024 |
| TAZ | NM_000116 |
| TBC1D1 | NM_015173 |
| TBC1D14 | NM_001113363 |
| TBC1D15 | NM_022771 |
| TBC1D20 | NM_144628 |
| TBC1D22A | NM_014346 |
| TBC1D23 | NM_018309 |
| TBC1D7 | NM_016495 |
| TBC1D9B | NM_015043 |
| TBCA | NM_004607 |
| TBCB | NM_001281 |
| TBCC | NM_003192 |
| TBCCD1 | NM_018138 |
| TBCD | NM_005993 |
| TBCE | NM_003193 |
| TBK1 | NM_013254 |
| TBRG1 | NM_032811 |
| TBRG4 | NM_004749 |
| TCATM | NM_173826 |
| TCEANC2 | NM_153035 |
| TCEB1 | NM_005648 |
| TCEB2 | NM_007108 |
| TCEB3 | NM_003198 |
| TCERG1 | NM_006706 |
| TCF12 | NM_207040 |
| TCF20 | NM_005650 |
| TCF25 | NM_014972 |
| TCP1 | NM_030752 |
| TCTN3 | NM_015631 |
| TDP2 | NM_016614 |
| TDRD3 | NM_030794 |
| TECR | NM_138501 |
| TEF | NM_003216 |
| TEFM | NM_024683 |
| TELO2 | NM_016111 |
| TERF2 | NM_005652 |
| TERF2IP | NM_018975 |
| TEX2 | NM_018469 |
| TEX261 | NM_144582 |
| TEX264 | NM_015926 |
| TFAM | NM_003201 |
| TFB1M | NM_016020 |
| TFB2M | NM_022366 |
| TFCP2 | NM_005653 |
| TFDP1 | NM_007111 |
| TFE3 | NM_006521 |
| TFG | NM_006070 |
| TFIP11 | NM_012143 |
| TFPT | NM_013342 |
| TGIF2- | NM_001199535 |
| C20orf24 | |
| TGOLN2 | NM_006464 |
| THADA | NM_001271643 |
| THAP3 | NM_001195752 |
| THAP4 | NM_015963 |
| THAP5 | NM_182529 |
| THAP7 | NM_030573 |
| THOC5 | NM_003678 |
| THOC7 | NM_025075 |
| THOP1 | NM_003249 |
| THRAP3 | NM_005119 |
| THTPA | NM_024328 |
| THUMPD3 | NM_015453 |
| THYN1 | NM_014174 |
| TIA1 | NM_022007 |
| TIAL1 | NM_003252 |
| TICAM1 | NM_182919 |
| TIGD5 | NM_032862 |
| TIGD6 | NM_030953 |
| TIMM10 | NM_012456 |
| TIMM10B | NM_012192 |
| TIMM13 | NM_012458 |
| TIMM17A | NM_006335 |
| TIMM17B | NM_005834 |
| TIMM21 | NM_014177 |
| TIMM22 | NM_013337 |
| TIMM44 | NM_006351 |
| TIMM50 | NM_001001563 |
| TIMM8B | NM_012459 |
| TIMM9 | NM_012460 |
| TIMMDC1 | NM_016589 |
| TINF2 | NM_012461 |
| TIPRL | NM_152902 |
| TJAP1 | NM_080604 |
| TLE1 | NM_080604 |
| TLKI | NM_005077 |
| TM2D1 | NM_032027 |
| TM2D2 | NM_031940 |
| TM2D3 | NM_025141 |
| TM9SF1 | NM_006405 |
| TM9SF2 | NM_004800 |
| TM9SF3 | NM_020123 |
| TM9SF4 | NM_014742 |
| TMBIM1 | NM_022152 |
| TMBIM4 | NM_016056 |
| TMBIM6 | NM_003217 |
| TMCC1 | NM_001017395 |
| TMCO1 | NM_019026 |
| TMCO3 | NM_017905 |
| TMED1 | NM_006858 |
| TMED10 | NM_006827 |
| TMED2 | NM_006815 |
| TMED4 | NM_182547 |
| TMED5 | NM_016040 |
| TMED7 | NM_181836 |
| TMED7-TICAM2 | NM_001164468 |
| TMED9 | NM_017510 |
| TMEM101 | NM_032376 |
| TMEM106B | NM_018374 |
| THEM106C | NM_024056 |
| TMEM115 | NM_007024 |
| THEM120A | NM_031925 |
| TMEM126A | NM_032273 |
| TMEM127 | NM_017849 |
| TMEM128 | NM_032927 |
| TMEM129 | NM_138385 |
| TMEM131 | NM_015348 |
| TMEM134 | NM_025124 |
| TMEM141 | NM_032928 |
| TMEM147 | NM_032635 |
| TMEM14B | NM_030969 |
| TMEM14C | NM_016462 |
| TMEM161A | NM_017814 |
| TMEM167B | NM_020141 |
| TMEM168 | NM_022484 |
| TMEM177 | NM_030577 |
| TMEM179B | NM_199337 |
| TMEM18 | NM_152834 |
| TMEM184C | NM_018241 |
| TMEM185B | NM_024121 |
| TMEM186 | NM_015421 |
| TMEM187 | NM_003492 |
| TMEM189 | NM_199129 |
| TMEM189- | NM_199203 |
| UBE2V1 | |
| TMEM19 | NM_018279 |
| TMEM192 | NM_001100389 |
| TMEM199 | NM_152464 |
| TMEM203 | NM_053045 |
| TMEM205 | NM_033408 |
| TMEM214 | NM_017727 |
| TMEM219 | NM_194280 |
| TMEM222 | NM_032125 |
| TMEM223 | NM_001080501 |
| TMEM230 | NM_014145 |
| TMEM242 | NM_018452 |
| TMEM248 | NM_017994 |
| TMEM251 | NM_015676 |
| TMEM256 | NM_152766 |
| TMEM258 | NM_014206 |
| TMEM259 | NM_033420 |
| TMEM30A | NM_018247 |
| TMEM33 | NM_018126 |
| TMEM39A | NM_018266 |
| TMEM41A | NM_080652 |
| TMEM41B | NM_015012 |
| TMEM42 | NM_144638 |
| TMEM5 | NM_014254 |
| TMEM50A | NM_014313 |
| TMEM50B | NM_006134 |
| TMEM55B | NM_144568 |
| TMEM57 | NM_018202 |
| TMEM59 | NM_004872 |
| TMEM60 | NM_032936 |
| TMEM62 | NM_024956 |
| TMEM63B | NM_018426 |
| TMEM64 | NM_001008495 |
| TMEM66 | NM_016127 |
| TMEM69 | NM_016486 |
| TMEM70 | NM_017866 |
| TMEM81 | NM_203376 |
| TMEM87A | NM_001110503 |
| TMEM9 | NM_016456 |
| TMEM9B | NM_020644 |
| TMF1 | NM_007114 |
| TMLHE | NM_018196 |
| TMPO | NM_001032284 |
| TMUB1 | NM_031434 |
| TMUB2 | NM_024107 |
| TMX1 | NM_030722 |
| TMX2 | NM_015959 |
| TMX4 | NM_021156 |
| TNFAIP1 | NM_021137 |
| TNFAIP8L2- | NM_001204848 |
| SCNM1 | |
| TNIP1 | NM_006058 |
| TNKS2 | NM_025235 |
| TNPO1 | NM_002270 |
| TNPO3 | NM_012470 |
| TNRC6A | NM_014494 |
| TOB1 | NM_005749 |
| TOLLIP | NM_019009 |
| TOMM20 | NM_014765 |
| TOMM22 | NM_020243 |
| TOMM40 | NM_006114 |
| TOMM5 | NM_001001790 |
| TOMM6 | NM_001334493 |
| TOMM7 | NM_019059 |
| TOMM70A | NM_014820 |
| TOP1 | NM_003286 |
| TOP2B | NM_001068 |
| TOPORS | NM_005802 |
| TOR1A | NM_000113 |
| TOR1A1P2 | NM_022347 |
| TOR1B | NM_014506 |
| TOR3A | NM_022371 |
| TOX4 | NM_014828 |
| TP53RK | NM_033550 |
| TPCN1 | NM_017901 |
| TPD52L2 | NM_003288 |
| TPGS1 | NM_033513 |
| TPI1 | NM_000365 |
| TPP2 | NM_003291 |
| TPRA1 | NM_001136053 |
| TPRG1L | NM_183752 |
| TPRKB | NM_016058 |
| TPRN | NM_001128228 |
| TPST2 | NM_003595 |
| TRA2A | NM_013293 |
| TRA2B | NM_004593 |
| TRAF6 | NM_004620 |
| TRAF7 | NM_032271 |
| TRAP1 | NM_001272049 |
| TRAPPC1 | NM_021210 |
| TRAPPC10 | NM_003274 |
| TRAPPC11 | NM_021942 |
| TRAPPC12 | NM_016030 |
| TRAPPC13 | NM_024941 |
| TRAPPC2L | NM_016209 |
| TRAPPC3 | NM_014408 |
| TRAPPC4 | NM_016146 |
| TRAPPC5 | NM_174894 |
| TRAPPC6B | NM_177452 |
| TRAPPC8 | NM_014939 |
| TRAPPC9 | NM_031466 |
| TRIAP1 | NM_016399 |
| TRIM26 | NM_003449 |
| TRIM27 | NM_006510 |
| TRIM28 | NM_005762 |
| TRIM3 | NM_006458 |
| TRIM39 | NM_021253 |
| TRIM39-RPP21 | NM_001199119 |
| TRIM41 | NM_033549 |
| TRIM44 | NM_017583 |
| TRIM56 | NM_030961 |
| TRIM65 | NM_173547 |
| TRIM8 | NM_030912 |
| TRIP12 | NM_004238 |
| TRIP4 | NM_016213 |
| TRMT1 | NM_017722 |
| TRMT10C | NM_017819 |
| TRMT112 | NM_016404 |
| TRMT12 | NM_017956 |
| TRMT1L | NM_030934 |
| TRMT2A | NM_022727 |
| TRNAU1AP | NM_017846 |
| TRNT1 | NM_182916 |
| TRPC4AP | NM_015638 |
| TRPT1 | NM_031472 |
| TRUB2 | NM_015679 |
| TSC2 | NM_000548 |
| TSEN15 | NM_052965 |
| TSEN34 | NM_024075 |
| TSFM | NM_005726 |
| TSG101 | NM_006292 |
| TSN | NM_004622 |
| TSNAX | NM_005999 |
| TSPAN17 | NM_012171 |
| TSPAN31 | NM_005981 |
| TSPYL1 | NM_003309 |
| TSR1 | NM_018128 |
| TSR2 | NM_058163 |
| TSR3 | NM_001001410 |
| TSSC4 | NM_005706 |
| TSTA3 | NM_003313 |
| TSTD2 | NM_139246 |
| TTC1 | NM_003314 |
| TTC17 | NM_018259 |
| TTC19 | NM_001271420 |
| TTC32 | NM_001008237 |
| TTC33 | NM_012382 |
| TTC37 | NM_014639 |
| TTC4 | NM_004623 |
| TTC7B | NM_001010854 |
| TTC9C | NM_173810 |
| TTI1 | NM_014657 |
| TTI2 | NM_025115 |
| TUBA1B | NM_006082 |
| TUBA1C | NM_032704 |
| TUBB | NM_178014 |
| TUBD1 | NM_001193612 |
| TUBGCP2 | NM_006659 |
| TUBGCP4 | NM_014444 |
| TUFM | NM_003321 |
| TUSC2 | NM_007275 |
| TUT1 | NM_022830 |
| TVP23B | NM_016078 |
| TXLNA | NM_175852 |
| TXLNG | NM_018360 |
| TXN2 | NM_012473 |
| TXNDC11 | NM_015914 |
| TXNDC12 | NM_015913 |
| TXNDC15 | NM_024715 |
| TXNDC17 | NM_032731 |
| TXNDC9 | NM_005783 |
| TXNL1 | NM_004786 |
| TXNL4A | NM_006701 |
| TXNL4B | NM_017853 |
| TXNRD1 | NM_003330 |
| TYK2 | NM_003331 |
| TYW1 | NM_018264 |
| U2AF1 | NM_006758 |
| U2AF1L4 | NM_144987 |
| U2AF2 | NM_007279 |
| UAP1 | NM_003115 |
| UBA1 | NM_003334 |
| UBA2 | NM_005499 |
| UBA3 | NM_003968 |
| UBA5 | NM_024818 |
| UBA52 | NM_003333 |
| UBAC2 | NM_177967 |
| UBALD1 | NM_145253 |
| UBAP1 | NM_016525 |
| UBAP2L | NM_014847 |
| UBB | NM_018955 |
| UBC | NM_021009 |
| UBE2A | NM_003336 |
| UBE2B | NM_003337 |
| UBE2D2 | NM_003339 |
| UBE2D3 | NM_003340 |
| UBE2D4 | NM_015983 |
| UBE2E1 | NM_003341 |
| UBE2E2 | NM_152653 |
| UBE2E3 | NM_006357 |
| UBE2F | NM_080678 |
| UBE2G2 | NM_003343 |
| UBE2H | NM_003344 |
| UBE2I | NM_003345 |
| UBE2J1 | NM_016021 |
| UBE2J2 | NM_058167 |
| UBE2K | NM_005339 |
| UBE2L3 | NM_003347 |
| UBE2M | NM_003969 |
| UBE2N | NM_003348 |
| UBE2NL | NM_001012989 |
| UBE2Q1 | NM_017582 |
| UBE2R2 | NM_017311 |
| UBE2V1 | NM_021988 |
| UBE2V2 | NM_003350 |
| UBE2W | NM_018299 |
| UBE2Z | NM_023079 |
| UBE3A | NM_000462 |
| UBE3B | NM_130466 |
| UBE3C | NM_014671 |
| UBE4A | NM_004788 |
| UBE4B | NM_006048 |
| UBFD1 | NM_019116 |
| UBIAD1 | NM_013319 |
| UBL3 | NM_007106 |
| UBL4A | NM_014235 |
| UBL5 | NM_024292 |
| UBL7 | NM_032907 |
| UBOX5 | NM_014948 |
| UBP1 | NM_014517 |
| UBQLN1 | NM_013438 |
| UBQLN2 | NM_013444 |
| UBQLN4 | NM_020131 |
| UBR2 | NM_015255 |
| UBR7 | NM_175748 |
| UBTD1 | NM_024954 |
| UBTF | NM_014233 |
| UBXN2A | NM_181713 |
| UBXN4 | NM_014607 |
| UBXN6 | NM_025241 |
| UCHL3 | NM_006002 |
| UCHL5 | NM_015984 |
| UCK1 | NM_031432 |
| UCK2 | NM_012474 |
| UCKL1 | NM_017859 |
| UEVLD | NM_001040697 |
| UFC1 | NM_016406 |
| UFD1L | NM_005659 |
| UFL1 | NM_015323 |
| UFSP2 | NM_018359 |
| UGP2 | NM_006759 |
| UHRF1BP1L | NM_015054 |
| ULK1 | NM_003565 |
| ULK3 | NM_001099436 |
| UNC50 | NM_014044 |
| UNG | NM_003362 |
| UPF1 | NM_002911 |
| UPF2 | NM_015542 |
| UPF3B | NM_023010 |
| UPRT | NM_145052 |
| UQCC | NM_018244 |
| UQCR10 | NM_013387 |
| UQCR11 | NM_006830 |
| UQCRB | NM_006294 |
| UQCRC1 | NM_003365 |
| UQCRC2 | NM_003366 |
| UQCRHL | NM_001089591 |
| UQCRQ | NM_014402 |
| URGCP | NM_001077664 |
| URI1 | NM_003796 |
| URM1 | NM_030914 |
| UROD | NM_000374 |
| UROS | NM_000375 |
| USB1 | NM_024598 |
| USE1 | NM_018467 |
| USF1 | NM_207005 |
| USF2 | NM_003367 |
| USP10 | NM_005153 |
| USP14 | NM_005151 |
| USP16 | NM_006447 |
| USP19 | NM_006677 |
| USP22 | NM_015276 |
| USP25 | NM_013396 |
| USP27X | NM_001145073 |
| USP33 | NM_015017 |
| USP38 | NM_032557 |
| USP39 | NM_006590 |
| USP4 | NM_003363 |
| USP47 | NM_017944 |
| USP5 | NM_003481 |
| USP7 | NM_003470 |
| USP8 | NM_005154 |
| USP9X | NM_001039590 |
| UTP11L | NM_016037 |
| UTP14A | NM_006649 |
| UTP14C | NM_021645 |
| UTP15 | NM_032175 |
| UTP23 | NM_032334 |
| UTP3 | NM_020368 |
| UTP6 | NM_018428 |
| UXS1 | NM_025076 |
| UXT | NM_004182 |
| VAC14 | NM_018052 |
| VAMP3 | NM_004781 |
| VAMP5 | NM_006634 |
| VAPA | NM_003574 |
| VAPB | NM_004738 |
| VARS2 | NM_020442 |
| VBP1 | NM_003372 |
| VCP | NM_007126 |
| VDAC3 | NM_005662 |
| VEZT | NM_017599 |
| VIMP | NM_018445 |
| VMA21 | NM_001017980 |
| VPS16 | NM_022575 |
| VPS18 | NM_020857 |
| VPS25 | NM_032353 |
| VPS26A | NM_004896 |
| VPS26B | NM_052875 |
| VPS28 | NM_016208 |
| VPS29 | NM_016226 |
| VPS33A | NM_022916 |
| VPS36 | NM_016075 |
| VPS37A | NM_152415 |
| VPS4A | NM_013245 |
| VPS51 | NM_013265 |
| VPS52 | NM_022553 |
| VPS53 | NM_018289 |
| VPS72 | NM_005997 |
| VRK2 | NM_006296 |
| VRK3 | NM_016440 |
| VTA1 | NM_016485 |
| VTI1A | NM_145206 |
| VTI1B | NM_006370 |
| WAC | NM_016628 |
| WAPAL | NM_015045 |
| WARS2 | NM_015836 |
| WBP11 | NM_016312 |
| WBP1L | NM_017787 |
| WBP2 | NM_012478 |
| WBP4 | NM_007187 |
| WBSCR22 | NM_017528 |
| WDR1 | NM_005112 |
| WDR12 | NM_018256 |
| WDR13 | NM_017883 |
| WDR18 | NM_024100 |
| WDR20 | NM_144574 |
| WDR24 | NM_032259 |
| WDR25 | NM_024515 |
| WDR26 | NM_025160 |
| WDR3 | NM_006784 |
| WDR33 | NM_018383 |
| WDR36 | NM_139281 |
| WDR41 | NM_018268 |
| WDR43 | NM_015131 |
| WDR44 | NM_019045 |
| WDR45 | NM_007075 |
| WDR45B | NM_019613 |
| WDR46 | NM_005452 |
| WDR55 | NM_017706 |
| WDR59 | NM_030581 |
| WDR5B | NM_019069 |
| WDR6 | NM_018031 |
| WDR61 | NM_025234 |
| WDR70 | NM_018034 |
| WDR73 | NM_032856 |
| WDR74 | NM_018093 |
| WDR75 | NM_032168 |
| WDR77 | NM_024102 |
| WDR81 | NM_152348 |
| WDR83OS | NM_016148 |
| WDR85 | NM_138778 |
| WDR89 | NM_080666 |
| WDTC1 | NM_015023 |
| WIBG | NM_032345 |
| WIPI2 | NM_015610 |
| WIZ | NM_021241 |
| WRAP53 | NM_018081 |
| WRB | NM_004627 |
| WRNIP1 | NM_020135 |
| WSB2 | NM_018639 |
| WTAP | NM_004906 |
| WTH3DI | NM_001077637 |
| WWP1 | NM_007013 |
| WWP2 | NM_007014 |
| XIAP | NM_001167 |
| XPA | NM_000380 |
| XPC | NM_001145769 |
| XPNPEP1 | NM_020383 |
| XPQ1 | NM_003400 |
| XPO7 | NM_015024 |
| XPOT | NM_007235 |
| XRCC5 | NM_021141 |
| XRCC6 | NM_001469 |
| XYLT2 | NM_022167 |
| YAF2 | NM_005748 |
| YARS | NM_003680 |
| YARS2 | NM_001040436 |
| YIF1A | NM_020470 |
| YIF1B | NM_033557 |
| YIPF1 | NM_018982 |
| YIPF3 | NM_015388 |
| YIPF4 | NM_032312 |
| YIPF5 | NM_030799 |
| YIPF6 | NM_173834 |
| YKT6 | NM_006555 |
| YME1L1 | NM_014263 |
| YPEL2 | NM_001005404 |
| YRDC | NM_024640 |
| YTHDC1 | NM_133370 |
| YTHDF1 | NM_017798 |
| YTHDF2 | NM_016258 |
| YTHDF3 | NM_152758 |
| YWHAB | NM_003404 |
| YWHAE | NM_006761 |
| YY1 | NM_003403 |
| YY1AP1 | NM_018253 |
| ZADH2 | NM_175907 |
| ZBED4 | NM_014838 |
| ZBED6 | NM_001174108 |
| ZBTB1 | NM_014950 |
| ZBTB10 | NM_023929 |
| ZBTB11 | NM_014415 |
| ZBTB14 | NM_003409 |
| ZBTB17 | NM_003443 |
| ZBTB18 | NM_006352 |
| ZBTB21 | NM_020727 |
| ZBTB25 | NM_006977 |
| ZBTB33 | NM_006777 |
| ZBTB39 | NM_014830 |
| ZBTB44 | NM_014155 |
| ZBTB45 | NM_032792 |
| ZBTB5 | NM_014872 |
| ZBTB6 | NM_006626 |
| ZBTB7A | NM_015898 |
| ZBTB80S | NM_178547 |
| ZC3H10 | NM_032786 |
| ZC3H11A | NM_014827 |
| ZC3H13 | NM_015070 |
| ZC3H15 | NM_018471 |
| ZC3H18 | NM_144604 |
| ZC3H3 | NM_015117 |
| ZC3H7A | NM_014153 |
| ZC3H7B | NM_017590 |
| ZCCHC10 | NM_017665 |
| ZCCHC11 | NM_015269 |
| ZCCHC3 | NM_033089 |
| ZCCHC7 | NM_032226 |
| ZCCHC9 | NM_032280 |
| ZCRB1 | NM_033114 |
| ZDHHC14 | NM_024630 |
| ZDHHC16 | NM_198044 |
| ZDHHC2 | NM_016353 |
| ZDHHC3 | NM_016598 |
| ZDHHC4 | NM_018106 |
| ZDHHC5 | NM_015457 |
| ZDHHC8 | NM_013373 |
| ZFAND1 | NM_024699 |
| ZFAND2B | NM_138802 |
| ZFAND3 | NM_021943 |
| ZFAND5 | NM_006007 |
| ZFAND6 | NM_019006 |
| ZFP91 | NM_053023 |
| ZFPL1 | NM_006782 |
| ZFR | NM_016107 |
| ZFYVE1 | NM_021260 |
| AFYVE19 | NM_032850 |
| ZFYVE27 | NM_144588 |
| ZGPAT | NM_032527 |
| ZHX1 | NM_007222 |
| ZHX1-C8ORF76 | NM_001204180 |
| ZHX2 | NM_014943 |
| ZHX3 | NM_015035 |
| ZKSCAN1 | NM_003439 |
| ZMAT2 | NM_144723 |
| ZMAT3 | NM_022470 |
| ZMAT5 | NM_019103 |
| ZMPSTE24 | NM_005857 |
| ZMYM2 | NM_003453 |
| ZMYND11 | NM_006624 |
| ZNF121 | NM_001008727 |
| ZNF131 | NM_003432 |
| ZNF134 | NM_003435 |
| ZNF138 | NM_001271649 |
| ZNF142 | NM_001005537 |
| ZNF143 | NM_003442 |
| ZNF146 | NM_007145 |
| ZNF174 | NM_003450 |
| ZNF181 | NM_001029997 |
| ZNF189 | NM_197977 |
| ZNF195 | NM_007152 |
| ZNF197 | NM_006991 |
| ZNF207 | NM_003457 |
| ZNF22 | NM_006963 |
| ZNF226 | NM_015919 |
| ZNF232 | NM_014519 |
| ZNF24 | NM_006965 |
| ZNF259 | NM_003904 |
| ZNF274 | NM_016324 |
| ZNF277 | NM_021994 |
| ZNF280D | NM_017661 |
| ZNF281 | NM_012482 |
| ZNF3 | NM_017715 |
| ZNF32 | NM_006973 |
| ZNF322 | NM_024639 |
| ZNF326 | NM_182976 |
| ZNF330 | NM_014487 |
| ZNF335 | NM_022095 |
| ZNF33A | NM_006954 |
| ZNF343 | NM_024325 |
| ZNF347 | NM_032584 |
| ZNF37A | NM_003421 |
| ZNF384 | NM_133476 |
| ZNF394 | NM_032164 |
| ZNF397 | NM_001135178 |
| ZNF398 | NM_020781 |
| ZNF408 | NM_024741 |
| ZNF41 | NM_007130 |
| ZNF410 | NM_021188 |
| ZNF414 | NM_032370 |
| ZNF419 | NM_024691 |
| ZNF438 | NM_182755 |
| ZNF444 | NM_018337 |
| ZNF446 | NM_017908 |
| ZNF48 | NM_152652 |
| ZNF480 | NM_144684 |
| ZNF491 | NM_152356 |
| ZNF506 | NM_001099269 |
| ZNF507 | NM_014910 |
| ZNF513 | NM_001201459 |
| ZNF518A | NM_014803 |
| ZNF526 | NM_133444 |
| ZNF561 | NM_152289 |
| ZNF574 | NM_022752 |
| ZNF576 | NM_024327 |
| ZNF579 | NM_152600 |
| ZNF580 | NM_016202 |
| ZNF592 | NM_014630 |
| ZNF593 | NM_015871 |
| ZNF598 | NM_178167 |
| ZNF620 | NM_001056167 |
| ZNF622 | NM_033414 |
| ZNF623 | NM_014789 |
| ZNF638 | NM_014497 |
| ZNF639 | NM_016331 |
| ZNF641 | NM_152320 |
| ZNF644 | NM_016620 |
| ZNF649 | NM_023074 |
| ZNF654 | NM_018293 |
| ZNF655 | NM_138494 |
| ZNF664 | NM_152437 |
| ZNF668 | NM_024706 |
| ZNF672 | NM_024836 |
| ZNF687 | NM_020832 |
| ZNF688 | NM_145271 |
| ZNF691 | NM_015911 |
| ZNF7 | NM_003416 |
| ZNF706 | NM_016096 |
| ZNF721 | NM_133474 |
| ZNF740 | NM_001004304 |
| ZNF76 | NM_003427 |
| ZNF764 | NM_033410 |
| ZNF770 | NM_014106 |
| ZNF777 | NM_015694 |
| ZNF787 | NM_001002836 |
| ZNF805 | NM_001145078 |
| ZNF814 | NM_001144989 |
| ZNF830 | NM_052857 |
| ZNF865 | NM_001195605 |
| ZNF91 | NM_003430 |
| ZNHIT1 | NM_006349 |
| ZNHIT3 | NM_004773 |
| ZNRD1 | NM_014596 |
| ZRANB1 | NM_017580 |
| ZRANB2 | NM_005455 |
| ZSCAN21 | NM_145914 |
| ZSCAN29 | NM_152455 |
| ZSCAN32 | NM_017810 |
| ZSWIM1 | NM_080603 |
| ZSWIM7 | NM_001042697 |
| ZSWIM8 | NM_015037 |
| ZW10 | NM_004724 |
| ZXDA | NM_007156 |
| ZXDB | NM_007157 |
| ZZZ3 | NM_015534 |

TABLE 3

| | | | | | |
|---|---|---|---|---|---|
| ALYREF | DHX9 | KPNB1 | POLR21 | RPA2 | SNRPG |
| ARCN1 | DNM2 | LIAS | POMGNT2 | RPL10A | SNU13 |
| ATF7 | DYNLRB1 | LRRC59 | PPP2R1A | RPL11 | SON |
| ATP6V0D1 | EEF2 | LSM3 | PPP5C | RPL14 | SRSF1 |
| BCL2L1 | EFTUD2 | LSM4 | PRPF18 | RPL27 | SRSF3 |
| BRIX1 | EIF2B4 | LSM5 | PRPF19 | RPL30 | SUPT5H |
| C12orf66 | EIF2S2 | LSM6 | PRPF3 | RPL31 | SUPT6H |
| C14orf2 | EIF3A | MAPK6 | PRPF31 | RPL32 | SUPV3L1 |
| C16orf72 | EIF3B | MAPKAP1 | PRPF8 | RPL34 | TAF10 |
| CARS2 | EIF3D | MEN1 | PRUNE | RPL35 | TFCP2 |
| CCNL1 | EIF3E | MRPS31 | PSMA1 | RPL35A | TFIP11 |
| CCT3 | EIF3G | MSANTD3 | PSMA2 | RPL4 | THOP1 |
| CCT4 | EIF3I | NACA | PSMA3 | RPL6 | TIMM10 |
| CCT6A | EIF4A3 | NAPA | PSMA6 | RPN2 | TIMM50 |
| CCT7 | EIF5B | NDUFV2 | PSMB2 | RPS13 | TMEM5 |
| CCT8 | EIF6 | NEDD8 | PSMB3 | RPS24 | TSG101 |
| CDC40 | ELP4 | NUDT21 | PSMB1 | RPS5 | TSTA3 |
| CDC5L | ERH | NUP133 | PSMB7 | RPS6 | TUBA1B |
| CHD4 | ETF1 | NUP54 | PSMC2 | RRM1 | TUBA1C |
| CHMP2A | EX0SC10 | NXF1 | PSMC3 | RUVBL1 | TUBB |
| CLTC | FIP1LI | 0AZ2 | PSMC4 | SDAD1 | TUBGCP2 |
| CNOT3 | FLAD1 | PABPN1 | PSMD1 | SEC61A1 | U2AF1 |
| COPA | FTSJ3 | PAPOLA | PSMD11 | SF3A1 | U2AF2 |
| COPB1 | FUBP1 | PCBP2 | PSMD12 | SF3B1 | UBA1 |
| COFB2 | GNL3 | PELP1 | PSMD3 | SF3B2 | UBB |
| COPS2 | HAUS7 | PFDN2 | PSMD6 | SF3B3 | USP39 |
| COPS4 | EDAC3 | PHB | PSMD7 | SF3B4 | VCP |
| COPS6 | HNRNPC | PHB2 | PSMD8 | SF3B5 | VPS28 |
| COPS8 | HNRNPK | PHF5A | PUF60 | SKIV2L2 | WBP11 |
| COPZ1 | HNRNPL | PI4KA | QARS | SNAPC3 | WDR12 |
| DARS | HNRNPM | PLIN3 | RALY | SNRNP200 | WDR61 |
| DDB1 | HNRNPU | PLRG1 | RAN | SNRNP27 | WWP2 |
| DDX18 | HSPA9 | PNPT1 | RANBP2 | SNRNP70 | XP01 |
| DDX41 | IARS2 | POLR2A | RBM14 | SNRPC | YY1 |
| DDX46 | ICK | POLR2D | RBM17 | SNRPD1 | ZC3H13 |
| DDX49 | KARS | POLR2F | RBM39 | SNRPD2 | ZNF207 |
| ZNF22 | ZNF24 | | | | |

1-2. Selection of Cancer Cell Line and Target Mutation

Gene mutations common to the selected gene sets were extracted using WES/WGS public data of various cancer cell lines from cancer databases of Cancer Cell Line Encyclopedia (CCLE), National Cancer Institute (NCI), and Catalogue of Somatic Mutations in Cancer (COSMIC).

1-3. siRNA Synthesis

To inhibit protein expression of the cancer cell line mutant genes selected in Example 1-2, siRNAs were designed. Real time RT-PCR primers and probes for examining expression inhibitory efficiency of siRNAs were also designed such that they specifically react with only the mutant genes, and the results are shown in Table 4 below.

TABLE 4

| | | | Gene. | | | hg19.mut. | | | | |
|---|---|---|---|---|---|---|---|---|---|---|
| no | cell line | Tissue | name | refseq.id | chr | pos | aa | wild | mutant | siRNA |
| 1 | SNU-387 | liver | CNOT3 | NM_014516 | chr10 | 54656673 | p.D658D | C | T | CACTCGGATACTGTGGAAT |
| 2 | RD | soft_ | CNOT3 | NM_014516 | chr19 | 54649456 | p.K202K | G | A | GCAAAATCAAGGACGACGT |
| | | tissue | | | | | | | | |
| 3 | PANC-08-13 | pancreas | DDX46 | NM_014829 | chr5 | 134109517 | p.K193K | A | G | GAGATGAAGCAAGGGAAAA |
| 4 | NCI-H820 | lung | DDX49 | NM_019070 | chr19 | 19030598 | p.Q16Q | A | G | CGTGGAACAGTGTCGGCAG |
| 5 | Calu-3 | lung | HNRNPK | NM_031263 | chr9 | 86585178 | p.S420S | G | A | CGGGAGCTTCAATCAAAAT |
| 6 | NCI-H820 | | NXF1 | NM_006362 | chr11 | 62564024 | P.Y398Y | C | T | CAATTTATGACTCTGGAGA |
| 7 | NCI-H69 | lung | SNRNP200 | NM_014014 | chr2 | 96950285 | p.L1401L | C | A | GGACAGGCTAAACAAGAAG |
| 8 | NCI-H69 | | ZC3H13 | NM_015070 | chr13 | 46543189 | p.R1164R | C | A | CACAGAACAAGAGTAGAAA |
| 9 | MDA-MB-231 | breast | KARS | NM_005548 | chr16 | 75663394 | p.V490V | G | C | GGTGTTTGTGATGAAGAAA |
| 10 | SK_OV_3 | ovary: | EIF3D | NM_003753 | chr22 | 36912832 | p.K332K | C | T | GGGAAAGAAAGATACAACT |
| | | ascites | | | | | | | | |

1-4. Examination of Inhibition of Protein Expression of Cancer Cell Line Mutant Genes by siRNA

(1) RD Cell Line

To examine whether protein expression of target genes in cancer cell lines was inhibited by siRNAs designed in Example 1-3, Western blotting was performed. In detail, each 25 nM of Cont siRNA and CNOT3 siRNA designed in Example 3 were added to RD cells, respectively, followed by incubation for 24 hr. Thereafter, the incubated cells were harvested and proteins were extracted therefrom. Western blotting was performed to examine changes in CNOT3 gene expression.

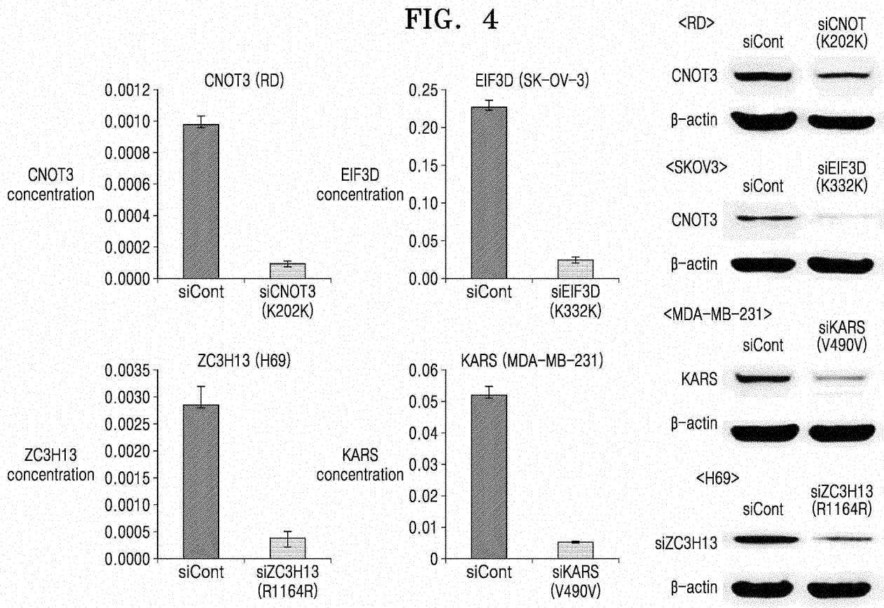

As a result, as shown in FIG. 4, the effect of CNOT3 siRNA on CNOT3 gene expression was confirmed in the soft tissue cell line treated with CNOT3 siRNA.

(2) NCI-H69 Cell Line

Experiments were performed in the same manner as in (1), except that NCI-H69 cell line and ZC3H13 siRNA were used.

As a result, as shown in FIG. 4, the inhibitory effect of ZC3H13 siRNA on ZC3H13 gene expression was confirmed in the lung cancer cell line treated with ZC3H13 siRNA.

(3) MDAMB-231 Cell Line

Experiments were performed in the same manner as in (1), except that MDAMB-231 cell line and KARS siRNA were used.

As a result, as shown in FIG. 4, the inhibitory effect of KARS siRNA on KARS gene expression was confirmed in the breast cancer cell line treated with KARS siRNA.

(4) SK-OV-3 Cell Line

Experiments were performed in the same manner as in (1), except that SK-OV-3 cell line and EIF3D siRNA were used.

As a result, as shown in FIG. 4, the inhibitory effect of EIF3D siRNA on SK-OV-3 gene expression was confirmed in the ovarian cancer cell line treated with EIF3D siRNA.

1-5. Examination of cytotoxicity of siRNA

(1) RD Cell Line

To examine effects of the siRNAs designed in Example 1-3 on cancer cell death, RD cells were treated with CNOT siRNA, and then changes in the increase rate of cytotoxicity were examined. In detail, RD cells were seeded at a density of 1×10⁵ cells/ml in a 6-well plate, and then each 25 nM of Cont siRNA and CNOT siRNA were added thereto, followed by incubation for 24 hr. Thereafter, annexin V staining was performed, and the increase rate of cytotoxicity was examined using a FACSCalibur flow cytometer (Becton Dickinson). Then, the increase rate of cytotoxicity was examined by measuring absorbance at 490 nm using a Pierce LDH Cytotoxicity Assay Kit (thermo scientific) which is a product to measure cell damage using lactate dehydrogenase (LDH) released from cells.

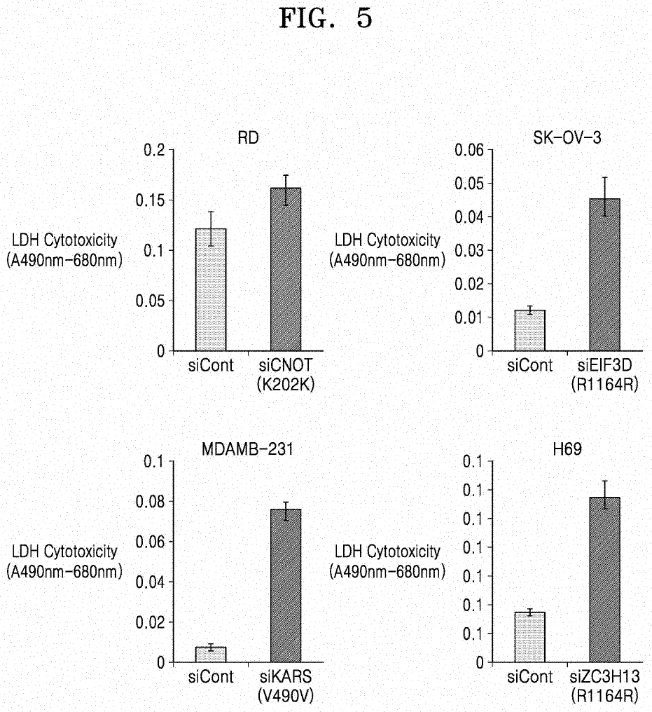

As a result, as shown in FIG. 5, it was confirmed that cytotoxicity was more increased in CNOT siRNA-treated cells.

(2) NCI-H69 Cell Line

Experiments were performed in the same manner as in (1), except that NCI-H69 cell line and ZC3H13 siRNA were used.

As a result, as shown in FIG. 5, it was confirmed that cytotoxicity was more increased in ZC3H13 siRNA-treated cells.

(3) MDAMB-231 Cell Line

Experiments were performed in the same manner as in (1), except that MDAMB-231 cell line and KARS siRNA were used.

As a result, as shown in FIG. 5, it was confirmed that cytotoxicity was more increased in KARS siRNA-treated cells.

(4) SK-OV-3 Cell Line

Experiments were performed in the same manner as in (1), except that SK-OV-3 cell line and EIF3D siRNA were used.

As a result, as shown in FIG. 5, it was confirmed that cytotoxicity was more increased in EIF3D siRNA-treated cells.

In other words, it was confirmed that cytotoxicity was increased in cells treated with siRNA against the target, indicating that the gene panel according to one specific embodiment may be usefully applied to a targeted therapy for an individual.

### Example 2. Personalized Therapy

2-1. Target Capture and Library Construction

For NGS experiment, genomic DNAs were isolated from cancer tissue specimens (tissue, blood, FFPE, FNA, etc.) derived from various cancer patients using a QiAmp DNA Mini kit (Qiagen, Valencia, Calif., USA). Thereafter, concentrations, purities, and degradation of the isolated genomic DNAs were determined using a Nanodrop 8000 UV-Vis spectrometer (Thermo Scientific Inc., DE, USA), a Qubit 2.0 Fluorometer (Life technologies Inc., Grand Island, N.Y., USA), and a 2200 TapeStation Instrument (Aglient Technologies, Santa Clara, Calif., USA). Specimens meeting the QC criteria were used in subsequent experiments.

The genomic DNA (˜250 ng) obtained from each tissue was subjected to shearing using Covaris S220 (Covaris, MA, USA), and then subjected to end-repair, A-tailing, paired-end adaptor ligation and amplification to construct sequencing libraries. To capture 220 genomic regions selected in Example 1, reaction was allowed using a composition containing all the prepared polynucleotides for a hybridization time of 24 hr at 65° C., and the genomic DNA library fragments captured by the hybridization were purified. Binding property of biotin and streptavidin attached to polynucleotides was used in the purification. In detail, after binding the streptavidin coated with magnetic beads and biotin attached to the captured library fragments, the captured library fragments were separated from the mixture using a magnetic force. Thereafter, the purified gene DNA library fragments were amplified in a PCR equipment with an index barcode tag, and the conditions are shown in Table 5 below.

TABLE 5

| Stage | Temperature | Time |
|---|---|---|
| 1 | 98° C. | 45 sec |
| 2 | 98° C. | 15 sec |
| 3 | 60° C. | 30 sec |
| 4 | 72° C. | 30 sec |

Repeating 2~3 stages for a total of 13 times.

| | | |
|---|---|---|
| 6 | 72° C. | 5 min |
| 7 | 4° C. | Storage |

2-2. Sequencing

The gene fragments captured in Example 2-1 were injected into an NGS instrument (Miseq, illumina, USA), and sequence information of each DNA fragment was obtained and aligned, thereby obtaining sequence information about each gene in the cancer sample. Sequencing reaction was performed using a TruSeq Rapid PE Cluster kit and a TruSeq Rapid SBS kit (Illumina, USA) under 100 bp paired-end conditions.

2-3. Variant Calling

Data of sequencing reads obtained in Example 2-2 were aligned to UCSC hg19 reference genome (http://genome.ucsc.edu) using Burrows-Wheeler Aligner (BWA) algorithm. PCR duplication was removed using a Picard-tools-1.8 (http://picard.sourceforge.net/), and single nucleotide variation (SNV) was identified using GATK-2.2.9 algorithm.

The above descriptions of the present disclosure are for illustrative purposes only. It will be understood by those skilled in the art that the present disclosure may be implemented in a different specific form without changing the technical spirit or essential characteristics thereof. Therefore, it should be understood that the above embodiments are not limitative, but illustrative in all aspects.

1. A gene panel for personalized medicine, the gene panel comprising 10 or more consecutive polynucleotides comprising mutations of polynucleotides of genes listed in Table 3 below, or complementary polynucleotides thereof:
TABLE 3

| | | | | | |
|---|---|---|---|---|---|
| ALYREF | DHX9 | KPNB1 | POLR2I | RPA2 | SNRPG |
| ARCN1 | DNM2 | LIAS | POMGNT2 | RPL10A | SNU13 |
| ATF7 | DYNLRB1 | LRRC59 | PPP2R1A | RPL11 | SON |
| ATP6V0D1 | EEF2 | LSM3 | PPP5C | RPL14 | SRSF1 |
| BCL2L1 | EFTUD2 | LSM4 | PRPF18 | RPL27 | SRSF3 |
| BRIX1 | EIF2B4 | LSM5 | PRPF19 | RPL30 | SUPT5H |
| C12orf66 | EIF2S2 | LSM6 | PRPF3 | RPL31 | SUPT6H |
| C14orf2 | EIF3A | MAPK6 | PRPF31 | RPL32 | SUPV3L1 |
| C16orf72 | EIF3B | MAPKAP1 | PRPF8 | RPL34 | TAF10 |
| CARS2 | EIF3D | MEN1 | PRUNE | RPL35 | TFCP2 |
| CCNL1 | EIF3E | MRPS31 | PSMA1 | RPL35A | TFIP11 |
| CCT3 | EIF3G | MSANTD3 | PSMA2 | RPL4 | THOP1 |
| CCT4 | EIF3I | NACA | PSMA3 | RPL6 | TIMM10 |
| CCT6A | EIF4A3 | NAPA | PSMA6 | RPN2 | TIMM50 |
| CCT7 | EIF5B | NDUFV2 | PSMB2 | RPS13 | TMEM5 |
| CCT8 | EIF6 | NEDD8 | PSMB3 | RPS24 | TSG101 |
| CDC40 | ELP4 | NUDT21 | PSMB4 | RPS5 | TSTA3 |
| CDC5L | ERH | NUP133 | PSMB7 | RPS6 | TUBA1B |
| CHD4 | ETF1 | NUP54 | PSMC2 | RRM1 | TUBA1C |
| CHMP2A | EXOSC10 | NXF1 | PSMC3 | RUVBL1 | TUBB |
| CLTC | FIPIL1 | 0AZ2 | PSMC4 | SDAD1 | TUBGCP2 |
| CNOT3 | FLAD1 | PABPN1 | PSMD1 | SEC61A1 | U2AF1 |
| COPA | FTS13 | PAPOLA | PSMD11 | SF3A1 | U2AF2 |
| COPB1 | FUBP1 | PCBP2 | PSMD12 | SF3B1 | UBA1 |
| COPB2 | GNL3 | PELP1 | PSMD3 | SF3B2 | UBB |
| COPS2 | HAUS7 | PFDN2 | PSMD6 | SF3B3 | USP39 |
| COPS4 | HDAC3 | PHB | PSMD7 | SF3B4 | VCP |
| COPS6 | HNRNPC | PHB2 | PSMD8 | SF3B5 | VPS28 |
| COPS8 | HNRNPK | PHP5A | PUF60 | SKIV2L2 | WBP11 |
| COPZ1 | HNRNPL | PI4KA | QARS | SNAPC3 | WDR12 |
| DARS | HNRNPM | PLIN3 | RALY | SNRNP200 | WDR61 |
| DDB1 | HNRNPU | PLRG1 | RAN | SNRNP27 | WWP2 |
| DDX18 | HSPA9 | PNPT1 | RANBP2 | SNRNP70 | XPO1 |
| DDX41 | IARS2 | POLR2A | RBM14 | SNRPC | YY1 |
| DDX46 | ICK | POLR2D | RBM17 | SNRPD1 | ZC3H13 |
| DDX49 | KARS | POLR2F | RBM39 | SNRPD2 | ZNF207 |
| ZNF22 | ZNF24 | | | | |

2. The gene panel for personalized medicine of claim 1, wherein the polynucleotides are primers, probes, or antisense nucleic acids.

3. The gene panel for personalized medicine of claim 1, wherein the polynucleotides are labeled with a detectable label.

4. The gene panel for personalized medicine of claim 1, wherein the mutations are silent mutations.

5. The gene panel for personalized medicine of claim 1, wherein the mutations of the polynucleotides are T at position of hg19. 54656673 in SEQ ID NO: 1, A at position of hg19. 54649456 in SEQ ID NO: 2, G at position of hg19. 134109517 in SEQ ID NO: 3, G at position of hg19. 19030598 in SEQ ID NO: 4, A at position of hg19. 86585178 in SEQ ID NO: 5, T at position of hg19. 62564024 in SEQ ID NO: 6, A at position of hg19. 96950285 in SEQ ID NO: 7, A at position of hg19. 46543189 in SEQ ID NO: 8, C at position of hg19. 75663394 in SEQ ID NO: 9, and T at position of hg19. 36912832 in SEQ ID NO: 10.

6. The gene panel for personalized medicine of claim 1, wherein the gene panel is for targeted treatment of cancer or an intractable disease.

7. The gene panel for personalized medicine of claim 6, wherein the cancer is selected from the group consisting of brain cancer, gastric cancer, lung cancer, breast cancer, ovarian cancer, liver cancer, bronchial cancer, nasopharyngeal cancer, laryngeal cancer, esophageal cancer, pancreatic cancer, bladder cancer, prostate cancer, colorectal cancer, colon cancer, bone cancer, skin cancer, thyroid cancer, parathyroid cancer, ureter cancer, and cervical cancer.

8. The gene panel for personalized medicine of claim 6, wherein the intractable disease is selected from the group consisting of albinism, alcaptonuria, lactose intolerance, hereditary hemorrhagic telangiectasia, thalassemia, congenital dyserythropoietic anaemia, Evans syndrome, pituitary hypofunction, Huntington's disease, hereditary motor sensory neuropathy, and other autonomic nervous system disorders.

9. A method of constructing a gene panel for personalized medicine, the method comprising constructing a target gene set by screening the intersection of essential genes and constitutive genes.

10. The method of constructing a gene panel for personalized medicine of claim 9, the method comprising extracting mutant genes common to the target gene set from a plurality of public cell line databases, and collecting base sequence information of the mutant genes; and
verifying each mutant gene from the base sequence information of the mutant genes.

11. A method of providing information for personalized treatment, the method comprising
detecting mutations of the gene panel defined in claim 1 in a biological sample isolated from an individual; and
from the detection results, determining, as a treatment target for the individual, genes in which mutations have occurred.

12. The method of providing information for personalized treatment of claim 11, wherein when the mutations of the gene panel are T at position of hg19. 54656673 in SEQ ID NO: 1, A at position of hg19. 54649456 in SEQ ID NO: 2, G at position of hg19. 134109517 in SEQ ID NO: 3, G at position of hg19. 19030598 in SEQ ID NO: 4, A at position of hg19. 86585178 in SEQ ID NO: 5, T at position of hg19. 62564024 in SEQ ID NO: 6, A at position of hg19. 96950285 in SEQ ID NO: 7, A at position of hg19. 46543189 in SEQ ID NO: 8, Cat position of hg19. 75663394 in SEQ ID NO: 9, and T at position of hg19. 36912832 in SEQ ID NO: 10, it is determined that the individual belongs to a group with a high risk of developing cancer caused by gene mutations.
